(19) United States
(12) Patent Application Publication
(10) Pub. No.: US 2009/0326029 A1
(43) Pub. Date: Dec. 31, 2009

(54) NON-CYCLIC SUBSTITUTED BENZIMIDAZOLE THIOPHENE BENZYL ETHER COMPOUNDS

(76) Inventors: Kevin Wayne Kuntz, Durham, NC (US); Holly Kathleen Emerson, Durham, NC (US); Mui Cheung, Durham, NC (US); Jennifer Gabriel Badiang, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/303,119

(22) PCT Filed: May 31, 2007
(86) PCT No.: PCT/US2007/070108

§ 371 Date: Dec. 2, 2008

Related U.S. Application Data
(60) Provisional application No. 60/810,315, filed on Jun. 2, 2006.

(51) Int. Cl. A61K 31/4184; C07D 235/04; A61P 35/00

(52) U.S. Cl. 514/394; 548/304.7; 548/306.1

(57) ABSTRACT

The present invention provides benzimidazole thiophene compounds pharmaceutical compositions containing the same, processes for preparing the same and their use as pharmaceutical agents.

### BACKGROUND OF THE INVENTION

The present invention relates to novel benzimidazole thiophene compounds, pharmaceutical formulations comprising these compounds, and the use of these compounds in therapy.

Polo-like kinases (“PLK”) are evolutionarily conserved serine/threonine kinases that play critical roles in regulating processes in the cell cycle. PLK plays a role in the entry into and the exit from mitosis in diverse organisms from yeast to mammalian cells. PLK includes PLK1, PLK2, PLK3 and PLK4.

Overexpression of PLK1 appears to be strongly associated with neoplastic cells (including cancers). A published study has shown high levels of PLK1 RNA expression in >80% of lung and breast tumors, with little to no expression in adjacent normal tissue. Several studies have shown correlations between PLK expression, histological grade, and prognosis in several types of cancer. Significant correlations were found between percentages of PLK-positive cells and histological grade of ovarian and endometrial cancer (P<0.001). These studies noted that PLK is strongly expressed in invading endometrial carcinoma cells and that this could reflect the degree of malignancy and proliferation in endometrial carcinoma. Using RT-PCR analysis, PLK overexpression was detected in 97% of esophageal carcinomas and 73% of gastric carcinomas as compared to the corresponding normal tissues. Further, patients with high levels of PLK overexpression in esophageal carcinoma represented a significantly poorer prognosis group than those with low levels of PLK overexpression. In head and neck cancers, elevated mRNA expression of PLK1 was observed in most tumors; a Kaplan-Meier analysis showed that those patients with moderate levels of PLK1 expression survived longer than those with high levels of PLK1 expression. Analysis of patients with non-small cell lung carcinoma showed similar outcomes related to PLK1 expression.

PCT Publication No. WO2004/014899 to SmithKline Beecham discloses novel benzimidazole thiophene compounds of formula (I):

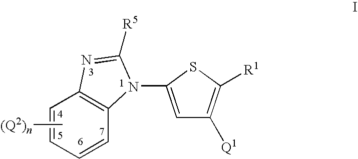

wherein:
R¹ is selected from the group consisting of H, alkyl, alkenyl, alkynyl, —C(O)R⁷, —CO₂R⁷, —C(O)NR⁷R⁸, —C(O)N(R⁷)OR⁸, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷, —S(O)₂NR⁷R⁸, —S(O)₂N(R⁷)-Ph, —S(O)₂N(R⁷)—R²-Ph, —NR⁷R⁸, N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula: —(R²)ₐ—(Y¹)_{b}—(R²)_{c}—R³
a, b and c are the same or different and are each independently 0 or 1 and at least one of a or b is 1;
n is 0, 1, 2, 3 or 4;
Q² is a group of formula: —(R²)ₐₐ—(Y²)_{bb}—(R²)_{cc}—R⁴ or two adjacent Q² groups are selected from the group consisting of alkyl, alkenyl, —OR^{X}, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula

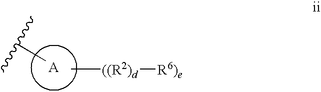

wherein:
Ring A is selected from the group consisting of C₅₋₁₀cycloalkyl, C₅₋₁₀cycyloalkenyl, aryl, 5-10 membered heterocycle having 1, 2 or 3 heteroatoms selected from N, O and 5 and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷, —CO₂—R₂-Ph, —CO₂—R²—Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, —C(S)NR⁷R⁸, —C(═NR⁷)R³, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²—Het, —N(R⁷)S(O)₂R⁸, —N(R⁷)—R²—S(O)₂R⁸, —NO₂, —CN and —N₃;
wherein when Q¹ is defined where b is 1 and c is 0, R³ is not halo, —C(O)R⁷, —C(O)NR⁷R⁸, —CO₂R⁷, —C(S)R⁷, —C(S)NR⁷R⁸, —C(═NR⁷)R³, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
wherein when Q² is defined where bb is 1 and cc is 0, R⁴ is not halo, —C(O)R⁷, —C(O)NR⁷R⁸, —CO₂R⁷, —C(S)R⁷, —C(S)NR⁷R⁸, —C(═NR⁷)R³, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R³ are the same or different and are each independently selected from the group consisting of H, alkyl, alkenyl, alkynyl, cycloalkyl and cycloalkenyl;
wherein when R¹ is —CO₂CH₃ and n is 0, Q¹ is not —OH;
   or a pharmaceutically acceptable salt, solvate or physiologically functional derivative thereof.

Also disclosed are pharmaceutical compositions containing these compounds, processes for their preparation and methods for treatment of conditions mediated by PLK using these compounds.

### BRIEF SUMMARY OF THE INVENTION

According to a first aspect of the invention there is provided compounds of formula (I):

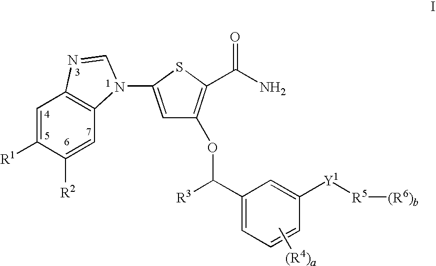

wherein:
R¹ and R² are the same or different and are each selected from H, halo, alkyl, haloalkyl, —OR⁷, —O-haloalkyl, —CN, —S(O)₂R⁷, —R⁵—S(O)₂R⁷, —NR⁷R⁸, and Het¹;
   Het¹ is a 5-6 membered heteroaryl having 1 or 2 heteroatoms selected from N, O and S, optionally substituted 1 or 2 times with a substituent selected from alkyl and oxo;
R³ is H or alkyl;
a is 0, 1 or 2;
each R⁴ is the same or different and is halo;
Y¹ is —O—, —N(R⁷)—, —C(O)N(H)— or —N(H)C(O)—;
R⁵ is C₁₋₃alkylene;
b is 1 or 2;
each R⁶ is the same or different and is independently selected from —OR⁷ and —NR⁷R⁸; and
each R⁷ and each R⁸ are the same or different and are each independently selected from H, alkyl, alkenyl, alkynyl, cycloalkyl and cycloalkenyl;
   and pharmaceutically acceptable salts and solvates thereof.

In one particular aspect, the present invention provides an enantiomerically enriched compound according to claim 1, having the stereochemistry depicted in formula (I-1):

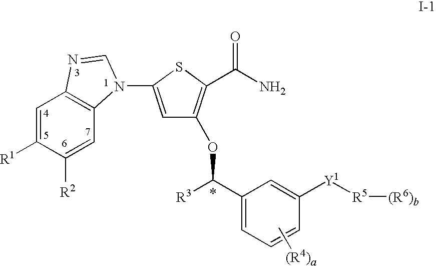

wherein * indicates the chiral carbon and all variables are as defined in claim 1.

In a third aspect, the present invention provides a pharmaceutical composition comprising a compound of formula (I) or (I-1). The composition may further comprise a pharmaceutically acceptable carrier, diluent or excipient.

In a fourth aspect, the present invention provides a method for treating a susceptible neoplasm in a mammal in need thereof. The method comprises administering to the mammal a therapeutically effective amount of a compound of formula (I) or (I-1). The susceptible neoplasm may be selected from the group consisting of breast cancer, colon cancer, small cell lung cancer, non-small cell lung cancer, prostate cancer, endometrial cancer, gastric cancer, melanoma, ovarian cancer, pancreatic cancer, squamous cell carcinoma, carcinoma of the head and neck, esophageal carcinoma, hepatocellular carcinoma, and hematologic malignancies.

In a fifth aspect, the present invention provides a method for treating a condition characterized by inappropriate cellular proliferation in a mammal in need thereof. The method comprising administering to the mammal a therapeutically effective amount of a compound of formula (I) or (I-1).

In a sixth aspect the present invention provides a process for preparing a compound of formula (I) or (I-1) wherein Y¹ is —O—. The process comprises the steps of:
a) reacting the compound of formula (VII):

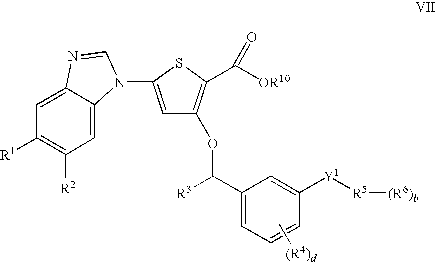

wherein R¹⁰ is selected from alkyl and suitable carboxylic acid protecting groups, and all other variables are as defined above,
   with ammonia to prepare a compound of formula (I);
b) optionally separating the compound of formula (I) into enantiomers;
c) optionally converting the compound of formula (I) to a pharmaceutically acceptable salt or solvate thereof; and
d) optionally converting the compound of formula (I) or a pharmaceutically acceptable salt or solvate thereof to a different compound of formula (I) or a pharmaceutically acceptable salt or solvate thereof.

In a seventh aspect the present invention provides a process for preparing a compound of formula (I) or (I-1) wherein Y¹ is —N(R⁷)— or —NHC(O)—. The process comprises the steps of:
a) reacting the compound of formula (XXXIII):

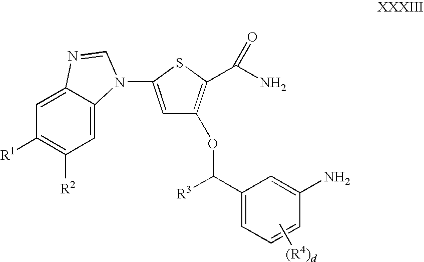

wherein all other variables are as defined above,
b) with a compound of formula (XXXIV) or (XXXV):

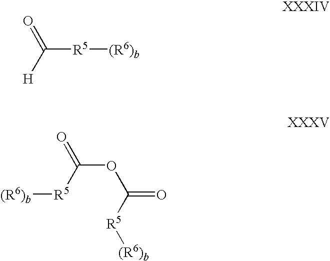

to prepare a compound of formula (I);
c) optionally separating the compound of formula (I) into enantiomers;
d) optionally converting the compound of formula (I) to a pharmaceutically acceptable salt or solvate thereof; and
e) optionally converting the compound of formula (I) or a pharmaceutically acceptable salt or solvate thereof to a different compound of formula (I) or a pharmaceutically acceptable salt or solvate thereof.

In another aspect, the present invention provides a compound of formula (I) or (I-1) or a pharmaceutically acceptable salt or solvate thereof for use in therapy.

In yet another aspect, the present invention provides a compound of formula (I) or (I-1) or a pharmaceutically acceptable salt or solvate thereof for use in the treatment of a condition mediated by PLK in a mammal in need thereof.

In yet another aspect, the present invention provides a compound of formula (I) or (I-1) or a pharmaceutically acceptable salt or solvate thereof for use in the treatment of a susceptible neoplasm, such as breast cancer, colon cancer, small cell lung cancer, non-small cell lung cancer, prostate cancer, endometrial cancer, gastric cancer, melanoma, ovarian cancer, pancreatic cancer, squamous cell carcinoma, carcinoma of the head and neck, esophageal carcinoma, hepatocellular carcinoma, and hematologic malignancies in a mammal.

In another aspect, the present invention provides a compound of formula (I) or (I-1) or a pharmaceutically acceptable salt or solvate thereof, for use in the treatment of a condition characterized by inappropriate cellular proliferation.

In yet another aspect, the present invention provides the use of a compound of formula (I) or (I-1) or a pharmaceutically acceptable salt or solvate thereof, for the preparation of a medicament for the treatment of condition mediated by PLK in a mammal.

In yet another aspect, the present invention provides the use of a compound of formula (I) or (I-1) or a pharmaceutically acceptable salt or solvate thereof, for the preparation of a medicament for the treatment of a susceptible neoplasm (e.g., breast cancer, colon cancer, small cell lung cancer, non-small cell lung cancer, prostate cancer, endometrial cancer, gastric cancer, melanoma, ovarian cancer, pancreatic cancer, squamous cell carcinoma, carcinoma of the head and neck, esophageal carcinoma, hepatocellular carcinoma, and hematologic malignancies) in a mammal.

In yet another aspect, the present invention provides the use of a compound of formula (I) or (I-1) or a pharmaceutically acceptable salt or solvate thereof, for the treatment of a condition characterized by inappropriate cellular proliferation in a mammal.

In yet another aspect, the present invention provides a pharmaceutical composition comprising a compound of formula (I) or (I-1) or a pharmaceutically acceptable salt or solvate thereof, for use in the treatment of a susceptible neoplasm, such as breast cancer, colon cancer, small cell lung cancer, non-small cell lung cancer, prostate cancer, endometrial cancer, gastric cancer, melanoma, ovarian cancer, pancreatic cancer, squamous cell carcinoma, carcinoma of the head and neck, esophageal carcinoma, hepatocellular carcinoma, and hematologic malignancies, in a mammal.

### DETAILED DESCRIPTION OF THE INVENTION

As used herein, “compound(s) of the invention” means a compound having a structural formula within the definition of formula (I) or (I-1) or a pharmaceutically acceptable salt or solvate thereof. Also, with respect to isolatable intermediates such as for example, compounds of formula (V) and (VII) (among others described below) the phrase “a compound of formula (number)” means a compound having that formula and pharmaceutically acceptable salts and solvates thereof.

As used herein, the terms “alkyl” (and “alkylene”) refer to straight or branched hydrocarbon chains containing from 1 to 8 carbon atoms. Examples of “alkyl” as used herein include, but are not limited to, methyl, ethyl, n-propyl, isopropyl, n-butyl, isobutyl, tert-butyl and n-pentyl. Examples of “alkylene” as used herein include, but are not limited to, methylene, ethylene, propylene, isopropylene, butylene, and isobutylene.

The term “haloalkyl” refers to alkyl (as defined above) substituted one or more times with a halogen. Thus, the term “haloalkyl” includes perhaloalkyls such as trifluoromethyl, as well as trifluoroethyl, among other halogenated alkyls.

As used herein, the term “alkenyl” (and “alkenylene”) refers to straight or branched hydrocarbon chains containing from 2 to 8 carbon atoms (unless a different number of atoms is specified) and at least one and up to three carbon-carbon double bonds. Examples of “alkenyl” as used herein include, but are not limited to ethenyl and propenyl. Examples of “alkenylene” as used herein include, but are not limited to ethenylene and propenylene.

As used herein, the term “alkynyl” refers to straight or branched hydrocarbon chains containing from 2 to 8 carbon atoms (unless a different number of atoms is specified) and at least one and up to three carbon-carbon triple bonds. Examples of “alkynyl” as used herein include, but are not limited to ethynyl and propynyl.

As used herein, the term “cycloalkyl” refers to a non-aromatic monocyclic carbocyclic ring having from 3 to 8 carbon atoms (unless a different number of atoms is specified) and no carbon-carbon double bonds. “Cycloalkyl” includes by way of example cyclopropyl, cyclobutyl, cyclopentyl, cyclohexyl, cycloheptyl and cyclooctyl. “Cycloalkyl” also includes substituted cycloalkyl. The cycloalkyl may optionally be substituted on any available carbon with one or more substituents selected from the group consisting of halo, C₁₋₃alkyl and C₁₋₃haloalkyl. Preferred cycloalkyl groups include C₃₋₆cycloalkyl and substituted C₃₋₆cycloalkyl.

As used herein, the term “cycloalkenyl” refers to a non-aromatic monocyclic carbocyclic ring having from 3 to 8 carbon atoms (unless a different number of atoms is specified) and up to 3 carbon-carbon double bonds. “Cycloalkenyl” includes by way of example cyclobutenyl, cyclopentenyl and cyclohexenyl. “Cycloalkenyl” also includes substituted cycloalkenyl. The cycloalkenyl may optionally be substituted on any available carbon with one or more substituents selected from the group consisting of halo, C₁₋₃alkyl and C₁₋₃haloalkyl.

The term “halo” or “halogen” refers to fluorine, chlorine, bromine and iodine.

The term “oxo” as used herein refers to the group ═O attached directly to a carbon atom of a hydrocarbon ring (i.e., cycloalkenyl, aryl, heterocycle or heteroaryl ring) as well as —N-oxides, sulfones and sulfoxides wherein the N or S are atoms of a heterocyclic or heteroaryl ring.

The term “heteroaryl” refers to aromatic monocyclic groups and fused bicyclic groups wherein at least one ring is aromatic, having the specified number of members and containing 1, 2, 3, or 4 heteroatoms selected from N, O and S (unless a different number of heteroatoms is specified). Examples of particular heteroaryl groups include but are not limited to furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole.

The term “members” (and variants thereof e.g., “membered”) in the context of heteroaryl groups refers to the total atoms, carbon and heteroatoms N, O and/or S, which form the ring. Thus, an example of a 6-membered heteroaryl ring is pyridine.

As used herein, the term “optionally” means that the subsequently described event(s) may or may not occur, and includes both event(s) that occur and events that do not occur.

The present invention provides compounds of formula (I):

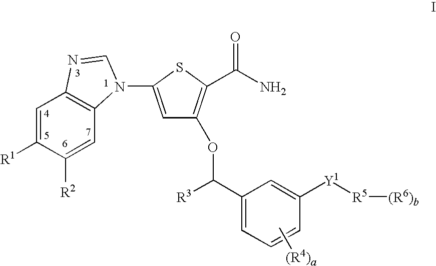

wherein:
R¹ and R² are the same or different and are each selected from H, halo, alkyl, haloalkyl, —OR⁷, —O-haloalkyl, —CN, —S(O)₂R^{X}—R⁵—S(O)₂R⁷, —NR⁷R⁸, and Het¹;
   Het¹ is a 5-6 membered heteroaryl having 1 or 2 heteroatoms selected from N, O and S, optionally substituted 1 or 2 times with a substituent selected from alkyl and oxo;
R³ is H or alkyl;
a is 0, 1 or 2;
each R⁴ is the same or different and is halo;
Y¹ is —O—, —N(R⁷)—, —C(O)N(H)— or —N(H)C(O)—;
R⁵ is C₁₋₃alkylene;
b is 1 or 2;
each R⁶ is the same or different and is independently selected from —OR⁷ and —NR⁷R⁸; and
each R⁷ and each R⁸ are the same or different and are each independently selected from H, alkyl, alkenyl, alkynyl, cycloalkyl and cycloalkenyl;
   or a pharmaceutically acceptable salt or solvate thereof.

In one embodiment, the compounds of formula (I) are defined wherein R¹ is selected from H, halo, —OR⁷, and Het¹, or any subset thereof. In one particular embodiment, R¹ is halo. In one particular embodiment, R¹ is —OR⁷. In one particular embodiment, R¹ is Het¹. In a specific embodiment, R¹ is selected from H, Cl, —O-alkyl, pyrrole, pyrazole and imidazole, or any subset thereof. In another embodiment, R¹ is selected from H, Cl, —O-alkyl, and pyrazole, or any subset thereof. In one particular embodiment, R¹ is H. In one particular embodiment, R¹ is Cl. In one particular embodiment, R¹ is —O—C₁₋₃alkyl. In one particular embodiment, R¹ is pyrazole.

In one embodiment, the compounds of formula (I) are defined wherein R² is selected H, halo, and —OR⁷, or any subset thereof. In one particular embodiment, R² is —OR⁷. In one particular embodiment, R² is H. In one particular embodiment, R² is halo. In one particular embodiment, R² is —O—Cl₁₋₃alkyl.

In one embodiment of the present invention, the compounds of formula (I) are defined wherein both R¹ and R² are the same and are H. In another embodiment, both R¹ and R² are the same and are —O—C₁₋₃alkyl. In another embodiment, R¹ is Het¹ (e.g., pyrazole) and R² is H. In another embodiment, at least one of R¹ and R² is halo, such as chloro.

In one embodiment, the compounds of formula (I) are defined wherein Het¹ is a 5-membered heteroaryl having 1 or 2 heteroatoms selected from N, O and S, optionally substituted 1 or 2 times with a substituent selected from alkyl and oxo. In another embodiment, Het¹ is a 5-membered heteroaryl having 1 or 2 nitrogen atoms, optionally substituted 1 or 2 times with a substituent selected from C₁₋₃alkyl and oxo. In a further embodiment, Het¹ is selected from pyrrole, pyrazole and imidazole, each optionally substituted 1 or 2 times with a substituent selected from C₁₋₃alkyl and oxo. Specific examples of groups defining Het¹ include but are not limited to pyrazole, N-methylpyrazole and N-oxo pyrazole; pyrrole, N-methylpyrrole and N-oxo pyrrole; and imidazole or methyl imidazole.

In one embodiment, the compounds of formula (I) are defined wherein R³ is alkyl. In one embodiment, R³ is C₁₋₃alkyl. In one preferred embodiment, R³ is methyl.

In one embodiment, the compounds of formula (I) are defined wherein a is 0 or 1. In one particular embodiment, a is 1.

In one embodiment, the compounds of formula (I) are defined wherein a is 1 or 2 and each R⁴ is the same or different and is selected from Cl and F. In one particular embodiment, a is 1 and R⁴ is Cl.

In one embodiment, the compounds of formula (I) are defined wherein Y¹ is —O—, —N(R⁷)— or —C(O)N(H)—. In one embodiment, the compounds of formula (I) are defined wherein Y¹ is —O—.

In one particular embodiment, the compounds of formula (I) are defined wherein R⁵ is C₂₋₃alkylene. In one embodiment, R⁵ is ethylene or n-propylene.

In one embodiment, the compounds of formula (I) are defined wherein b is 1.

In one embodiment, the compounds of formula (I) are defined wherein R⁶ is the same or different and is independently selected from —OH, —O-alkyl, —NH₂, —N(H)alkyl, and —N(alkyl)₂, or any subset thereof. In one embodiment, each R⁶ is the same or different and is independently selected from —OH, —O—C₁₋₃alkyl, —NH₂, —N(H)C₁₋₃alkyl, and —N(C₁₋₃alkyl)₂, or any subset thereof. In one embodiment, each R⁶ is the same or different and is independently selected from —OH, —NH₂ and —N(CH₃)₂, or any subset thereof.

In one embodiment, the compounds of formula (I) are defined wherein each R⁷ and each R⁸ are the same or different and are each independently selected from H, alkyl and alkenyl, or any subset thereof. In one embodiment, each R⁷ and each R³ are the same or different and are each independently selected from H and alkyl. In one embodiment, each R⁷ and each R⁸ are the same or different and are each independently selected from H and C₁₋₃alkyl.

Compounds of the invention exist in stereoisomeric forms (e.g. they contain one or more chiral or asymmetric carbon atoms). The term “chiral” refers to a molecule that is not superimposable on its mirror image. The term “achiral” refers to a molecule that is superimposable on its mirror image.

The term “stereoisomers” refers to compounds which have a common chemical constitution but differ in the arrangement of the atoms or groups in space. Stereoisomers may be optical isomers or geometric isomers. Optical isomers include both enantiomers and diastereomers. An “enantiomer” is one of a pair of optical isomers containing a chiral carbon atom whose molecular configuration have left- and right-hand (chiral) forms. That is, “enantiomer” refers to each of a pair of optical isomers of a compound which are non-superimposable mirror images of one another. A “diastereomer” is one of a pair of optical isomers of a compound with two or more centers of dissymmetry and whose molecules are not mirror images of one another. The nomenclature of a chiral center is governed by the (R) —(S) system. Whether a particular compound is designated as the “R” or “S” enantiomer according to the system depends upon the nature of the atoms or groups which are bound to the chiral carbon.

Enantiomers differ in their behavior toward plane-polarized light, that is, their optical activity. An enantiomer that rotates plane-polarized light in a clockwise direction is said to be dextrorotatory and is designated by the symbol “d” or “(+)” for positive rotation. An enantiomer that rotates plane-polarized light in the counterclockwise direction is said to be levorotatory and is designated by the symbol “l” or “(−)” for negative rotation. There is no correlation between the configuration of enantiomers and the direction in which they rotate plane-polarized light. There is also no necessary correlation between the (R) and (S) designation and the direction of rotation of the plane-polarized light. The optical activity, or direction of rotation of plane-polarized light, of an enantiomer of a compound of the invention may be determined using conventional techniques.

The compounds of the present invention may be in racemic mixture, enantiomerically enriched or enantiomerically pure form. The terms “racemate” and “racemic mixture” as used herein refer to a mixture of the (R)— and the (S)— optical isomers (e.g., enantiomers) of a compound in equal, i.e. 50:50 proportion.

The term “enantiomerically enriched” as used herein refers to preparations comprising a mixture of optical isomers in which the quantity of one enantiomer is higher than the quantity of the other. Thus, “enantiomerically enriched” refers to mixtures of optical isomers wherein the ratio of enantiomer is greater than 50:50. An enantiomerically enriched compound comprises greater than 50% by weight of one enantiomer relative to the other. For example enantiomerically enriched 5-[5,6-Bis(methyloxy)-1H-benzimidazol-1-yl]-3-({(1R)-1-[2-chloro-5-({[2-(dimethylamino)ethyl]amino}carbonyl)-phenyl]ethyl}oxy)-2-thiophenecarboxamide formate, refers to a composition comprising greater than 50% by weight of the (R)-enantiomer relative to the (S)-enantiomer of the compound. In one embodiment, an enantiomerically enriched compound comprises at least 75% by weight of one enantiomer relative to the other. In another embodiment, an enantiomerically enriched compound comprises at least 80% by weight of one enantiomer relative to the other. In one particular embodiment, an enantiomerically enriched compound comprises at least 85% by weight of one enantiomer relative to the other.

The term “enantiomerically pure” as used herein refers to enantiomerically enriched compounds comprising at least 90% by weight of one enantiomer relative to the other. In one embodiment, an enantiomerically pure compound comprises at least 95% by weight of one enantiomer relative to the other. In one particular embodiment, an enantiomerically pure compound comprises at least 99% by weight of one enantiomer relative to the other.

In one embodiment, the present invention provides an enantiomerically enriched compound of formula (I), having the stereochemistry depicted in formula (I-1):

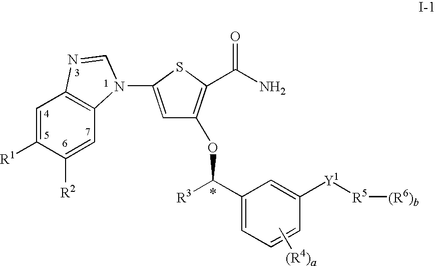

wherein * indicates the chiral carbon and all variables are as defined above. The foregoing specific embodiments of the invention described above for the variables defining compounds of formula (I) are equally applicable to compounds of formula (I-1).

It is to be understood that the present invention includes all combinations and subsets of the particular groups defined hereinabove.

Specific examples of compounds within the scope of the present invention include those recited in the Examples which follow and pharmaceutically acceptable salts and solvates thereof.

It will be appreciated by those skilled in the art that the compounds of the present invention may be utilized not only in the form of the free base, but also in the form of a pharmaceutically acceptable salt or solvate thereof. The pharmaceutically acceptable salts of the compounds of the present invention (or the enantiomerically enriched or pure forms thereof) include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, trifluoroacetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic (mesylate), naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

The term “solvate” as used herein refers to a complex of variable stoichiometry formed by a solute (a compound of the invention or an enaniomerically enriched or pure form thereof) and a solvent. Solvents, by way of example, include water, methanol, ethanol, or acetic acid.

Processes for preparing pharmaceutically acceptable salts and solvates of the compounds of the invention are conventional in the art. See, e.g., Burger's Medicinal Chemistry And Drug Discovery 5th Edition, Vol 1: Principles And Practice.

As will be apparent to those skilled in the art, in the processes described below for the preparation of the compounds of the invention, certain intermediates, may alternatively be in the form of pharmaceutically acceptable salts or solvates of the compound. Those terms as applied to any intermediate employed in the process of preparing the compounds of the invention have the same meanings as noted above with respect to the compounds of the invention. Processes for preparing pharmaceutically acceptable salts and solvates of such intermediates are known in the art and are analogous to the process for preparing pharmaceutically acceptable salts and solvates of the compounds of the invention.

The compounds of the present invention are typically inhibitors of PLK, in particular, PLK1. By PLK inhibitor is meant a compound which exhibits pIC₅₀ greater than 6 in the PLK Inhibition assay described below in the examples or an IC₅₀ less than 10 μM in the Cell-Titer Glo or Methylene Blue Cell Growth Inhibition assays described below in the examples; more particularly a PLK inhibitor is a compound which exhibits a pIC₅₀ greater than 7 in the PLK Inhibition assay or an IC₅₀ less than 1 μM in the Cell-Titer Glo or Methylene Blue Cell Growth Inhibition assay using the methods described in the examples below.

The present invention further provides compounds of the invention for use in medical therapy in an animal, e.g. a mammal such as a human. In particular, the present invention provides compounds for use in the treatment of a condition mediated by PLK, particularly PLK1. The present invention also provides compounds for use in the treatment of a susceptible neoplasm. In particular, the present invention provides compounds for use in the treatment of a variety of solid tumors including but not limited to breast cancer, ovarian cancer, non-small cell lung cancer and prostate cancer as well as hematologic malignancies including but not limited to acute leukemias and aggressive lymphomas. “Acute leukemias” includes both acute myeloid leukemias and acute lymphoid leukemias. See, N. Harris, et al., J. Clin. One. (1999) 17(12):3835-3849. “Aggressive lymphomas” is a term of art. See, J. Chan, Hematological Onc. (2001) 19:129-150.

The present invention provides compounds for use in treating a condition characterized by inappropriate cellular proliferation. The present invention also provides compounds for use in inhibiting proliferation of a cell. The present invention also provides compounds for use in inhibiting mitosis in a cell.

The present invention provides methods for the treatment of several conditions or diseases, all of which comprise the step of administering a therapeutically effective amount of a compound of the invention. As used herein, the term “treatment” refers to alleviating the specified condition, eliminating or reducing the symptoms of the condition, slowing or eliminating the progression of the condition and preventing or delaying the reoccurrence of the condition in a previously afflicted subject.

As used herein, the term “therapeutically effective amount” means an amount of a compound of the invention which is sufficient, in the subject to which it is administered, to elicit the biological or medical response of a cell culture, tissue, system, animal (including human) that is being sought, for instance, by a researcher or clinician. For example, a therapeutically effective amount of a compound of the invention for the treatment of a condition mediated by PLK, particularly PLK1, is an amount sufficient to treat the PLK mediated condition in the subject. Similarly, a therapeutically effective amount of a compound of the invention for the treatment of a susceptible neoplasm is an amount sufficient to treat the susceptible neoplasm in the subject. In one embodiment of the present invention, the therapeutically effective amount of a compound of the invention is an amount sufficient to treat breast cancer in a human in need thereof. In one embodiment of the present invention, a therapeutically effective amount of a compound of the invention is an amount sufficient to regulate, modulate, bind or inhibit PLK, particularly PLK1.

The precise therapeutically effective amount of the compounds of the invention will depend on a number of factors including, but not limited to, the age and weight of the subject being treated, the precise condition or disease requiring treatment and its severity, the nature of the formulation, and the route of administration, and will ultimately be at the discretion of the attendant physician or veternarian. Typically, the compound of the invention will be given for treatment in the range of 0.1 to 200 mg/kg body weight of recipient (animal) per day, per dose or per cycle of treatment and more usually in the range of 1 to 100 mg/kg body weight per day, per dose or per cycle of treatment. Acceptable daily dosages, may be from about 0.1 to about 2000 mg per day, per dose or per cycle of treatment, and preferably from about 0.1 to about 500 mg per day, per dose or per cycle of treatment.

As one aspect, the present invention provides methods of regulating, modulating, binding, or inhibiting PLK for the treatment of conditions mediated by PLK, particularly PLK1. “Regulating, modulating, binding or inhibiting PLK” refers to regulating, modulating, binding or inhibiting PLK, particularly PLK1 activity, as well as regulating, modulating, binding or inhibiting overexpression of PLK, particularly PLK1. Such conditions include certain neoplasms (including cancers and tumors) which have been associated with PLK, particularly PLK1, and conditions characterized by inappropriate cellular proliferation.

The present invention provides a method for treating a condition mediated by PLK, particularly PLK1 which comprises administering to the animal a therapeutically effective amount of the compound of the invention. This method and other methods of the present invention are useful for the treatment of an animal such as a mammal and in particular humans. Conditions which are mediated by PLK are known in the art and include but are not limited to neoplasms and conditions characterized by inappropriate cellular proliferation.

The present invention also provides a method for treating a susceptible neoplasm (cancer or tumor) in an animal such as a mammal (e.g., a human) in need thereof, which method comprises administering to the animal a therapeutically effective amount of the compound of the invention. “Susceptible neoplasm” as used herein refers to neoplasms which are susceptible to treatment with a PLK, particularly PLK1, inhibitor. Neoplasms which have been associated with PLK and are therefore susceptible to treatment with a PLK inhibitor are known in the art, and include both primary and metastatic tumors and cancers. See e.g., M. Whitfield et al., (2006) Nature Reviews/Cancer 6:99. For example, susceptible neoplasms within the scope of the present invention include but are not limited to breast cancer, colon cancer, lung cancer (including small cell lung cancer and non-small cell lung cancer), prostate cancer, endometrial cancer, gastric cancer, melanoma, ovarian cancer, pancreatic cancer, squamous cell carcinoma, carcinoma of the head and neck, esophageal carcinoma, hepatocellular carcinoma and hematologic malignancies such as acute leukemias and aggressive lymphomas. In one particular embodiment, the present invention provides a method of treating breast cancer in an animal, such as a mammal (e.g., a human) in need thereof by administering a therapeutically effective amount of a compound of the present invention. In another particular embodiment, the present invention provides a method of treating ovarian cancer in an animal, such as a mammal (e.g., a human) in need thereof by administering a therapeutically effective amount of a compound of the present invention. In another particular embodiment, the present invention provides a method of treating non-small cell lung cancer in an animal, such as a mammal (e.g., a human) in need thereof by administering a therapeutically effective amount of a compound of the present invention. In another particular embodiment, the present invention provides a method of treating prostate cancer in an animal, such as a mammal (e.g., a human) in need thereof by administering a therapeutically effective amount of a compound of the present invention.

In another particular embodiment, the present invention provides a method of treating hematologic malignancies including acute leukemias and aggressive lymphomas in an animal, such as a mammal (e.g., a human) in need thereof by administering a therapeutically effective amount of a compound of the present invention.

The compounds of the invention can be used alone in the treatment of such susceptible neoplasms or can be used to provide additive or synergistic effects with one or more other compounds of the invention, or in combination with certain existing chemotherapies and/or other anti-neoplastic therapies. In addition, the compounds of the invention can be used to restore effectiveness of certain existing chemotherapies and/or other anti-neoplastic therapies. As used herein, “anti-neoplastic therapies” includes but is not limited to cytotoxic chemotherapy, hormonal therapy, targeted kinase inhibitors, therapeutic monoclonal antibodies, surgery and radiation therapy.

The present invention also provides a method for treating a condition characterized by inappropriate cellular proliferation in an animal, such as a mammal (e.g., a human) in need thereof. The method comprises administering a therapeutically effective amount of a compound of the present invention. By “inappropriate cellular proliferation” is meant cellular proliferation resulting from inappropriate cell growth, cellular proliferation resulting from excessive cell division, cellular proliferation resulting from cell division at an accelerated rate, cellular proliferation resulting from inappropriate cell survival, and/or cellular proliferation in a normal cell occurring at a normal rate, which is nevertheless undesired. Conditions characterized by inappropriate cellular proliferation include but are not limited to neoplasms, blood vessel proliferative disorders, fibrotic disorders, mesangial cell proliferative disorders and inflammatory/immune-mediated diseases. Blood vessel proliferative disorders include arthritis and restenosis. Fibrotic disorders include hepatic cirrhosis and atherosclerosis. Mesangial cell proliferative disorders include glomerulonephritis, malignant nephrosclerosis and glomerulopathies. Inflammatory/immune-mediated disorders include psoriasis, chronic wound healing, organ transplant rejection, thrombotic microangiopathy syndromes, and neurodegenerative diseases. Osteoarthritis and other osteoclast proliferation dependent diseases of excess bone resorbtion are examples of conditions characterized by inappropriate cellular proliferation in which the cellular proliferation occurs in normal cells at a normal rate, but is nevertheless undesired.

The present invention also provides a method for inhibiting proliferation of a cell, which method comprises contacting the cell with an amount of a compound of the invention sufficient to inhibit proliferation of the cell. In one particular embodiment, the cell is a neoplastic cell. In one particular embodiment, the cell is an inappropriately proliferative cell. The term “inappropriately proliferative cell” as used herein refers to cells that grow inappropriately (abnormally), cells that divide excessively or at an accelerated rate, cells that inappropriately (abnormally) survive and/or normal cells that proliferate at a normal rate but for which proliferation is undesired. Neoplastic cells (including cancer cells) are an example of inappropriately proliferative cells but are not the only inappropriately proliferative cells.

PLK is essential for cellular mitosis and accordingly, the compounds of the invention are believed to be effective for inhibiting mitosis. “Inhibiting mitosis” refers to inhibiting the entry into the M phase of the cell cycle, inhibiting the normal progression of the M phase of the cell cycle once M phase has been entered and inhibiting the normal exit from the M phase of the cell cycle. Thus, the compounds of the present invention may inhibit mitosis by inhibiting the cell's entry into mitosis, by inhibiting the cell's progression through mitosis or by inhibiting the cell's exit from mitosis. As one aspect, the present invention provides a method for inhibiting mitosis in a cell, which method comprises administering to the cell an amount of a compound of the invention sufficient to inhibit mitosis. In one particular embodiment, the cell is a neoplastic cell. In one particular embodiment, the cell is an inappropriately proliferative cell.

The present invention also provides the use of a compound of the invention for the preparation of a medicament for the treatment of condition mediated by PLK, particularly PLK1, in an animal, such as a mammal (e.g., a human). The present invention further provides the use of a compound for the preparation of a medicament for the treatment of a susceptible neoplasm in an animal, particularly a mammal (e.g., a human). In particular, the present invention provides the use of a compound for the preparation of a medicament for the treatment of a breast cancer. The present invention also provides the use of a compound for the preparation of a medicament for the treatment of ovarian cancer. The present invention provides the use of a compound for the preparation of a medicament for the treatment of non-small cell lung cancer. The present invention provides the use of a compound for the preparation of a medicament for the treatment of prostate cancer. The present invention provides the use of a compound for the preparation of a medicament for the treatment of hematologic malignancies such as acute leukemias and aggressive lymphomas. The present invention further provides the use of a compound for the preparation of a medicament for the treatment of a condition characterized by inappropriate cellular proliferation. The present invention further provides the use of a compound for the preparation of a medicament for inhibiting proliferation of a cell. The present invention further provides the use of a compound for the preparation of a medicament for inhibiting mitosis in a cell.

While it is possible that, for use in therapy, a therapeutically effective amount of a compound of the invention may be administered as the raw chemical, it is typically presented as the active ingredient of a pharmaceutical composition or formulation. Accordingly, the invention further provides a pharmaceutical composition comprising a compound of the invention. The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the invention with one or more pharmaceutically acceptable carriers, diluents and/or excipients.

Pharmaceutical formulations may be presented in unit dose form containing a predetermined amount of active ingredient per unit dose. Such a unit may contain a therapeutically effective dose of the compound of the invention or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quarternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be delivered by the use of monoclonal antibodies as individual carriers to which the compound molecules are coupled. The compounds may also be coupled with soluble polymers as targetable drug carriers. Such polymers can include peptides, polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986). Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream.

When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators. Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

In the above-described methods of treatment and uses, a compound of the invention may be employed alone, in combination with one or more other compounds of the invention or in combination with other therapeutic agents and/or in combination with other anti-neoplastic therapies. In particular, in methods of treating conditions mediated by PLK and methods of treating susceptible neoplasms, combination with other chemotherapeutic agents is envisaged as well as combination with surgical therapy and radiation therapy. The term “chemotherapeutic” as used herein refers to any chemical agent having a therapeutic effect on the subject to which it is administered. “Chemotherapeutic” agents include but are not limited to anti-neoplastic agents, analgesics and anti-emetics. As used herein, “anti-neoplastic agents” include both cytostatic and cytotoxic agents such as but not limited to cytotoxic chemotherapy, hormonal therapy, targeted kinase inhibitors and therapeutic monoclonal antibodies. Combination therapies according to the present invention thus comprise the administration of at least one compound of the invention and the use of at least one other cancer treatment method. In one embodiment, combination therapies according to the present invention comprise the administration of at least one compound of the invention and at least one other chemotherapeutic agent. In one particular embodiment, the present invention comprises the administration of at least one compound of the invention and at least one anti-neoplastic agent. As an additional aspect, the present invention provides the methods of treatment and uses as described above, which comprise administering a compound of the invention together with at least one chemotherapeutic agent. In one particular embodiment, the chemotherapeutic agent is an anti-neoplastic agent. In another embodiment, the present invention provides a pharmaceutical composition as described above further comprising at least one other chemotherapeutic agent, more particularly, the chemotherapeutic agent is an anti-neoplastic agent.

Typically, any chemotherapeutic agent that has activity versus a susceptible neoplasm being treated may be utilized in combination with the compounds of the invention, provided that the particular agent is clinically compatible with therapy employing a compound of the invention. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids. Examples of diterpenoids include, but are not limited to, paclitaxel and its analog docetaxel. Examples of vinca alkaloids include, but are not limited to, vinblastine, vincristine, and vinorelbine.

Platinum coordination complexes are non-phase specific anti-neoplastic agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Antibiotic chemotherapeutic agents are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthracyclins such as daunorubicin and doxorubicin; and bleomycins.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine and thioguanine.

Camptothecins, including, camptothecin and camptothecin derivatives are available or under development as Topoisomerase I inhibitors.

Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin.

Hormones and hormonal analogues are useful compounds for treating cancers in which there is a relationship between the hormone(s) and growth and/or lack of growth of the cancer. Examples of hormones and hormonal analogues believed to be useful in the treatment of neoplasms include, but are not limited to, adrenocorti-costeroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene and iodoxyfene useful in the treatment of hormone dependent breast carcinoma; and gonadotropin-releasing hormone (GnRH) and analogues thereof, such as goserelin acetate and leuprolide, which stimulate the release of leutinizing hormone (LH) and/or follicle stimulating hormone (FSH) with short-term or intermittent use but lead to suppression of LH and FSH with long-term use indicated for the treatment prostatic carcinoma, and hormone dependent breast carcinoma.

Signal transduction pathway inhibitors are those inhibitors which block or inhibit a chemical process which evokes an intracellular change. As used herein this change is cell proliferation, survival, angiogenesis or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr, ErbB2 and ErbB4,), platelet derived growth factor receptor (PDGFr), vascular endothelial growth factor receptor (VEGFR), tyrosine kinase with immunoglobulin-like and epidermal growth factor homology domains (TIE-2), insulin growth factor-I receptor (IGF-1), macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth factor receptors are under development and include ligand antagonists, antibodies, tyrosine kinase inhibitors, anti-sense oligonucleotides and aptamers. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth Factor Receptors as Targets”, New Molecular Targets for Cancer Chemotherapy, Ed. Workman, Paul and Kerr, David, CRC Press 1994, London.

Tyrosine kinases, which are not growth factor receptor kinases are termed non-receptor tyrosine kinases. Non-receptor tyrosine kinases useful in the present invention, which are targets or potential targets of anti-neoplastic drugs, include cSrc, Lck, Fyn, Yes, Jak, cAbI, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual Review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (Rafk), Mitogen or Extracellular Regulated Kinase (MEKs), and Extracellular Regulated Kinases (ERKs); and Protein kinase C family member blockers including blockers of subtypes of PKCs (alpha, beta, gamma, epsilon, mu, lambda, iota, zeta), IkB kinase family (IKKa, IKKb), PKB family kinases, Akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; and Martinez-Iacaci, L., et al, Int. J. Cancer (2000), 88(1), 44-52.

Inhibitors of Phosphotidyl Inositol-3 Kinase family members including blockers of PI3-kinase, ATM, DNA-PK, and Ku are also useful in combination with the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer Res, (2000) 60(6), 1541-1545.

Also useful in combination with the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC Press 1994, London.

Another group of signal transduction pathway inhibitors useful in combination with the present invention are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block Ras activation in cells containing wild type mutant Ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9(2)99-102; and BioChim. Biophys. Acta, (1989) 1423(3):19-30.

As mentioned above, antibodies to receptor kinase ligand binding may also serve as signal transduction inhibitors. This group of signal transduction pathway inhibitors includes the use of humanized antibodies to the extracellular ligand binding domain of receptor tyrosine kinases. For example, Imclone C225 EGFR specific antibody (see Green, M. C. et al, Monoclonal Antibody Therapy for Solid Tumors, Cancer Treat. Rev., (2000), 26(4), 269-286); Herceptin® ErbB2 antibody (see Tyrosine Kinase Signaling in Breast Cancer: ErbB Family Receptor Tyrosine Kinases, Breast Cancer Res., 2000, 2(3), 176-183); and 2CB VEGFR2 specific antibody (see Brekken, R. A. et al, Selective Inhibition of VEGFR2Activity by a Monoclonal Anti-VEGF Antibody Blocks Tumor Growth in Mice, Cancer Res. (2000) 60, 5117-5124).

Receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Other inhibitors may be used in combination with the compounds of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with PLK inhibitors.

Agents used in immunotherapeutic regimens may also be useful in combination with the compounds of the invention.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al., J. Clin. Oncol. 18:1812-1823 (2000); and Kitada S et al., Antisense Res. Dev. 4:71-79 (1994).

Cell cycle signaling inhibitors inhibit molecules involved in the control of the cell cycle. Cyclin dependent kinases (CDKs) and their interaction cyclins control progression through the eukaryotic cell cycle. The coordinated activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, Rosania, et al., Exp. Opin. Ther. Patents 10(2):215-230 (2000).

In one embodiment, the methods of the present invention comprise administering to the animal a compound of the invention in combination with a signal transduction pathway inhibitor, particularly gefitinib (IRESSA®).

The methods and uses employing these combinations may comprise the administration of the compound of the invention and the other chemotherapeutic/anti-neoplastic agent either sequentially in any order or simultaneously in separate or combined pharmaceutical compositions. When combined in the same formulation it will be appreciated that the two compounds must be stable and compatible with each other and the other components of the formulation and may be formulated for administration. When formulated separately they may be provided in any convenient formulation, in such a manner as are known for such compounds in the art.

When a compound of the invention is used in combination with a chemotherapeutic agent, the dose of each compound may differ from that when the compound is used alone. Appropriate doses will be readily appreciated by those skilled in the art. The appropriate dose of the compound(s) of the invention and the other therapeutically active agent(s) and the relative timings of administration will be selected in order to achieve the desired combined therapeutic effect, and are within the expertise and discretion of the attendent clinician.

The compounds of the invention may be conveniently prepared by the process outlined in Scheme 1 below.

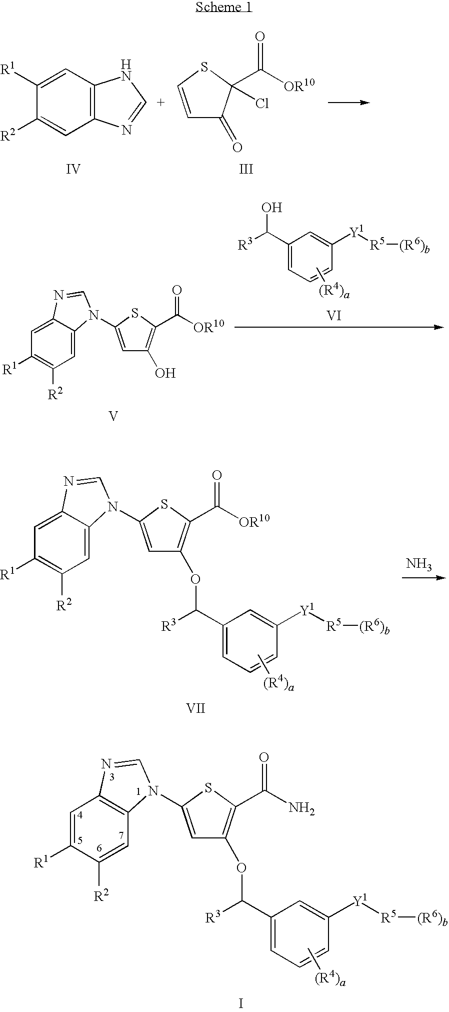

wherein:
Y¹ is —O—;
R¹⁰ is selected alkyl and suitable carboxylic acid protecting groups; and all other variables are as defined above.

Generally, the process for preparing the compounds of the invention (all formulas and all variables having been defined above) comprises the steps of:
a) reacting the compound of formula (IV) with a compound of formula (III) to prepare a compound of formula (V);
b) reacting the compound of formula (V) with a compound of formula (VI) to prepare a compound of formula (VII);
c) reacting the compound of formula (VII) with ammonia to prepare a compound of formula (I);
d) optionally separating the compound of formula (I) into enantiomers of formula (I);
e) optionally converting the compound of formula (I) to a pharmaceutically acceptable salt or solvate thereof; and
f) optionally converting the compound of formula (I) or a pharmaceutically acceptable salt or solvate thereof to a different compound of formula (I) or a pharmaceutically acceptable salt or solvate thereof.

As will be apparent to those skilled in the art, the order of the steps in the foregoing reaction is not critical to the practice of the process of the present invention. The foregoing reaction steps may be carried out in any suitable order based upon the knowledge of those skilled in the art. Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art.

More specifically, compounds of the invention can be prepared by reacting a compound of formula (VII) with ammonia to prepare a compound of formula (I).

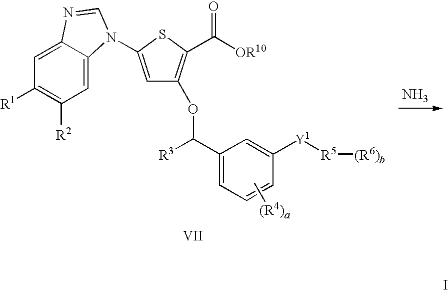

wherein all variables are as defined above.

This reaction is typically performed in a sealed vessel with an excess of ammonia. The reaction is typically heated to a temperature of from about 50 to about 120° C., more particularly, about 70° C. Suitable solvents for this reaction include but are not limited to methanol, ethanol, isopropanol, tetrahydrofuran, and dioxane.

A compound of formula (I) may be separated, using conventional separation techniques (e.g., supercritical fluid chromatography (SCF)) into its enantiomers, the enantiomerically enriched compounds of formula (I-1) and (I-2).

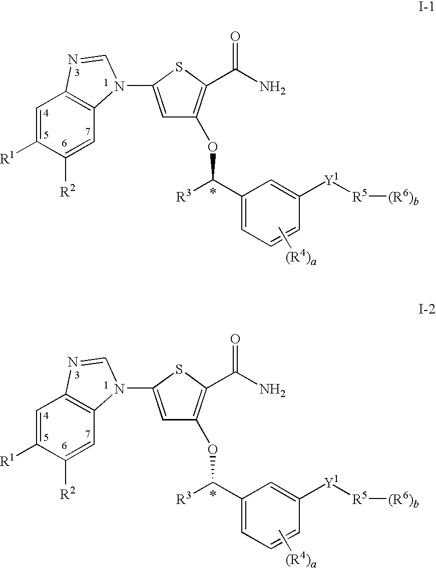

wherein * indicates the chiral carbon and all variables are as defined above.

A compound of formula (VII) may be prepared by reacting a compound of formula (V) with a compound of formula (VI) under Mitsunobu reaction conditions.

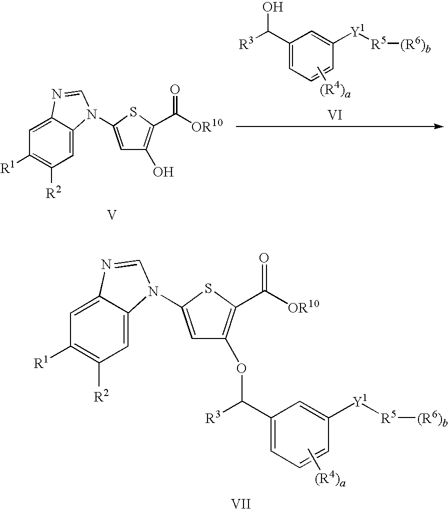

wherein all variables are as defined above.

The reaction is carried out in an inert solvent under standard Mitsunobu conditions. See, Hughes, D. L., Org. React. 42:335-656 (1992); and Mitsunobu, O., Synthesis 1-28 (1981). Typically the compound of formula (V), the compound of formula (VI), a triarylphosphine, and a dialkyl azodicarboxylate are reacted together at room temperature. Examples of suitable triarylphosphines include but are not limited to, triphenylphosphine, tri-tolylphosphine, and trimesitylphosphine. Examples of suitable dialkyl azodicarboxylates include but are not limited to, diethyl azodicarboxylate, diisopropyl azodicarboxylate, and di-tert-butyl azodicarboxylate. Examples of suitable inert solvents for this reaction include but are not limited to, tetrahydrofuran, dioxane, 1,2-dimethoxyethane, dichloromethane, and toluene.

If desired, the compound of formula (VII) may be separated using conventional separation techniques (e.g., SFC) into its enantiomers, enantiomerically enriched compounds of formula (VII-1) and (VII-2).

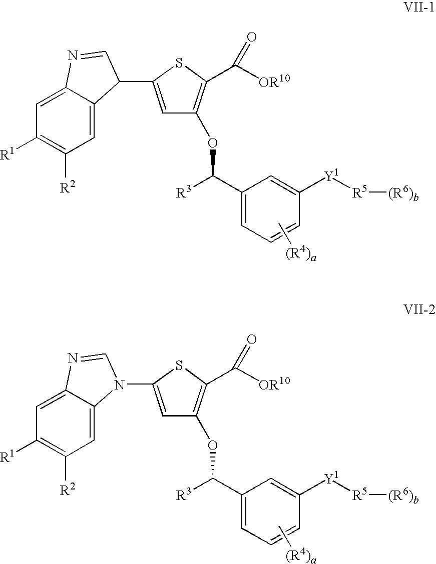

As will be apparent to those skilled in the art, reaction of an enantiomerically enriched compound of formula (VII-1) or (VII-2) with ammonia will result in the corresponding enantiomerically enriched compound of formula (I-1) or (I-2), respectively.

The compounds of formula (VI) may be prepared by reducing a compound of formula (XI). The compounds of formula (XI) may be prepared by reacting a compound of formula (IX) with a compound of formula (X) under Mitsunobu reaction conditions.

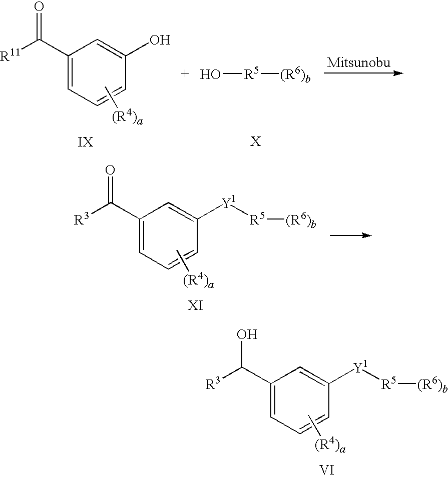

wherein:
Y¹ is —O—;
R¹¹ is H or R³; and
   all variables are as defined above.

Suitable Mitsunobu reaction conditions and solvents are described above. The Mitsunobu reaction yields a compound of formula (XI).

Compounds of formula (XI), where R¹¹ is H, may be reacted with R³—Li (alkyl lithium) or R³—MgCl (alkyl magnesium chloride) to prepare a compound of formula (VI). In one embodiment, the compounds of formula (XI), where R¹¹ is H, may be reacted with methyl lithium in the presence of titanium (VI) chloride, or methyl magnesium chloride to prepare a compound of formula (VI) where R³ is methyl. The reaction typically can be carried out in an inert atmosphere. The suitable solvents may include ether and tetrahydrofuran. The reaction temperature may be in the range of −78° C. to room temperature.

Compounds of formula (XI) may also be reacted with reducing agents such as borane, lithium hydride or sodium borohydrate to prepare a compound of formula (VI). Suitable techniques for conversion of an aldehyde or ketone to an alcohol are well known to those skilled in the art. See, Larock, R. Comprehensive Organic Transformation (2nd Edition), John Wiley & Sons, Inc. (1999) 1075-1077.

In one embodiment, the compound of formula (XI) is reacted with borane/dimethylsulfide complex in tetrahydrofuran and (R)-1-methyl-3,3-diphenyltetrahydro-3H-pyrrolo[1,2-c][1,3,2]oxazaborole in a solvent such as toluene to prepare an enantiomerically enriched compound of formula (VI) having the stereochemistry depicted in formula (VI-1):

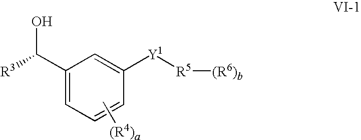

wherein all variables are as defined above.

As will be apparent to those skilled in the art, use of the enantiomerically enriched compound of formula (VI-1) in the reaction with the compound of formula (V) will yield an enantiomerically enriched compound of formula (VII-1) which may be reacted with ammonia to yield the enantiomerically enriched compound of formula (I-1).

The compounds of formula (V) may be prepared by reacting a compound of formula (IV) with a compound of formula (III).

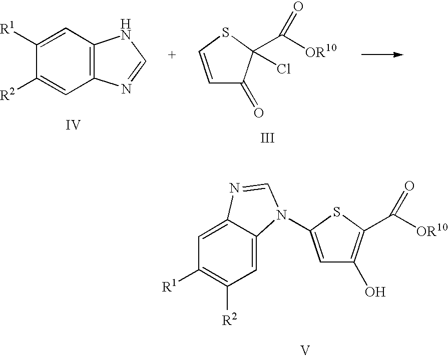

wherein all variables are as defined above.

Processes for the reaction of a compound of formula (IV) with a compound of formula (III) are known to those skilled in the art. See, PCT Int. Appl. WO 2004073612. Such reactions are typically carried out in an inert solvent at room temperature. Examples of suitable inert solvents for this reaction include but are not limited to, chloroform, dichloromethane, tetrahydrofuran, dioxane, and toluene and mixtures of any of the foregoing with acetic acid (e.g., a mixture of chloroform and acetic acid). In one embodiment, the inert solvent is selected from dichloromethane, chloroform, tetrahydrofuran, diethyl ether, and toluene and a mixture of any of the foregoing and acetic acid (e.g. a mixture of chloroform and acetic acid).

The reaction may be carried out in the presence of one to five equivalents of the base additive. The base additive is believed to act as a scavenger for the hydrochloric acid generated during the reaction. Examples of suitable base additives for this reaction include but are not limited to sodium bicarbonate, triethylamine, sodium acetate, N-methylimidazole, pyridine, N-methylbenzimidazole and potassium carbonate. In one embodiment, the base additive is selected from sodium bicarbonate, triethylamine, sodium acetate, N-methylimidazole, pyridine and N-methylbenzimidazole. In one particular embodiment, the base additive is sodium bicarbonate. In one particular embodiment, the base additive is N-methylimidazole.

Compounds of formula (IV) may be prepared by a process depicted below:

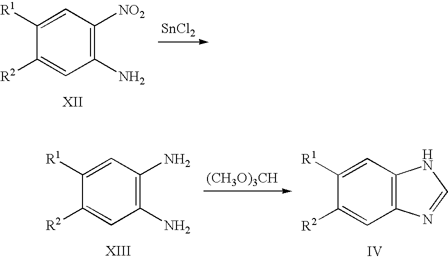

wherein all variables are as defined above.

This process comprises the steps of:
a) reducing a 2-nitroaniline of formula (XII) to prepare a substituted 1,2-diamine of formula (XIII); and
b) cyclizing the 1,2-diamine of formula (XIII) with a ring forming reagent, such as trimethylorthoformate, to prepare compounds of formula (IV).

The ring forming reaction may be carried out using conventional techniques. See, White, A., et al., J. Med. Chem. 43:4084-4097 (2000); Jiang, J.-L., et al., Synthetic Comm. 28:4137-4142 (1998); Tanaka, A., et al., Chem. Pharm. Bull. 42:560-569 (1994); Tian, W., et al., Synthesis 12:1283-1286 (1992); Buckle, D. R., et al., J. Med. Chem. 30:2216-2221 (1987); and Raban, M., et al., J. Org. Chem. 50:2205-2210 (1985). This reaction may be carried out neat or in a suitable solvent. The reaction may optionally be heated to a temperature of from about 50 to about 230° C. The reaction is typically carried out with an excess of trimethylorthoformate. An additional acid may be used. Examples of suitable acids include but are not limited to, formic acid, hydrochloric acid, hydrobromic acid, perchloric acid, sulfuric acid, R-toluenesulfonic acid, methanesulfonic acid, and trifluoromethanesulfonic acid. Examples of suitable solvents for this reaction include but are not limited to water, methanol, ethanol, isopropanol, tetrahydrofuran, dichloromethane, toluene, N,N-dimethylformamide, dimethylsulfoxide, and acetonitrile.

The reduction of the 2-nitroaniline of formula (XII) may be carried out using conventional techniques and reducing agents such as tin(II) chloride. See, Rangarajan, M., et al., Bioorg. Med. Chem. 8:2591-2600 (2000); White, A. W., et al., J. Med. Chem. 43: 4084-4097 (2000); Silvestri, R., et al., Bioorg. Med. Chem. 8:2305-2309 (2000); Nagaraja, D., et al., Tetrahedron Lett. 40:7855-7856 (1999); Jung, F., et al., J. Med. Chem. 34:1110-1116 (1991); Srivastava, R. P., et al., Pharmazie 45:34-37 (1990); Hankovszky, H. O., et al., Can. J. Chem. 67:1392-1400 (1989); Ladd, D. L., et al., J. Org. Chem. 53:417-420 (1988); Mertens, A., et al., J. Med. Chem. 30:1279-1287 (1987); and Sharma, K. S., et al., Synthesis 4:316-318 (1981). Examples of other suitable reducing agents for this reaction include but are not limited to, palladium with hydrogen, palladium with ammonium formate, platinum oxide with hydrogen, nickel with hydrogen, iron with acetic acid, aluminum with ammonium chloride, borane, sodium dithionite, and hydrazine. The reaction may optionally be heated to between about 50 and about 120° C. Suitable solvents for this reaction vary and include but are not limited to, water, methanol, ethanol, ethyl acetate, tetrahydrofuran, dioxane, and mixtures thereof.

Compounds of formula (III) may be prepared by reacting a compound of formula (II) with sulfuryl chloride.

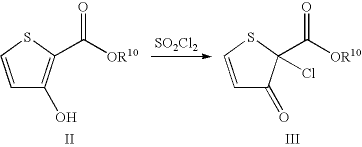

wherein all variables are as defined above.

Compounds of formula (II) are commercially available or can be prepared using conventional techniques. Typically the reaction is carried out at room temperature. Excess sulfuryl chloride may be used if desired. Examples of suitable solvents include but are not limited to chloroform, dichloromethane, and toluene. See, Corral, C.; Lissavetzky, J. Synthesis 847-850 (1984).

In another embodiment, a compound of formula (V) may be prepared according to the process of Scheme 2:

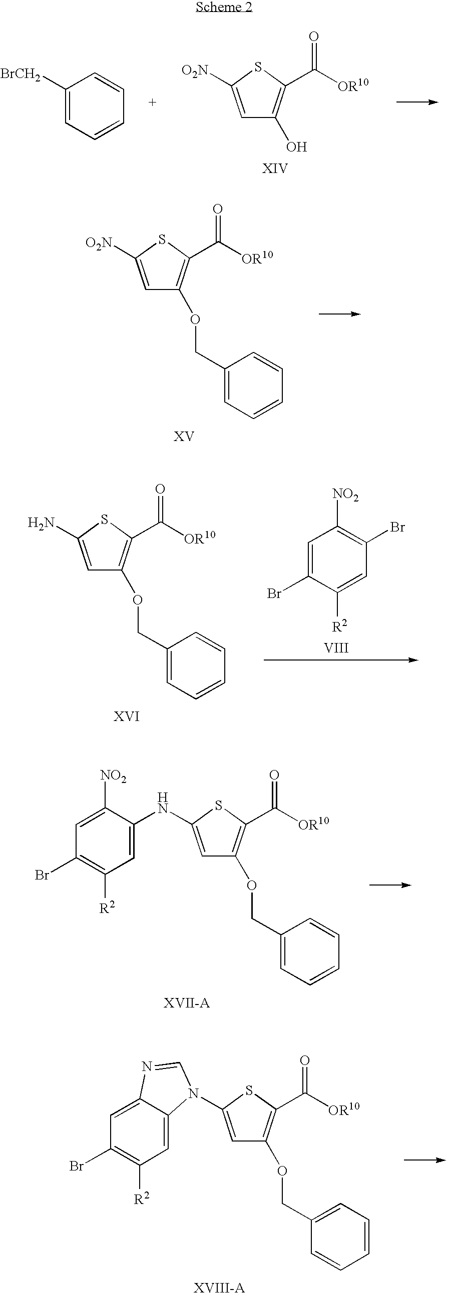

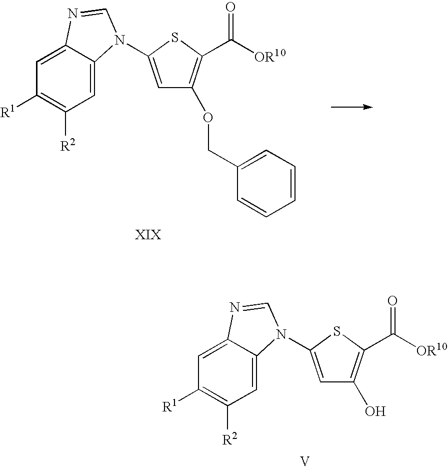

wherein:
R¹⁰ is selected from alkyl and suitable carboxylic acid protecting groups;
Y¹ is —O—; and
   all other variables are as defined above.

Generally, the process for preparing the compounds of formula (V) (all formulas and all variables having been defined above) comprises the steps of:
a) reacting a compound of formula (XIV) with a protecting group, such as benzyl bromide, to prepare a compound of formula (XV);
b) reducing the compound of formula (XV) to prepare a compound of formula (XVI);
c) reacting the compound of formula (XVI) with 1,4-dibromo-2-nitrobenzene of formula (VII) to prepare a compound of formula (XVII-A);
d) reducing and cyclizing the compound of formula (XVII-A) to prepare a compound of formula (XVIII-A);
e) reacting the compound of formula (XVIII-A) under conventional cross-coupling reaction conditions to prepare a compound of formula (XIX);
f) reacting the compound of formula (XIX) with acid to prepare a compound of formula (V).

According to this process a compound of formula (V) is prepared by reacting a compound of formula (XIX) with a suitable acid, such as trifluoroacetic acid or hydrochloric acid.

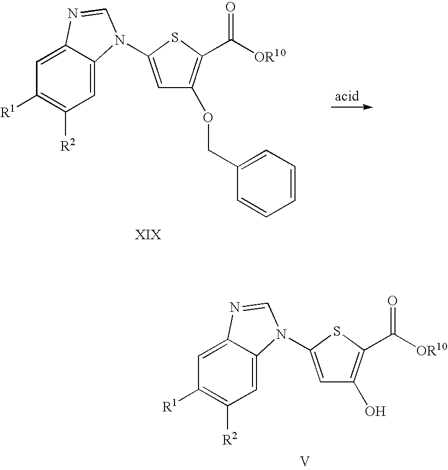

This reaction may be carried out in neat trifluoroacetic acid or in an inert solvent such as dichloromethane at ambient temperature.

The compound of formula (XIX) may be prepared by reacting a compound of formula (XVIII-A) under conventional cross-coupling reaction conditions.

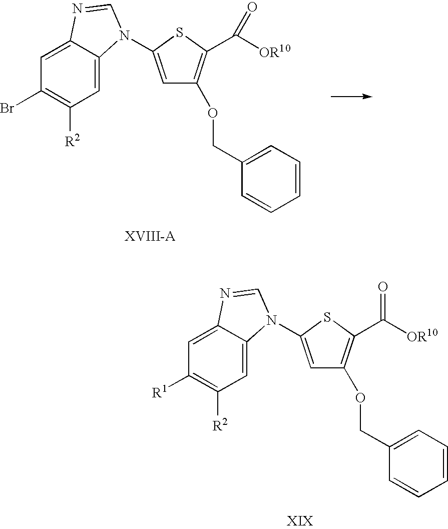

wherein all variables are as defined above.

In particular, a compound of formula (XIX) may be prepared from a compound of formula (XVIII-A) using palladium-catalyzed Suzuki, Stille, or Negishi cross-coupling techniques conventional in the art of organic synthesis. For a review of the Suzuki cross-coupling reaction, see: Miyaura, N.; Suzuki, A. Chemical Reviews 1995, 95, 2457-2483. The Suzuki coupling may be carried out using a suitable catalyst such as dichloro[1,1′-bis(diphenylphosphino)ferrocene] palladium(II) dichloromethane adduct, a base such as aqueous sodium carbonate or triethylamine, and a suitable inert solvent such as N,N-dimethylacetamide or n-propanol, optionally in the presence of microwave irradiation, at temperatures from about 50° C. to about 150° C. For a review of the Stille cross-coupling reaction, see: Mitchell, T. N. Synthesis 1992, 803-815. The Stille coupling may be carried out using tetrakis(triphenylphoshine)-palladium (0) as the catalyst, in the presence of promoters such as cesium fluoride and copper (I) iodide, in a suitable inert solvent such as N,N-dimethylformamide at a temperature of about 45° C. For a review of the Negishi cross-coupling reaction, see: Negishi, E.; Zingzhong, T. Z.; Qian, M.; Hu, Q.; Huang, Z. Metal Catalyzed Cross-Coupling Reactions (2^{nd} Edition), 2004, 2, 815-889. The Negishi coupling may be carried out using dichloro[1,1′-bis(diphenylphosphino)-ferrocene] palladium(II) dichloromethane adduct as the catalyst, in the presence of a promoter such as copper (I) iodide, in a suitable inert solvent such as N,N-dimethylacetamide at a temperature of about 80° C.

A compound of formula (XVIII-A) may be prepared by reducing and cyclizing the compound of formula (XVII-A).

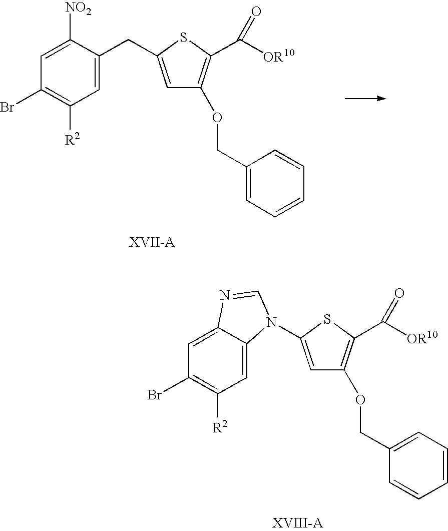

wherein all variables are as described above.

The step of reducing a compound of formula (XVII-A) may be carried out using conventional reduction techniques suitable for such compounds. Suitable reduction conditions will be apparent to those skilled in the art of organic synthesis and may include, for example, palladium on carbon under a hydrogen atmosphere, sulfided platinum on carbon under a hydrogen atmosphere, or iron powder in acetic acid. In one embodiment, the reduction may be effected using conditions such as sulfided platinum on carbon under a hydrogen atmosphere. The reaction may be carried out in an inert solvent at either atmospheric or elevated pressure. Suitable inert solvents include but are not limited to ethanol, methanol, and ethyl acetate.

Suitable cyclizing agents will be apparent to those skilled in the art of organic synthesis and include, for example triethylorthoformate or trimethylorthoformate, optionally in the presence of an acid catalyst, for example p-toluenesulfonic acid or pyridinium p-toluenesulfonate. In one embodiment, the cyclizing agent is triethylorthoformate and the catalyst is pyridinium p-toluenesulfonate. Conveniently, the reaction of a compound of formula (XVII-A) with the cyclization agent may be carried out neat, at a temperature of from about 25° C. to about 100° C. In one embodiment the reaction is carried out at about 25° C.

In another embodiment, the process of preparing a compound of formula (XVIII-A) may be conveniently carried out by performing a one-pot reduction-cyclization procedure on a compound of formula (XVII-A) using conditions such as sulfided platinum on carbon under a hydrogen atmosphere in the presence of triethylorthoformate and pyridinium p-toluenesulfonate. In this embodiment, triethylorthoformate may be used as a solvent or a co-solvent with another suitable inert solvent, such as ethyl acetate.

A compound of formula (XVII-A) may be prepared by reacting (e.g., coupling) a compound of formula (XVI) with 1,4-dibromo-2-nitrobenzene of formula (VIII).

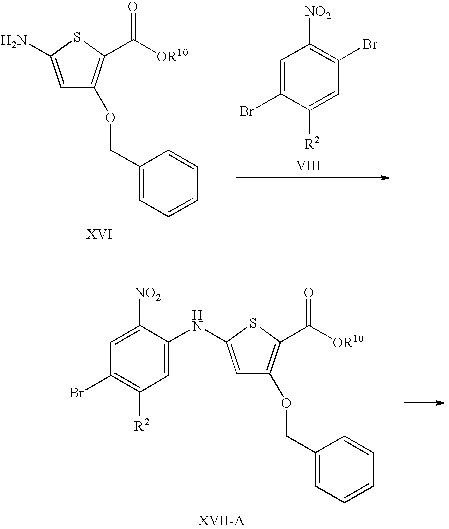

wherein all variables are as defined above.

The step of coupling a compound of formula (XVI) with 1,4-dibromo-2-nitrobenzene of formula (VII) to prepare a compound of formula (XVII-A) may be carried out using coupling techniques conventional in the art of organic synthesis. Examples of suitable coupling reactions include but are not limited to palladium-catalyzed cross-coupling conditions. Palladium catalyzed cross-coupling conditions include but are not limited to reacting the compound of formula (XVI) with 1,4-dibromo-2-nitrobenzene of formula (VIII) in the presence of a palladium source, optionally a phosphine ligand, and a base in a suitable inert solvent. Examples of suitable palladium sources include but are not limited to tris(dibenzylideneacetone)-dipalladium (0) or acetato(2′-di-t-butylphosphino-1,1′-biphenyl-2-yl)palladium (II). Examples of suitable phosphine ligands include but are not limited to 9,9-dimethyl-4,5-bis(diphenylphosphino)-xanthene. Examples of suitable bases include but are not limited to cesium carbonate, sodium methoxide, and triethylamine. Examples of suitable inert solvents include but are not limited to toluene or 1,4-dioxane. The reaction may be carried out at a temperature of between about room temperature and about 100° C. In one embodiment, the temperature is about 60° C. For a review of palladium-catalyzed cross-couplings of haloarenes and amines, see: Yang, B. H.; Buchwald, S. L. Journal of Organometallic Chemistry 1999, 576, 125-146. See also: Yin, J.; Zhao, M. M.; Huffman, M. A.; McNamara, J. M. Journal of Organic Chemistry 2002, 4, 3481-3484. 1,4-Dibromo-2-nitrobenzene compounds of formula (VIII) are commercially available or may be prepared using conventional techniques

A compound of formula (XVI) may be prepared by reducing a compound of formula (XV) using conventional reduction techniques.

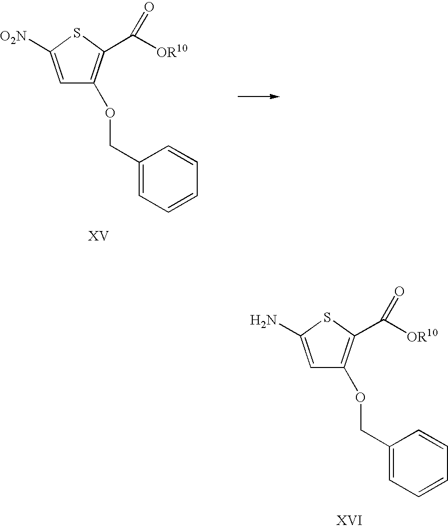

wherein all variables are as defined above.

Appropriate conditions for the reduction reaction will be apparent to those skilled in the art and include, for example, reducing agents, such as iron, in a suitable solvent, such as acetic acid. The reaction may be carried out with elevated temperatures, such as about 50° C.

A compounds of formula (XV) may be prepared by reacting a compound of formula (XIV) with benzyl bromide.

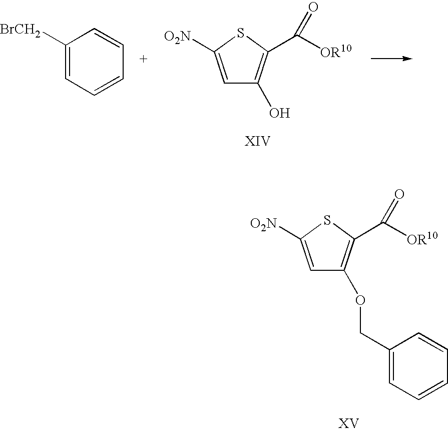

wherein all variables are as defined above.

This reaction may be carried out in an inert solvent, conveniently at room temperature, in the presence of a suitable base. The compound of formula (XIV) and benzyl bromide may be present in equimolar amounts; however, a slight excess of benzyl bromide may be employed if desired. Examples of suitable bases for this reaction include but are not limited to, potassium carbonate, sodium carbonate, cesium carbonate, sodium hydride, and potassium hydride. Examples of suitable inert solvents for this reaction include but are not limited to, N,N-dimethylformamide, tetrahydrofuran, dioxane, and 1,2-dimethoxyethane.

As shown below, the order of the steps in the foregoing reaction is not critical to the process and the steps may be carried out in any suitable order as determined by those skilled in the art. For example, in another embodiment of the present invention, the compounds of formula (V) may be prepared by the process out-lined in Scheme 3.

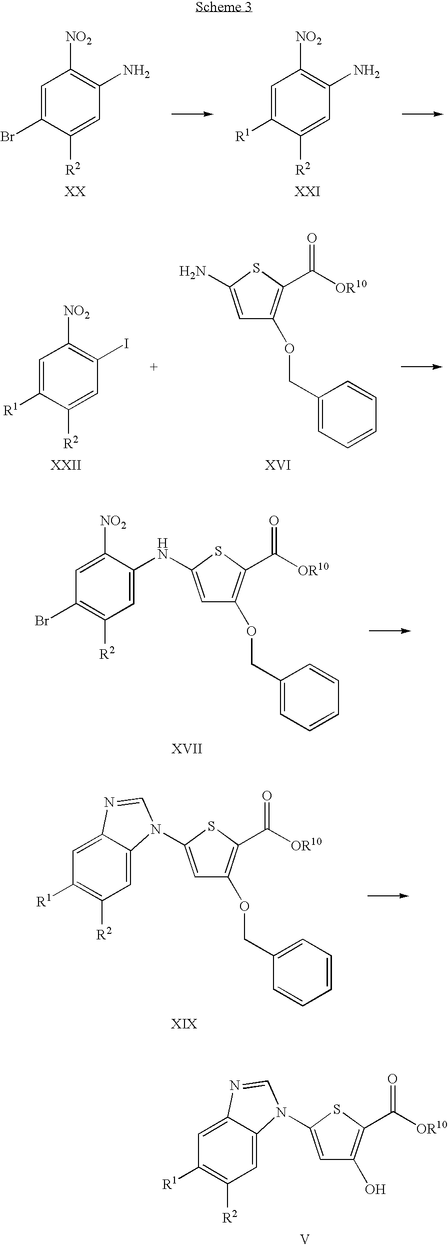

wherein:
R¹⁰ is selected from alkyl and suitable carboxylic acid protecting groups;
Y¹ is —O—; and
   all other variables are as defined above.

In particular, this process for preparing the compounds of formula (V) (all formulas and all variables having been defined above) comprises the steps of:
a) reacting 4-bromo-2-nitroaniline of formula (XX) using a conventional cross-coupling reaction to prepare a compound of formula (XXI);
b) reacting the compound of formula (XXI) with iodine and t-butyl nitrite to prepare a compound of formula (XXII);
c) reacting the compound of formula (XXII) with a compound of formula (XVI) to prepare a compound of formula (XVII);
d) reducing and cyclizing the compound of formula (XVII) to prepare a compound of formula (XIX);
e) reacting the compound of formula (XIX) with acid to prepare a compound of formula (V).

The reaction of the compound of formula (XIX) with acid to prepare a compound of formula (V) is described above.

According to this process, a compound of formula (XIX) may be prepared by reducing and cyclizing the compound of formula (XVII) using conditions analogous to those described above for the preparation of a compound of formula (XIX) from a compound of formula (XVIII).

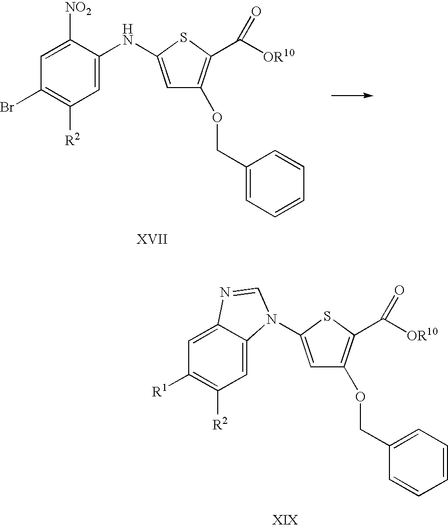

wherein all variables are as defined above.

A compound of formula (XVII) may be prepared by reacting a compound of formula (XXII) with a compound of formula (XVI) using conditions described above for the reaction of a compound of formula (XVI) with 1,4-dibromo-2-nitrobenzene of formula (VIII).

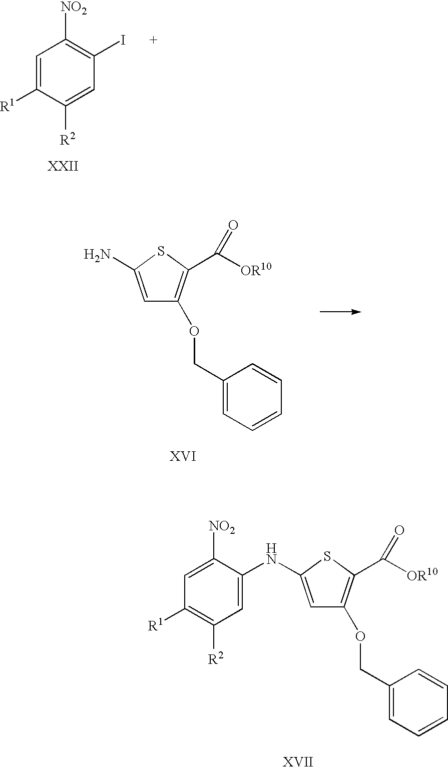

wherein all variables are as defined above.

A compound of formula (XXII) may be prepared by reacting a compound of formula (XXI) with iodine and t-butyl nitrite.

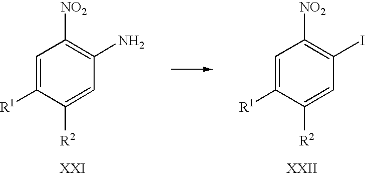

wherein all variables are as defined above.

This reaction may be carried out using a Sandmeyer-like reaction known to those skilled in the art. For transformation of aryl amines to aryl halides, see: Larock, R. Comprehensive Organic Transformation (2nd Edition), John Wiley & Sons, Inc. (1999) 678-679. The compound of formula (XXII) may be prepared by reacting a compound of formula (XXI) in an inert atmosphere, at a temperature of 60° C., with iodine and tert-butyl nitrite, in a suitable solvent, such as acetonitrile.

Compounds of formula (XXI) may be prepared by reacting 4-bromo-2-nitroaniline of formula (XX) using conventional cross-coupling reactions such as those described above.

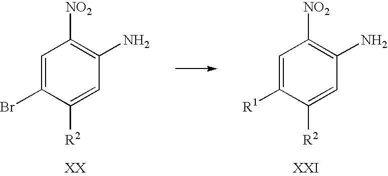

wherein all variables are as defined above.

4-Bromo-2-nitroaniline compounds of formula (XX) are commercially available or may be prepared using conventional techniques.

In one particular embodiment, the compounds of the invention may be conveniently prepared by the methods outlined in Scheme 4 below.

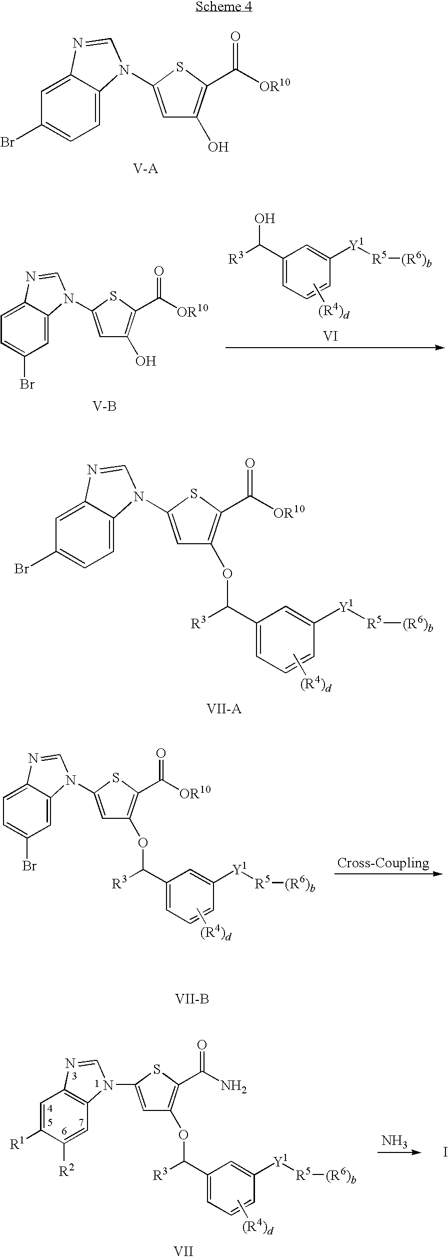

wherein:
Y¹ is —O—;
R¹⁰ is selected alkyl and suitable carboxylic acid protecting groups; and all other variables are as defined above.

Generally, the process for preparing compounds of the invention (all formulas and all variables having been defined above) comprises the steps of:
a) reacting regioisomer compounds of formula (V-A) and (V-B) with a compound of formula (VI) to prepare regioisomer compounds of formula (VII-A) and (VII-B);
b) reacting the regioisomer compounds of formula (VII-A) and (VII-B) under conventional cross-coupling reaction conditions to prepare a compound of formula (VII);
c) optionally separating the compound of formula (I) into enantiomers;
d) optionally converting the compound of formula (I) to a pharmaceutically acceptable salt or solvate thereof; and
e) optionally converting the compound of formula (I) or a pharmaceutically acceptable salt or solvate thereof to a different compound of formula (I) or a pharmaceutically acceptable salt or solvate thereof.

As will be apparent to those skilled in the art, the order of the steps in the foregoing reaction is not critical to the practice of the process of the present invention. The foregoing reaction steps may be carried out in any suitable order based upon the knowledge of those skilled in the art. Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art.

The reaction of the compound of formula (VII) with ammonia to prepare a compound of formula (I) and the separation of a compound of formula (I) into enantiomers and the formation of pharmaceutically acceptable salts and solvates thereof are all described above.

Compounds of formula (VII-A) and (VII-B) may be prepared by reacting the compound of formula (V-A) or the compound of formula (V-B), respectively, with a compound of formula (VI) under Mitsunobu reaction conditions, as described above. Br in the compounds of formula (VII-A) and (VII-B) may be further converted to other functional groups using chemistry transformation known to those skilled in the art, for example, conventional cross-coupling reactions to prepare a different compound of formula (VII).

More particularly, the compounds of formula (VII) may be prepared from compounds of formula (VII-A and VII-B) using palladium-catalyzed Suzuki, Stille, or Negishi cross-coupling techniques (described above) which are conventional in the art of organic synthesis.

As will be apparent to those skilled in the art, the order of the steps in the foregoing reaction is not critical to the practice of the process of the present invention. For example, the compounds of formula (VII) may also be prepared by altering the order of the steps such that the cross-coupling reaction is carried out on the regioisomer compounds of formula (V-A) and (V-B) to prepare a compound of formula (V) (as defined in Scheme 1 above) followed by the reaction of a compound of formula (V) with a compound of formula (VI) to prepare a compound of formula (VII). Each of these reaction steps may be carried out using the techniques described above.

As a further embodiment, the compounds of formula (VII-A) and (VII-B) may first be reacted with ammonia to produce the corresponding Br-substituted compounds of formula (I), followed by the cross-coupling reaction to prepare a different compound of formula (I) wherein the Br substituent is displaced by another functional group defined by R¹ and R² above.

The compounds of formula (V-A) and (V-B) are prepared by reacting 5-bromobenzimidazole with a compound of formula (III).

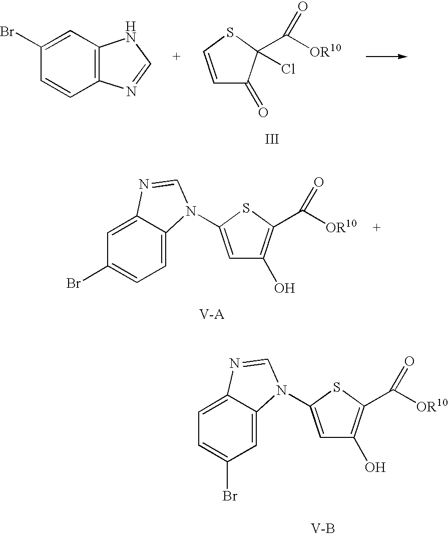

wherein all variables are as defined above.

This reaction may be carried out using the same reaction conditions described above for the preparation of a compound of formula (V).

In another embodiment, the present invention provides another process for preparing compounds of the invention, which is outlined in Scheme 5 below.

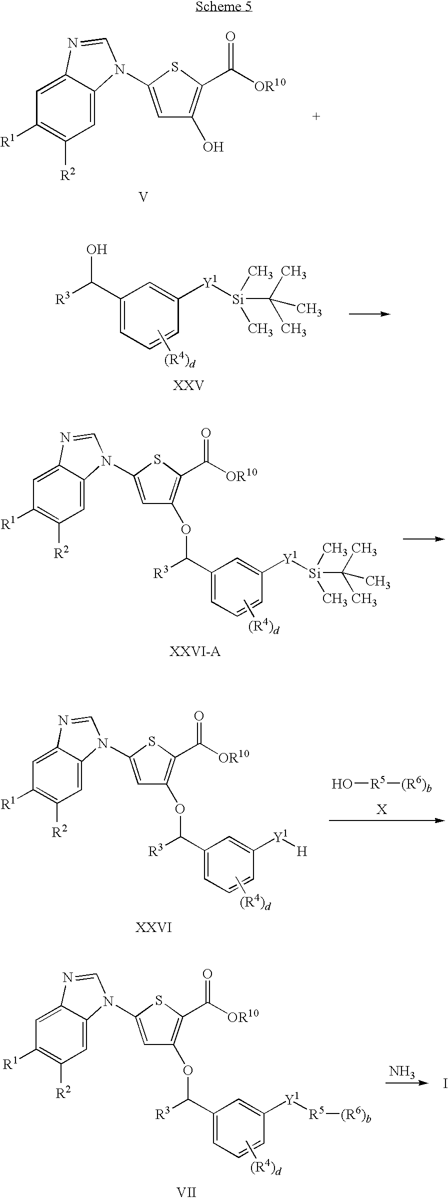

wherein:
R¹⁰ is selected from alkyl and suitable carboxylic acid protecting groups;
Y¹ is —O—; and
   all other variables are as defined above.

Generally, the process for preparing the compounds of the invention (all formulas and all variables having been defined above) comprises the steps of:
a) reacting the compound of formula (V) with a compound of formula (XXV) to prepare a compound of formula (XXVI-A) and removing the protecting group to prepare a compound of formula (XXVI);
b) reacting the compound of formula (XXVI) with a compound of formula (X) to prepare a compound of formula (VII);
c) reacting the compound of formula (VII) with ammonia to prepare a compound of formula (I);
d) optionally separating the compound of formula (I) into enantiomers;
e) optionally converting the compound of formula (I) to a pharmaceutically acceptable salt or solvate thereof; and
f) optionally converting the compound of formula (I) or a pharmaceutically acceptable salt or solvate thereof to a different compound of formula (I) or a pharmaceutically acceptable salt or solvate thereof.

As will be apparent to those skilled in the art, the order of the steps in the foregoing reaction is not critical to the practice of the process of the present invention. The foregoing reaction steps may be carried out in any suitable order based upon the knowledge of those skilled in the art. Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art.

The reaction of the compound of formula (VII) with ammonia to prepare a compound of formula (I) and the separation of a compound of formula (I) into enantiomers and the formation of pharmaceutically acceptable salts and solvates thereof are all described above.

According to this method, a compound of formula (VII) is prepared by reacting the compound of formula (XXVI) with a compound of formula (X) using conventional Mitsunobu reaction conditions such as those described above for preparation of the compound of formula (VII) by reaction of the compound of formula (V) with a compound of formula (VI).

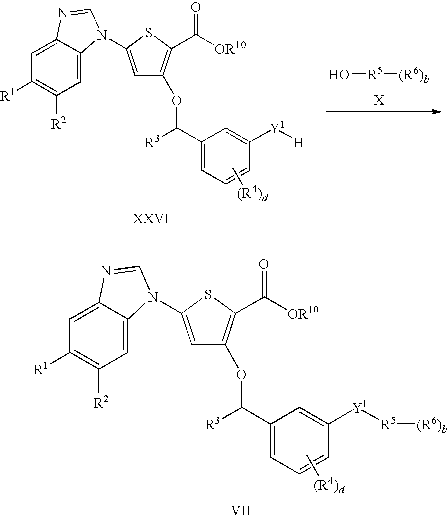

wherein all variables are as defined above.

If desired, the enantiomers of the compound of formula (VII) may be separated as described above to yield the enantiomerically enriched compounds of formula (VII-1) and (VII-2), which may then be used in the foregoing process to ultimately yield an enantiomerically enriched compound of formula (I-1) or (I-2), respectively.

Compounds of formula (X) are commercially available or may be prepared using conventional techniques. A compound of formula (XXVI) may be prepared by removing the silyl protecting group from the compound of formula (XXVI-A) using conventional techniques, such as reaction with tetrabutylammonium fluoride. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

A compound of formula (XXVI-A) may be prepared by reacting a compound of formula (V) with a compound of formula (XXV) using conventional Mitsunobu reaction conditions such as those described.

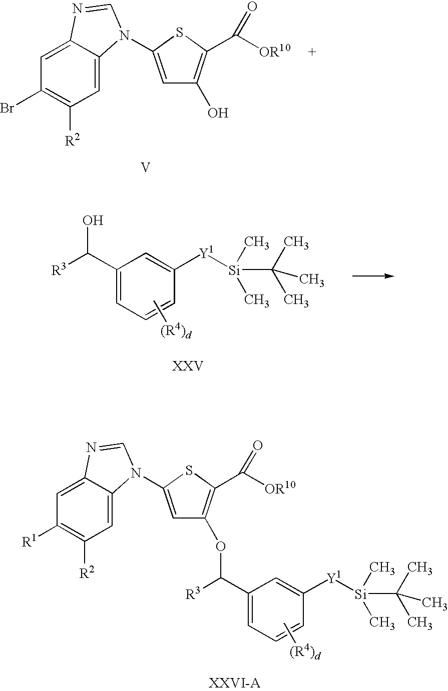

wherein all variables are as defined above.

If desired, the enantiomers of the compound of formula (XXVI-A) may be separated using techniques described above to yield the enantiomerically enriched compounds of formula (XXVI-A1) and (XXVI-A2),

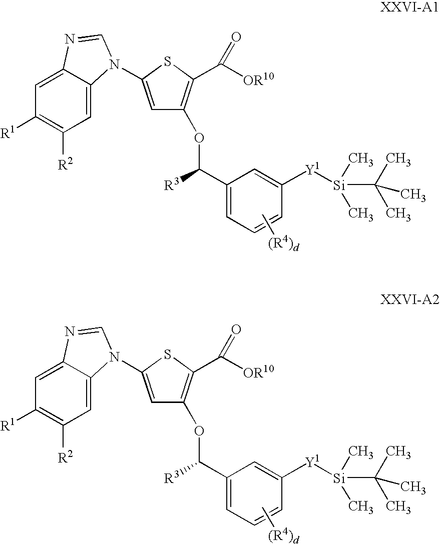

which may then be used in the foregoing process to ultimately yield an enantiomerically enriched compound of formula (I-1) or (I-2), respectively.

Processes for the preparation of compounds of formula (V) are described above.

Compounds of formula (XXV) may be prepared according to the following reaction scheme.

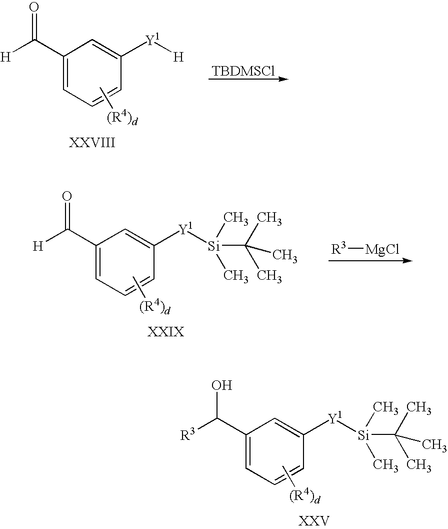

wherein all variables are as defined above.

The compounds of formula (XXVIII) are commercially available or may be prepared using conventional techniques known to those skilled in the art. The t-butyl-dimethylsilyl protecting group is installed using conventional techniques to prepare the compound of formula (XXIX). See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991. The compound of formula (XXIX) is reacted with a magnesium chloride of the formula R³—MgCl to prepare the compound of formula (XXV). If desired, the enantiomers of the compound of formula (XXV) may be separated using conventional separation techniques (e.g., supercritical fluid chromatography (SFC)) to yield the enantiomerically enriched compound of formula (XXV-1)

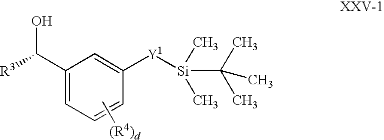

which may then be used in the foregoing process to ultimately yield an enantiomerically enriched compound of formula (I-1).

In another embodiment, the present invention provides another process for preparing compounds of the invention, which is out-lined in Scheme 6 below.

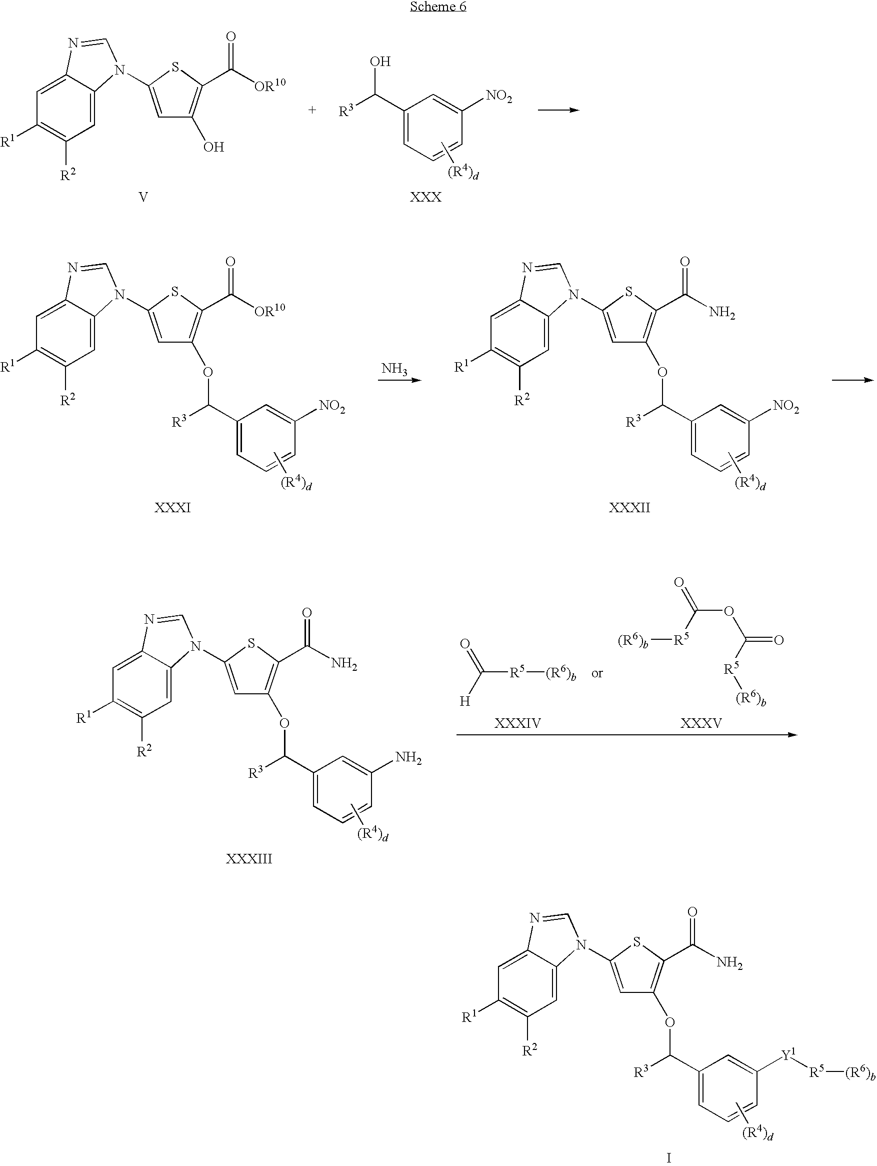

wherein:
R¹⁰ is selected from alkyl and suitable carboxylic acid protecting groups;
Y¹ is —NR⁷— or —N(H)C(O)—; and
   all other variables are as defined above.

Generally, the process for preparing the compounds of the invention (all formulas and all variables having been defined above) comprises the steps of:
a) reacting the compound of formula (V) with a compound of formula (XXX) to prepare a compound of formula (XXXI);
b) reacting the compound of formula (XXXI) with ammonia to prepare a compound of formula (XXXII);
c) reducing the compound of formula (XXXII) to prepare a compound of formula (XXXIII);
d) reacting the compound of formula (XXXIII) with a compound of formula (XXXIV) or (XXXV) to prepare a compound of formula (I);
e) optionally separating the compound of formula (I) into enantiomers;
f) optionally converting the compound of formula (I) to a pharmaceutically acceptable salt or solvate thereof; and
g) optionally converting the compound of formula (I) or a pharmaceutically acceptable salt or solvate thereof to a different compound of formula (I) or a pharmaceutically acceptable salt or solvate thereof.

As will be apparent to those skilled in the art, the order of the steps in the foregoing reaction is not critical to the practice of the process of the present invention. The foregoing reaction steps may be carried out in any suitable order based upon the knowledge of those skilled in the art. Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art.

More specifically, according to this method, a compound of formula (I) wherein Y¹ is —NR⁷— may be prepared by reacting the compound of formula (XXXIII) with a compound of formula (XXXIV) using conventional reductive amination reaction conditions. See, Larock, R. C. Comprehensive Organic Transformation (2^{nd} Edition), Wiley-VCH, 1999. Similarly, amide bond forming conditions may be employed to prepare a compound of formula (I) wherein Y¹ is —N(H)C(O)— by reacting the compound of formula (XXXIII) with a compound of formula (XXXV).

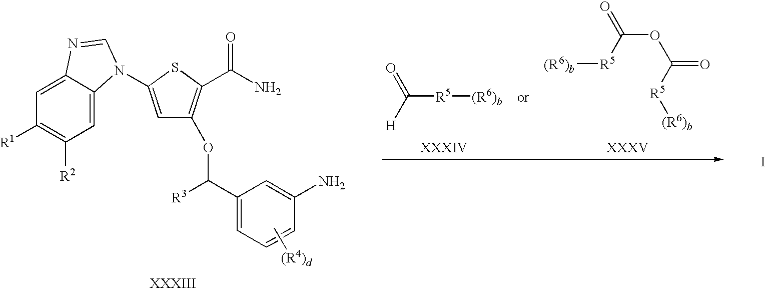

wherein all variables are as defined above.

Compounds of formula (XXXIII) may be prepared by reduction of a compound of formula (XXXII) using conventional nitro reaction conditions such as those described above.

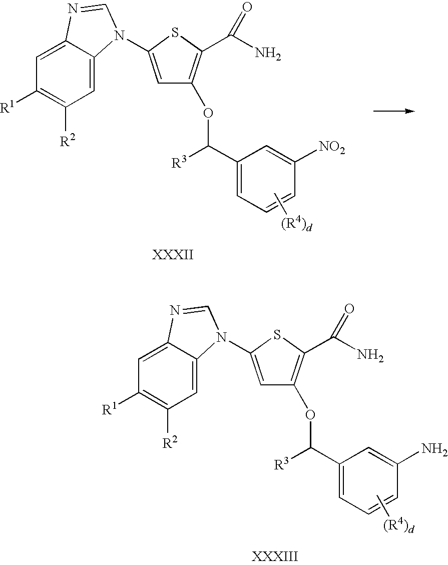

wherein all variables are as defined above.

If desired, the enantiomers of the compound of formula (XXXIII) may be separated using conventional separation techniques (e.g., SFC) to yield the enantiomerically enriched compounds of formula (XXXIII-1) and (XXXIII-2)

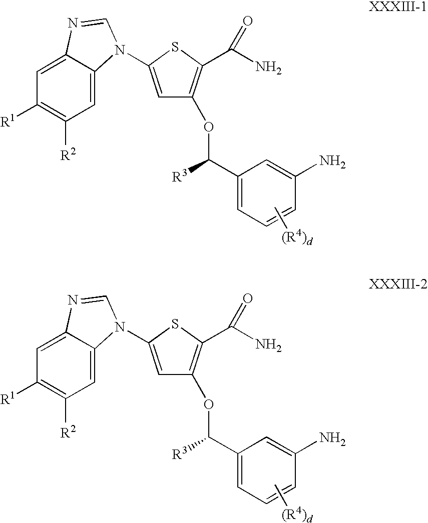

which may then be used in the foregoing process to ultimately yield an enantiomerically enriched compound of formula (I-1) or (I-2), respectively.

Compounds of formula (XXXII) may be prepared by reaction of the compound of formula (XXXI) with ammonia using reaction conditions such as those described above.

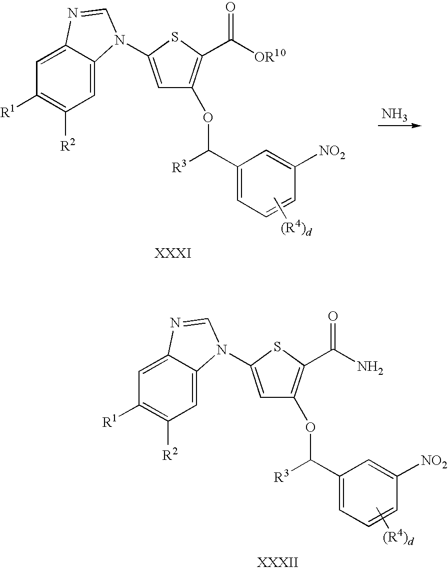

wherein all variables are as defined above.

If desired, the enantiomers of the compound of formula (XXXII) may be separated using conventional separation techniques (e.g., SFC) to yield the enantiomerically enriched compounds of formula (XXXII-1) and (XXXII-2)

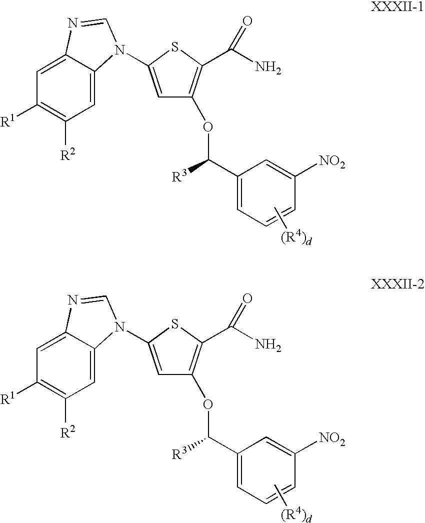

which may then be used in the foregoing process to ultimately yield an enantiomerically enriched compound of formula (I-1) or (I-2), respectively.

Compounds of formula (XXXI) may be prepared by reacting a compound of formula (V) with a compound of formula (XXX) using conventional Mitsunobu reaction conditions such as those described above.

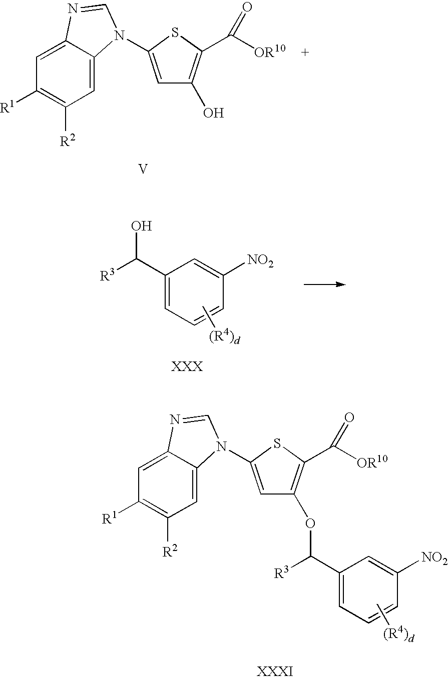

wherein all variables are as defined above.

If desired, the enantiomers of the compound of formula (XXXI) may be separated using conventional separation techniques (e.g., SFC) to yield the enantiomerically enriched compounds of formula (XXXI-1) and (XXXI-2)

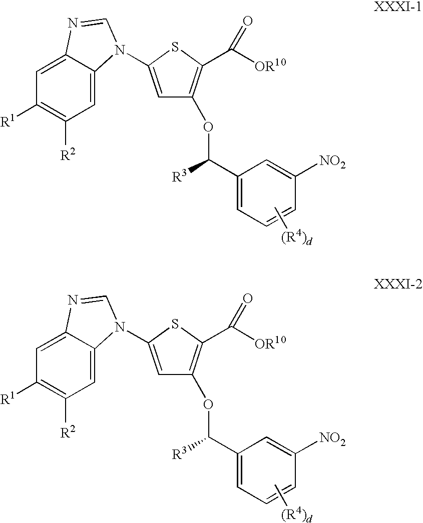

which may then be used in the foregoing process to ultimately yield an enantiomerically enriched compound of formula (I-1) or (I-2), respectively. Compounds of formula (XXX) may be prepared as follows.

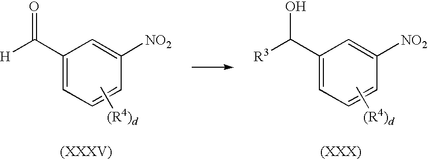

wherein all variables are as defined above.

The compounds of formula (XXXV) are commercially available or may be prepared using conventional techniques known to those skilled in the art.

The compound of formula (XXXV) is reacted with a magnesium chloride of the formula R³—MgCl to prepare the compound of formula (XXX). If desired, the enantiomers of the compound of formula (XXX) may be separated using conventional separation techniques (e.g., supercritical fluid chromatography (SFC)) to yield the enantiomerically enriched compound of formula (XXX-1)

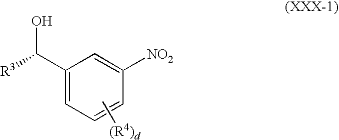

which may then be used in the foregoing process to ultimately yield an enantiomerically enriched compound of formula (I-1).

In another embodiment, the present invention provides another process for preparing compounds of the invention, which is out-lined in Scheme 7 below.

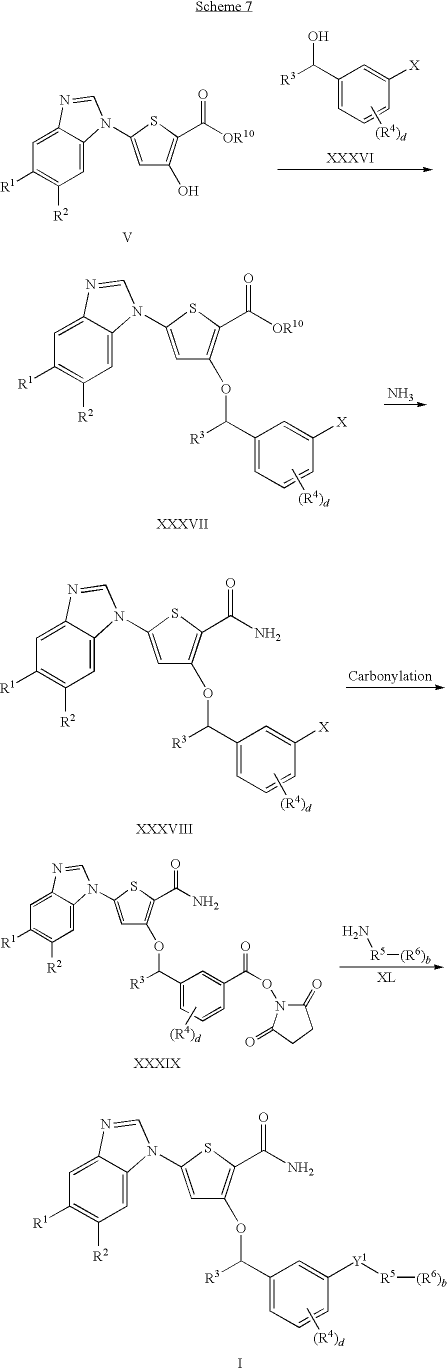

wherein:
X is Br or I
Y¹ is —C(O)N(H)—;
   and other variables are as defined above.

Generally, the process for preparing the compounds of the invention (all formulas and all variables having been defined above) comprises the steps of:
a) reacting the compound of formula (V) with a compound of formula (XXXVI) to prepare a compound of formula (XXXVII);
b) reacting the compound of formula (XXXVII) with ammonia to prepare a compound of formula (XXXVIII);
c) reacting the compound of formula (XXXVIII) with carbon monoxide and N-hydroxysuccinimide in the presence of a catalyst to prepare a compound of formula (XXXIX);
d) reacting the compound of formula (XXXIX) with an amine of formula (XL) to prepare a compound of formula (I);
e) optionally separating the compound of formula (I) into enantiomers;
f) optionally converting the compound of formula (I) to a pharmaceutically acceptable salt or solvate thereof; and
g) optionally converting the compound of formula (I) or a pharmaceutically acceptable salt or solvate thereof to a different compound of formula (I) or a pharmaceutically acceptable salt or solvate thereof.

As will be apparent to those skilled in the art, the order of the steps in the foregoing reaction is not critical to the practice of the process of the present invention. The foregoing reaction steps may be carried out in any suitable order based upon the knowledge of those skilled in the art. Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art.

More specifically, according to this method, a compound of formula (I) wherein Y¹ is —C(O)N(H)—, may be prepared by reacting the compound of formula (XXXIX) with an amine of formula (XL) in an inert solvent.

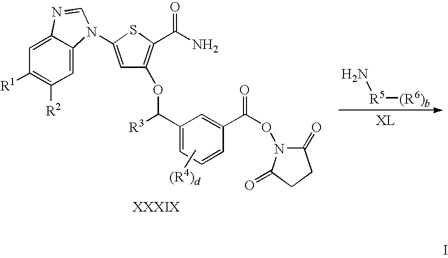

wherein all variables are as defined above.

Compounds of formula (XXXIX) may be prepared by reaction of the compound of formula (XXXVIII) with carbon monoxide and N-hydroxysuccinimide in the presence of a suitable catalyst.

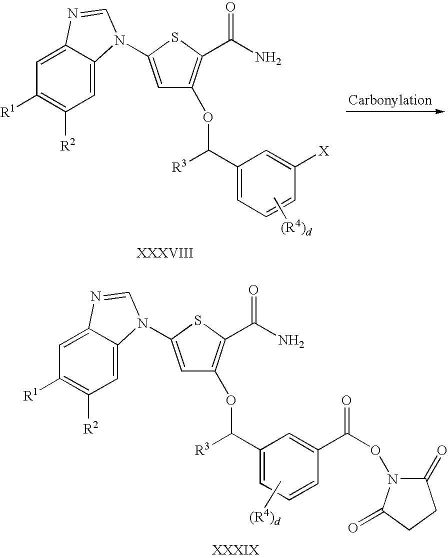

wherein all variables are as defined above.

Compounds of formula (XXXVIII) may be prepared by reaction of the compound of formula (XXXVII) with ammonia using reaction conditions such as those described above for the reaction of a compound of formula (XXXI) with ammonia.

If desired, the enantiomers of the compound of formula (XXXVIII) may be separated using conventional separation techniques (e.g., SFC) to yield the enantiomerically enriched compounds of formula (XXXVIII-1) and (XXXVIII-2)

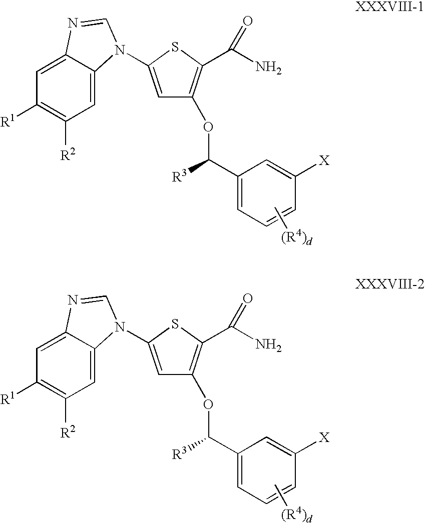

which may then be used in the process to ultimately yield an enantiomerically enriched compound of formula (I-1) or (I-2), respectively.

Compounds of formula (XXXVII) may be prepared by reacting a compound of formula (V) with a compound of formula (XXXVI) using conventional Mitsunobu reaction conditions such as those described above for the reaction of a compound of formula (V) with a compound of formula (XXX).

If desired, the enantiomers of the compound of formula (XXXVII) may be separated using conventional separation techniques (e.g., SFC) to yield the enantiomerically enriched compounds of formula (XXXVII-1) and (XXXVII-2)

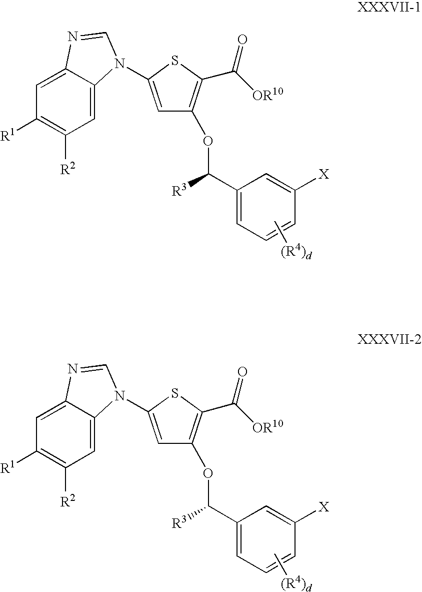

which may then be used in the process to ultimately yield an enantiomerically enriched compound of formula (I-1) or (I-2), respectively.

Compounds of formula (XXXVI) may be prepared from commercially available starting materials using conventional techniques, in a manner analogous to that described for the preparation of compounds of formula (XXX).

If desired, the enantiomers of the compound of formula (XXX) may be separated using conventional separation techniques (e.g., supercritical fluid chromatography (SFC)) to yield the enantiomerically enriched compound which may be used in the process to ultimately yield an enantiomerically enriched compound of formula (I-1).

A compound of formula (I) maybe converted into a different compound of formula (I) using techniques known to those skilled in the art.

In one embodiment, a compound of formula (I-1A) may be converted to a compound of formula (I-1B) using oxidation conditions. A compound of formula (I-1B) may be converted to a compound of formula (I-1C) using standard deprotection conditions.

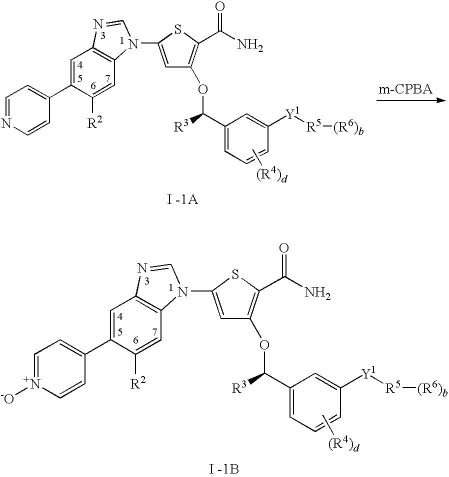

wherein all variables are as defined above.

A compound of formula (I-1A) may be converted to a compound of formula (I-1B) using oxidizing agents such as m-chloroperoxybenzoic acid (m-CPBA) in appropriate solvents such as dichloromethane or chloroform at room temperature.

Based upon this disclosure and the examples contained herein one skilled in the art can readily convert a compound of formula (I) or (I-1) or a pharmaceutically acceptable salt or solvate thereof into another compound of formula (I) or (I-1) or a pharmaceutically acceptable salt or solvate thereof. The following abbreviations as employed in the examples, have the recited meanings.

The following abbreviations as employed in the examples, have the recited meanings.
g gram(s)
mg milligram(s)
mol mole(s)
mmol millimole(s)
N normal
L liter(s)
mL milliliter(s)
μL microliter(s)
h hour(s)
min minute(s)
° C. degrees Centigrade
HCl hydrochloric acid
DCM dichloromethane
CHCl₃ chloroform
MeOH methanol
EtOH ethanol
i-PrOH isopropanol
EtOAc ethyl acetate
THF tetrahydrofuran
TFA trifluoroacetic acid
DMA N,N-dimethylacetamide
DMF N,N-dimethylformamide
NH₄Cl ammonium chloride
MgSO₄ magnesium sulfate
NaOH sodium hydroxide
NaHCO₃ sodium bicarbonate
Na₂CO₃ sodium carbonate
K₂CO₃ potassium carbonate
Cs₂CO₃ cesium carbonate
Na₂SO₄ sodium sulfate
N₂ nitrogen
H₂ hydrogen
rt room temperature
Cl₂Pd(dppf) dichloro[1,1′-bis(diphenylphosphino)ferrocene] palladium(II)
XANTPHOS (4,5-Bis(diphenylphosphino)-9,9-dimethylxanthene) is a commercially available catalyst, from Aldrich
SFC supercritical fluid chromatography
TLC thin layer chromatography.
ee enantiomeric excess

Reagents are commercially available or are prepared according to procedures in the literature. In the following structures, “Me” refers to the group —CH₃.

All references to “ether” are to diethyl ether; brine refers to a saturated aqueous solution of NaCl. Unless otherwise indicated, all temperatures are expressed in ° C. (degrees Centigrade). All reactions are conducted under an inert atmosphere at rt unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Isco, Inc. Sq 16× or 100 sg Combiflash).

Reported HPLC retention times (RT) were obtained on a Waters 2795 instrument attached to a Waters 996 diode array detector reading 210-500 nm. The column used was a Synergi Max-R^{P} (50×2 mm) model #00B-4337-B0. Solvent gradient was 15% MeOH:water to 100% MeOH (0.1% formic acid) over 6 min. Flow rate was 0.8 mL/min. Injection volume was 3 μL.

### Intermediate 1: (1S)-1-(2-Chloro-3-nitrophenyl)ethanol

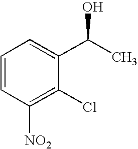

To ether cooled to −78° C. was added titanium (IV) chloride (0.85 mL, 7.8 mmol) and a 1.6M solution of methyl lithium in ether (4.9 mL, 7.8 mmol). After warming the mixture to −40° C., it was transferred via double-tipped needle to a −78° C. ether solution of 2-chloro-3-nitrobenzaldehyde (1.04 g, 5.6 mmol), which can be synthesized according to the procedure in J. Med. Chem. 1988, 31, 936-944 The reaction was allowed to slowly warm to rt and was quenched with the addition of MeOH and water. The layers were separated, and the aqueous phase was extracted with EtOAc. The combined organic phases were washed with brine, dried over MgSO₄ and concentrated to an oil. The crude material was purified by flash column chromatography (10% EtOAc:hexanes) to give 0.96 g of the racemic compound (84%). The enantiomers were separated using packed column supercritical fluid chromatography (SFC) on a 3×25 cm Daicel® AD-H column with a 90 g/min total flow (81 g/min CO₂-90%) (9 g/min MeOH-10%) to give the title compound as a yellow oil. ¹H NMR (400 MHz, d₆-DMSO) δ 7.86 (m, 1H), 7.58 (m, 1H), 5.62 (d, J=4.4 Hz, 1H), 5.06 (m, 1H), 1.30 (d, J=6.4 Hz, 3H).

### Intermediate 2: (1S)-1-(2-chloro-3-{[(1,1-dimethylethyl)(dimethyl)silyl]oxy}phenyl)ethanol

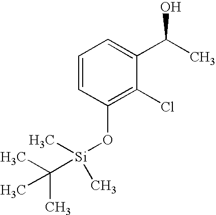

Step A—1-(2-Chloro-3-{[(1,1-dimethylethyl)(dimethyl)silyl]oxy}phenyl)ethanone

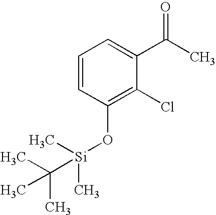

To a solution of 1-(2-chloro-3-hydroxyphenyl)ethanone (8.4 g, 50 mmol) which may be synthesized according to the procedure in Proceedings of the Indiana Academy of Science 1983, 92, 145-151 and imidazole (3.8 g, 55 mmol) in DCM (100 mL) was added chloro(tert-butyl)dimethylsilane (8.3 g, 55 mmol). The solution was stirred for 1 h and silica (20 g) was added. The volatiles were evaporated under reduced pressure, and the pre-adsorbed solids were loaded into a solid loading cartridge and subjected to a gradient elution using hexanes (100%) to hexanes:EtOAc (90:10) using a RediSep silica gel cartridge (120 g; ISCO). The appropriate fractions were combined and concentrated under reduced pressure to give 7.1 g (25 mmol) of the title compound. ¹H NMR (400 MHz, CDCl₃): δ 7.16 (dd, J=8.0, 7.7 Hz, 1H), 7.04 (dd, J=7.7, 1.5 Hz, 1H), 6.96 (dd, J=8.0, 1.5 Hz, 1H), 2.60 (s, 3H), 1.02 (s, 9H), 0.23 (s, 6H).

### Step B—(1S)-1-(2-chloro-3-{[(1,1-dimethylethyl)(dimethyl)silyl]oxy}phenyl)ethanol (title compound)

To a solution of borane, dimethylsulfide complex (1.8 mL, 30 mmol) in THF (10 mL) was added a 1M solution of (R)-1-methyl-3,3-diphenyltetrahydro-3H-pyrrolo[1,2-c][1,3,2]oxazaborole in toluene (0.25 mL, 0.25 mmol). To this mixture was slowly added over 2 h a solution of 1-(2-chloro-3-{[(1,1-dimethylethyl)(dimethyl)silyl]oxy}phenyl)ethanone (7.1 g, 25 mmol) in THF (50 mL). The solution was stirred an additional 18 h then MeOH was added dropwise to quench any excess borane. The volatiles were evaporated under reduced pressure, and DCM was added (50 mL). The resulting white solid was removed by filtration and the silica was added to the filtrate. The volatiles were evaporated under reduced pressure and the pre-adsorbed solids were loaded into a solid loading cartridge and subjected to a gradient elution using hexanes (100%) to hexanes:EtOAc (80:20) using a RediSep silica gel cartridge (120 g; ISCO). The appropriate fractions were combined and concentrated under reduced pressure to give 6.8 g (24 mmol) of the title compound as a white solid. ¹H NMR (400 MHz, CDCl₃): δ 7.19-7.12 (m, 2H), 6.81-6.79 (m, 1H), 5.30-5.25 (m, 1H), 1.93 (d, J=3.6 Hz, 1H) 1.47 (d, J=6.4 Hz, 3H), 1.02 (s, 9H), 0.21 (s, 3H), 0.21 (s, 3H).

Alternatively, Intermediate 2 can be prepare by the following method.

### Step A—2-chloro-3-{[(1,1-dimethylethyl)(dimethyl)silyl]oxy}benzaldehyde

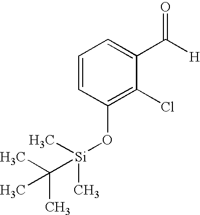

To a solution of 2-chloro-3-hydroxybenzaldehyde (30.0 g, 192 mmol) which was purchased from Sigma-Aldrich and imidazole (15.6 g, 230 mmol) in THF (200 mL) was added chloro(tert-butyl)dimethylsilane (30.0 g, 200 mmol). The solution was stirred for overnight. The solution was poured into water and extracted with ether (2×300 mL). The ether layers were dried (MgSO₄), filtered and the volatiles removed under reduced pressure to give 51.0 g (188 mmol) of the title compound. ¹H NMR (400 MHz, CDCl₃): δ 10.49 (s, 1H), 7.54 (dd, J=7.7, 1.6 Hz, 1H), 7.24 (dd, J=8.0, 7.7 Hz, 1H), 7.13 (dd, J=8.0, 1.6 Hz, 1H), 1.05 (s, 9H), 0.25 (s, 6H).

### Step B—(1S)-1-(2-chloro-3-{[(1,1-dimethylethyl)(dimethyl)silyl]oxy}phenyl)ethanol (title compound)

To a solution of 2-chloro-3-{[(1,1-dimethylethyl)(dimethyl)silyl]oxy}-benzaldehyde (50.0 g, 184 mmol) in THF (500 mL) cooled to −78° C. was added a 3M solution of methylmagnesiumchloride in THF (67.0 mL, 202 mmol). The solution was allowed to warm to rt and then water was added to quenched the reaction. The solution was extracted with ether, dried (MgSO₄), filtered and the volatiles were evaporated under reduced pressure to give 50.0 g of the racemic title compound as a colorless oil. The enantiomers were separated using SFC on a 3×25 cm OJ-H column with a 90 g/min total flow, 92/8 CO₂/MeOH, 103 bar, 27° C. The desired (S) enantiomer eluted first under these separation conditions. Upon standing, the enantiopure title compound solidified.

### Intermediate 3: Methyl 5-(5-bromo-1H-benzimidazol-1-yl)-3-{[tert-butyl(dimethyl)silyl]oxy}thiophene-2-carboxylate; and

### Intermediate 4: Methyl 5-(6-bromo-1H-benzimidazol-1-yl)-3-{[tert-butyl(dimethyl)silyl]oxy}thiophene-2-carboxylate

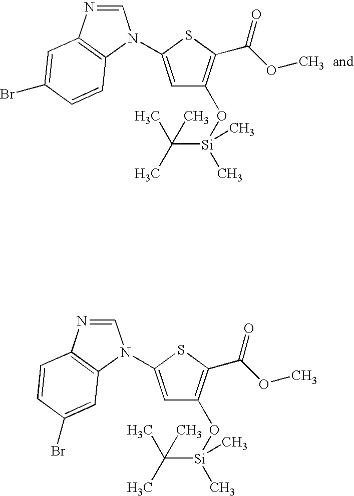

### Step A—4-Bromobenzene-1,2-diamine

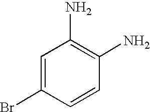

A mixture of 4-bromo-2-nitroaniline (50 g, 230 mmol) and tin (II) chloride (174 g, 920 mmol) in 1.2 L of EtOH was heated at 80° C. for 16 h. The reaction was cooled to rt and brought to a basic pH with the addition of 5N and 1N NaOH. Once basic, 2 L of EtOAc was added and the mixture stirred. The organic layer was decanted off. This process was repeated until the EtOAc decant provided very little material. The organic solution was washed with brine, dried over MgSO₄ and concentrated to give 48.9 g of crude product. ¹H NMR (400 MHz, d₆-DMSO) δ 6.60 (d, J=2.4 Hz, 1H), 6.45 (dd, J=8.0 and 2.4 Hz, 1H), 6.39 (d, J=8.0 Hz, 1H), 4.63 (brs, 4H).

### Step B—5-Bromo-1H-benzimidazole

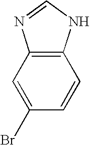

A solution of crude, impure 4-bromobenzene-1,2-diamine (48.87 g, 230 mmol), trimethylorthoformate (75 mL, 690 mmol), and 6 mL of formic acid was heated at 80° C. After 16 h, the reaction was concentrated to give 46.2 g of a crude, impure orange residue. ¹H NMR (400 MHz, d₆-DMSO) δ 8.24 (s, 1H), 7.77 (d, J=1.6 Hz, 1H), 7.53 (d, J=8.8 Hz, 1H), 7.30 (dd, J=8.6 and 1.8 Hz, 1H).

### Step C—Methyl 5-(5-bromo-1H-benzimidazol-1-yl)-3-{[tert-butyl(dimethyl)silyl]oxy}thiophene-2-carboxylate and methyl 5-(6-bromo-1H-benzimidazol-1-yl)-3-{[tert-butyl(dimethyl)silyl]oxy}thiophene-2-carboxylate (title compounds)

To a solution of crude, impure 5-bromobenzimidazole (46.2 g) and methyl 2-chloro-3-oxo-2,3-dihydrothiophene-2-carboxylate (Synthesis, 1984, 10, 847-850) (42 g, 220 mmol) in 800 mL of CHCl₃ was added N-methylimidazole (28 mL, 345 mmol). After 16 h, N-methylimidazole (17 mL, 220 mmol) and tert-butylchlorodimethylsilane (36 g, 240 mmol) was added. When TLC showed the reaction to be complete, the solution was diluted with water. The layers were separated. The organic phase was washed with water, dried over MgSO₄ and concentrated onto celite. The crude mixture was purified by flash column chromatography (0-25% EtOAc:hexanes) in batches to separate the 2 regioisomers, giving 33.5 g of Intermediate 3 eluting first and 29.2 g of Intermediate 4 eluting second (58%). (Intermediate 3, 5-Br) ¹H NMR (400 MHz, d₆-DMSO) δ 8.77 (s, 1H), 8.01 (d, J=1.6 Hz, 1H), 7.76 (d, J=8.8 Hz, 1H), 7.56 (dd, J=8.8 and 1.6 Hz, 1H), 7.25 (s, 1H), 3.76 (s, 3H), 0.99 (s, 9H), 0.27 (s, 6H). (Intermediate 4, 6-Br) ¹H NMR (400 MHz, d₆-DMSO) δ 8.71 (s, 1H), 7.88 (d, J=1.6 Hz, 1H), 7.73 (d, J=8.8 Hz, 1H), 7.50 (dd, J=8.8 and 2.0 Hz, 1H), 7.26 (s, 1H), 3.77 (s, 3H), 0.99 (s, 9H), 0.26 (s, 6H).

### Intermediate 5: Methyl 5-(5-bromo-1H-benzimidazol-1-yl)-3-[(phenylmethyl)oxy]-2-thiophenecarboxylate

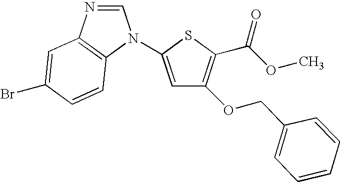

### Step A—Methyl 5-nitro-3-[(phenylmethyl)oxy]-2-thiophenecarboxylate

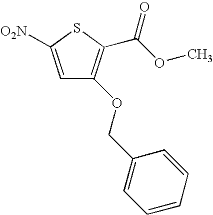

To a solution of methyl 3-hydroxy-5-nitro-2-thiophenecarboxylate, which may be prepared according to the procedure in J. Chem. Research (M), 2001, 1001-1004, (26.4 g, 130 mmol) in DMF (300 mL) was added K₂CO₃ (20.0 g, 145 mmol), followed by benzyl bromide (22.3 g, 130 mmol), and the reaction mixture was stirred at rt for 18 h. The solution was filtered to remove the solids, and the filtrate was poured slowly into 1 N HCl (600 mL). A yellow solid precipitated, and this solid was collected by vacuum filtration and was washed with water (3×300 mL) providing 37.0 g (97%) of the title compound. ¹H NMR (400 MHz, DMSO-d₆): δ 8.23 (s, 1H), 7.48-7.28 (m, 5H), 5.37 (s, 2H), 3.79 (s, 3H).

### Step B—Methyl 5-amino-3-[(phenylmethyl)oxy]-2-thiophenecarboxylate

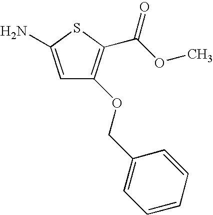

To a flask equipped with a temperature probe, an overhead mechanical stirrer, a reflux condenser, and an addition funnel was added iron powder (36.3 g, 650 mmol) and acetic acid (230 mL). The iron/acetic acid slurry was stirred mechanically and heated to an internal temperature of 50° C. To the addition funnel was added a solution of methyl 5-nitro-3-[(phenylmethyl)oxy]-2-thiophenecarboxylate (37.0 g, 126 mmol) in acetic acid (300 mL). The solution in the addition funnel was then added dropwise to the iron/acetic acid slurry at a rate such that the internal temperature was maintained at <60° C. (2.5 h total addition time). The reaction mixture was cooled to rt, and the entire mixture was then filtered through filter paper to remove insoluble material, rinsing with DCM (500 mL). The solution was concentrated to about 200 mL, rediluted with EtOAc (500 mL) and then quenched by addition of 6 N NaOH (250 mL) and saturated aqueous NaHCO₃ (200 mL). The aqueous and organic fractions were separated. The aqueous fraction was extracted with EtOAc (2×400 mL). The organic fractions were combined, dried over MgSO₄, filtered, and concentrated to afford 27.0 g (82%) of the title compound as a tan solid. ¹H NMR (400 MHz, DMSO-d₆): δ 7.42-7.26 (m, 5H), 6.78 (s, 2H), 5.76 (s, 1H), 5.10 (s, 2H), 3.56 (s, 3H); MS (ESI): 286 [M+Na]⁺.

### Step C—Methyl 5-[(4-bromo-2-nitrophenyl)amino]-3-[(phenylmethyl)oxy]-2-thiophenecarboxylate

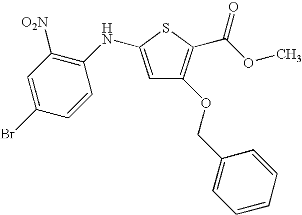

Methyl 5-amino-3-[(phenylmethyl)oxy]-2-thiophenecarboxylate (3.2 g, 12 mmol) and 1,4-dibromo-2-nitrobenzene (3.9 g, 14 mmol) were dissolved in 1,4-dioxane (100 mL). The solution was degassed for 15 min by bubbling N₂ through the stirring solution. XANTPHOS (0.32 g, 0.55 mmol), cesium carbonate (20 g, 63 mmol), and tris(dibenzylideneacetone) dipalladium(0) (0.23 g, 0.25 mmol) were added. The reaction was heated to 60° C. and stirred for 16 h. The reaction was cooled to rt and filtered through Celite. The solid was washed with 20% MeOH in DCM. Silica gel was added and the volatiles were evaporated under reduced pressure and the residue was purified by flash column chromatography (DCM to EtOAc) to give 3.9 g (70%) of the title compound as a solid. ¹H NMR (400 MHz, CDCl₃): δ 9.54 (s, 1H), 8.33 (s, 1H), 7.53-7.20 (m, 7H), 6.56 (s, 1H), 5.23 (s, 2H), 3.85 (s, 3H).

### Step D—Methyl 5-(5-bromo-1H-benzimidazol-1-yl)-3-[(phenylmethyl)oxy]-2-thiophenecarboxylate (title compound)

Methyl 5-[(4-bromo-2-nitrophenyl)amino]-3-[(phenylmethyl)oxy]-2-thiophenecarboxylate (3.9 g, 8.5 mmol) was dissolved in EtOAc (100 mL) with stirring. Sulfided platinum (5% weight on carbon, 1.3 g) was added, and the reaction was placed under 50 atm of H₂. After 16 h, additional sulfided platinum (5% weight on carbon, 1.3 g) was added, and the reaction was placed under 50 atm of H₂. After an additional 24 h, the reaction was filtered through a Celite pad washing with EtOAc. The filtrate was concentrated to afford 3.8 g of methyl 5-({2-amino-4-[(trifluoromethyl)oxy]phenyl}amino)-3-{[(1R)-1-(2-chlorophenyl)ethyl]oxy}-2-thiophenecarboxylate which was immediately dissolved in trimethyl orthoformate (50 mL) with stirring. Formic acid (1.0 mL, 26 mmol) was added and the reaction was stirred at 60° C. for 24 h. The volatiles were evaporated under reduced pressure and the residue was partitioned between DCM (200 mL) and water (100 mL). The layers were separated, and the organics were washed with water (3×50 mL), dried over MgSO₄ and filtered. Silica gel was added and the solvent evaporated under reduced pressure, and the residue was purified by flash column chromatography (Hexanes to EtOAc) to afford 3.3 g (87%) of the title compound as a solid. ¹H NMR (400 MHz, CDCl₃): δ 8.05 (d, 1H), 8.01-7.99 (m, 1H), 7.49-7.35 (m, 6H), 7.26 (s, 1H), 6.88 (s, 1H), 5.33 (s, 2H), 3.91 (s, 3H); MS (ESI): 443 & 445 [M+1 & M+3]⁺.

### Intermediate 6: Methyl 5-(5-bromo-1H-benzimidazol-1-yl)-3-hydroxy-2-thiophenecarboxylate

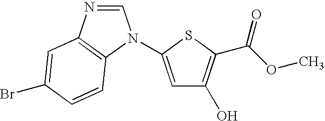

A solution of methyl 5-(5-bromo-1H-benzimidazol-1-yl)-3-[(phenylmethyl)oxy]-2-thiophenecarboxylate (1.5 g, 3.4 mmol) in 10 mL of TFA was heated to 50° C. After 6 h, the reaction was concentrated. The residue was dissolved in MeOH and neutralized with 7N ammonia in MeOH. The slurry was diluted with ether and filtered. The solid was washed with water and air-dried to give 1.02 g of the title compound (85%).

In an alternative procedure, to a solution of methyl 5-(5-bromo-1H-benzimidazol-1-yl)-3-{[tert-butyl(dimethyl)-silyl]oxy}thiophene-2-carboxylate (Intermediate 3, 11.8 g, 25 mmol) in 250 mL of THF cooled to 0° C. was added a 1M solution of tetrabutylammonium fluoride in THF (28 mL, 28 mmol). The reaction was quenched with water and extracted with EtOAc. The combined organic layers were washed with water, dried over MgSO₄ and concentrated onto silica gel. The crude material was purified by flash column chromatography (0-5% MeOH/DCM) to give the title compound.

¹H NMR (400 MHz, d₆-DMSO) δ 10.85 (s, 1H), 8.71 (s, 1H), 8.00 (s, 1H), 7.75 (d, J=8.8 Hz, 1H), 7.54 (d, J=8.4 Hz, 1H), 7.12 (s, 1H), 3.76 (s, 3H).

### Intermediate 7: Methyl 3-hydroxy-5-[5-(1-methyl-1H-pyrazol-4-yl)-1H-benzimidazol-1-yl]-2-thiophenecarboxylate

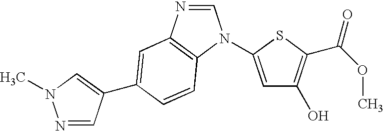

### Step A—Methyl 5-[5-(1-methyl-1H-pyrazol-4-yl)-1H-benzimidazol-1-yl]-3-[(phenylmethyl)oxy]-2-thiophenecarboxylate

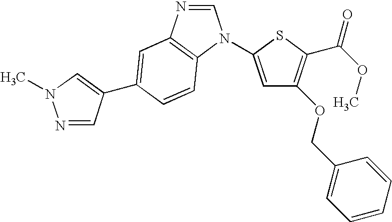

To a solution of methyl 5-(5-bromo-1H-benzimidazol-1-yl)-3-[(phenylmethyl)oxy]-2-thiophenecarboxylate (Intermediate 5, 2.8 g, 6.3 mmol) in DMA (60 mL) and 1N aqueous Na₂CO₃ (20 mL) was added 1-methyl-4-(4,4,5,5-tetramethyl-1,3,2-dioxaborolan-2-yl)-1H-pyrazole (1.6 g, 7.5 mmol), followed by Cl₂Pd(dppf) (0.60 g, 0.75 mmol), and the reaction mixture was heated to 80° C. for 1 h. The solution was filtered cooled to rt, diluted with EtOAc (250 mL) and washed with water (3×200 mL). The organic layer was dried over MgSO₄, filtered, and silica gel (10 g) was added. The volatiles were evaporated under reduced pressure, and the pre-adsorbed solids were loaded into a solid loading cartridge and subjected to a gradient elution using DCM (100%) to DCM: MeOH: ammonium hydroxide (90:10:1) using a RediSep silica gel cartridge (40 g; ISCO). The appropriate fractions were combined and concentrated under reduced pressure to give 1.6 g (3.6 mmol) of the title compound. ¹H NMR (400 MHz, CDCl₃): δ 8.03 (s, 1H), 7.90 (s, 1H), 7.79 (s, 1H), 7.64 (s, 1H), 7.54-7.32 (m, 7H), 6.88 (s, 1H), 5.32 (s, 2H), 3.96 (s, 3H), 3.90 (s, 3H).

### Step B—Methyl 3-hydroxy-5-[5-(1-methyl-1H-pyrazol-4-yl)-1H-benzimidazol-1-yl]-2-thiophenecarboxylate (title compound)

To methyl 5-[5-(1-methyl-1H-pyrazol-4-yl)-1H-benzimidazol-1-yl]-3-[(phenylmethyl)oxy]-2-thiophenecarboxylate (1.6 g, 3.6 mmol) was added TFA (20 mL) and the mixture was stirred at rt for 18 h. The solution was concentrated to give an oil and DCM (20 mL) was added resulting in the precipitation of a solid. The acid was neutralized by addition of 7N ammonia in MeOH and the solution diluted with DCM and MeOH so that all the solid dissolved. Silica gel (10 g) was added and the volatiles were evaporated under reduced pressure, and the pre-adsorbed solids were loaded into a solid loading cartridge and subjected to a gradient elution using DCM (100%) to DCM:MeOH:ammonium hydroxide (90:10:1) using a RediSep silica gel cartridge (40 g; ISCO). The appropriate fractions were combined and concentrated under reduced pressure to give 1.3 g (3.6 mmol) of the title compound as a white solid. ¹H NMR (400 MHz, DMSO-d₆): δ 8.64 (s, 1H), 8.17 (s, 1H), 7.96 (d, J=1.1 Hz, 1H), 7.91 (d, J=0.7 Hz, 1H), 7.74 (d, J=8.4 Hz, 1H), 7.60 (dd, J=8.4, 1.7 Hz, 1H), 7.11 (s, 1H), 3.84 (s, 3H), 3.76 (s, 3H).

### Alternate route to Intermediate 7, Step A: Methyl 5-[5-(1-methyl-1H-pyrazol-4-yl)-1H-benzimidazol-1-yl]-3-[(phenylmethyl)oxy]-2-thiophenecarboxylate

### Step A1-4-(1-Methyl-1H-pyrazol-4-yl)-2-nitroaniline

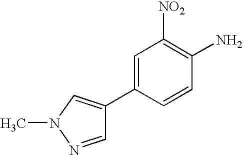

4-Bromo-2-nitroaniline (1.0 g, 4.6 mmol) and 1-methyl-4-(4,4,5,5-tetramethyl-1,3,2-dioxaborolan-2-yl)-1H-pyrazole (1.1 g, 5.1 mmol) were dissolved in 13 mL of DMA, placed under nitrogen, and heated to 80° C. A 2N aqueous solution of Na₂CO₃ was added, followed by Cl₂Pd(dppf) dichloromethane adduct (0.076 g, 0.9 mmol). Reaction was stirred at 80° C. for 1 h and then cooled to rt, poured into 150 mL of water and extracted with EtOAc (3×). Combined organics were dried over anhydrous MgSO₄, filtered, concentrated onto silica gel, and purified by flash chromatography using 0-50% EtOAc/hexanes. 4-(1-Methyl-1H-pyrazol-4-yl)-2-nitroaniline was isolated as a bright orange solid (1.0 g, 99%). MS (ESI): 219 [M+H]⁺.

### Step A2-4-(4-Iodo-3-nitrophenyl)-1-methyl-1H-pyrazole

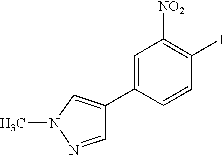

Iodine (12.4 g, 48.7 mmol), acetonitrile (50 mL), and tert-butyl nitrite (3.9 mL, 32.4 mmol) were combined under N₂ in a 3-neck round bottom flask fitted with reflux condenser and an addition funnel. The mixture was heated to 60° C. To the addition funnel was added a solution of 4-(1-methyl-1H-pyrazol-4-yl)-2-nitroaniline (1.0 g, 4.6 mmol) dissolved in DCM (100 mL) and methyl sulfoxide (10 mL). This solution was added dropwise over 15 min while heating at 60° C. During the last 2 min of the addition, bubbles of N₂ gas were observed. The reaction was stirred for an additional 2 h at 60° C. and then the heat was turned off and the reaction stirred at rt overnight. Aqueous sodium sulfite solution was added and the mixture was extracted with EtOAc (3×). Combined organic layers were dried over anhydrous MgSO₄, filtered, concentrated onto silica gel and purified by flash chromatography using 20-60% EtOAc/hexanes. 1.43 g (95%) of 4-(4-iodo-3-nitrophenyl)-1-methyl-1H-pyrazole was isolated as a yellow solid. MS (ESI): 330, 331 [M+H]⁺.

### Step A3—Methyl 5-{[4-(1-methyl-1H-pyrazol-4-yl)-2-nitrophenyl]amino}-3-[(phenylmethyl)oxy]-2-thiophenecarboxylate

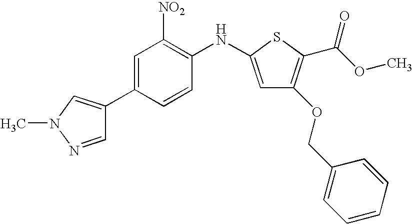

Methyl 5-amino-3-[(phenylmethyl)oxy]-2-thiophenecarboxylate (1.0 g, 3.8 mmol) and 4-(4-iodo-3-nitrophenyl)-1-methyl-1H-pyrazole (1.3 g, 3.8 mmol) were dissolved in anhydrous toluene (30 mL) and degassed with N₂ gas for 30 min. Cesium carbonate (6.2 g, 19.0 mmol) was added followed by XANTPHOS and trisdibenzylideneacetone palladium (II). The mixture was heated to 80° C. for 2 h and was then absorbed directly onto silica gel and flash chromatographed using 0-50% EtOAc/DCM. 1.62 g (98%) of methyl 5-{[4-(1-methyl-1H-pyrazol-4-yl)-2-nitrophenyl]amino}-3-[(phenylmethyl)oxy]-2-thiophenecarboxylate was isolated as a dark red/purple solid. MS (ESI): 465 [M+H]⁺.

### Step A4—Methyl 5-[5-(1-methyl-1H-pyrazol-4-yl)-1H-benzimidazol-1-yl]-3-[(phenylmethyl)oxy]-2-thiophenecarboxylate (title compound)

Methyl 5-{[4-(1-methyl-1H-pyrazol-4-yl)-2-nitrophenyl]amino}-3-[(phenylmethyl)oxy]-2-thiophenecarboxylate (1.0 g, 2.2 mmol) was dissolved in MeOH (30 mL). Trimethylorthoformate (6.0 mL, 53.8 mmol) was added followed by formic acid (0.81 mL, 21.5 mmol). Zinc dust (0.7 g, 10.7 mmol) was added and the reaction mixture was heated to 70° C. for 2 h and then cooled to rt. The reaction mixture was filtered through a pad of celite which was then washed with 20% MeOH/DCM. The crude reaction mixture was concentrated to remove the MeOH and the remaining mixture was poured into half-saturated aqueous NaHCO₃ solution and then extracted with a mixture of 4:1 DCM:i-PrOH. The combined organics were dried over anhydrous MgSO₄ and purified by flash chromatography to give 850 mg (89%) of the title compound, methyl 5-[5-(1-methyl-1H-pyrazol-4-yl)-1H-benzimidazol-1-yl]-3-[(phenylmethyl)oxy]-2-thiophenecarboxylate. MS (ESI): 445 [M+H]⁺

### Alternate route to Intermediate 7: Methyl 3-hydroxy-5-[5-(1-methyl-1H-pyrazol-4-yl)-1H-benzimidazol-1-yl]-2-thiophenecarboxylate

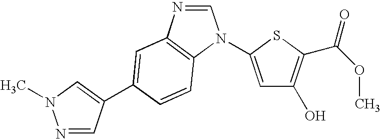

Methyl 5-(5-bromo-1H-benzimidazol-1-yl)-3-{[(1,1-dimethylethyl)(dimethyl) silyl]oxy}-2-thiophenecarboxylate (Intermediate 3, 20 g, 42.8 mmol) and 1-methyl-4-(4,4,5,5-tetramethyl-1,3,2-dioxaborolan-2-yl)-1H-pyrazole (11.12 g, 53.4 mmol) were dissolved in DMF (285 mL) with stirring in a flask equipped with an overhead stirrer, reflux condenser, and thermometer. The solution was degassed for 15 min by bubbling N₂ through the stirring solution. Cl₂Pd(dppf) (0.53 g, 0.73 mmol) was added followed by 1.6 M K₂CO₃ (142 mL). The reaction was heated to 80° C. and stirred for 2 h. The reaction was cooled to rt and transferred to 2 L flask. The mixture was acidified with acetic acid and then diluted with 1 L of water. The product was collected by filtration to give 14.3 g (94%) of the title compound as a solid. MS (ESI): 355 [M+H]⁺.

### Intermediate 8: Methyl 3-{[(1R)-1-(2-chloro-3-hydroxyphenyl)ethyl]oxy}-5-[5-(1-methyl-1H-pyrazol-4-yl)-1H-benzimidazol-1-yl]-2-thiophenecarboxylate

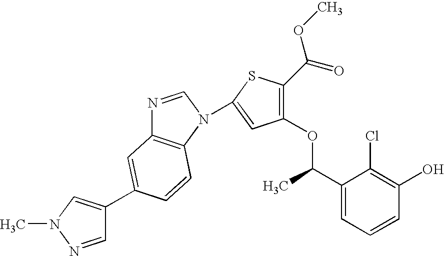

### Step A—Methyl 3-{[(1R)-1-(2-chloro-3-{[(1,1-dimethylethyl)(dimethyl)silyl]oxy}phenyl)ethyl]oxy}-5-[5-(1-methyl-1H-pyrazol-4-yl)-1H-benzimidazol-1-yl]-2-thiophenecarboxylate

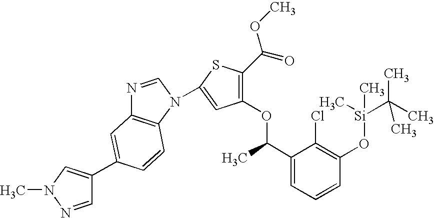

To a slurry of methyl 3-hydroxy-5-[5-(1-methyl-1H-pyrazol-3-yl)-1H-benzimidazol-1-yl]-2-thiophenecarboxylate (Intermediate 7, 0.71 g, 2.0 mmol) and (1S)-1-(2-chloro-3-{[(1,1-dimethylethyl)(dimethyl)silyl]oxy}phenyl)ethanol (Intermediate 2, 0.63 g, 2.2 mmol) in DCM (20 mL) was added triphenylphosphine (1.1 g, 4.0 mmol) and di-tert-butyl azodicarboxylate (0.92 g, 4.0 mmol). The clear, yellow solution was stirred 1 h then silica (5 g) was added. The volatiles were evaporated under reduced pressure and the pre-adsorbed solids were loaded into a solid loading cartridge and subjected to a gradient elution using DCM (100%) to DCM:MeOH:ammonium hydroxide (90:10:1) using a RediSep silica gel cartridge (40 g; ISCO). The appropriate fractions were combined and concentrated under reduced pressure to give 1.1 g (1.8 mmol) of the title compound as a white solid. ¹H NMR (400 MHz, CDCl₃): δ 7.98 (s, 1H), 7.87 (s, 1H), 7.78 (s, 1H), 7.64 (s, 1H), 7.46-7.44 (m, 2H), 7.26-7.23 (m, 1H), 7.16 (dd, J=7.9, 7.8 Hz 1H), 6.85-6.83 (m, 1H), 5.82 (q, J=6.3 Hz, 1H), 3.96 (s, 3H), 3.91 (s, 3H), 1.72 (d, J=6.3 Hz, 3H), 1.01 (s, 9H), 0.21 (s, 3H), 0.19 (s, 3H).

### Step B—Methyl 3-{[(1 R^{A})-1-(2-chloro-3-hydroxyphenyl)ethyl]oxy}-5-[5-(1-methyl-1H-pyrazol-4-yl)-1H-benzimidazol-1-yl]-2-thiophenecarboxylate (title compound)

To a solution of methyl 3-{[(1R)-1-(2-chloro-3-{[(1,1-dimethylethyl)(dimethyl)silyl]oxy}phenyl)ethyl]oxy}-5-[5-(1-methyl-1H-pyrazol-4-yl)-1H-benzimidazol-1-yl]-2-thiophenecarboxylate (0.72 g, 1.2 mmol) in THF (5 mL) was added a solution of 1N tetrabutylammonium fluoride in THF (1.4 mL, 1.4 mmol). After 10 min, silica (5 g) was added, the volatiles were evaporated under reduced pressure and the pre-adsorbed solids were loaded into a solid loading cartridge and subjected to a gradient elution using DCM (100%) to DCM:MeOH:ammonium hydroxide (80:20:1) using a RediSep silica gel cartridge (12 g; ISCO). The appropriate fractions were combined and concentrated under reduced pressure to give 0.53 g (1.0 mmol) of the title compound as a light yellow foam. ¹H NMR (400 MHz, CDCl₃): δ 7.97 (s, 1H), 7.87 (s, 1H), 7.78 (s, 1H), 7.63 (s, 1H), 7.46-7.44 (m, 1H), 7.36 (d, J=7.8 Hz, 1H), 7.24-7.20 (m, 2H), 7.01-6.97 (m, 1H), 6.64 (s, 1H) 5.73 (q, J=6.4 Hz, 1H), 3.95 (s, 3H), 3.91 (s, 3H), 1.73 (d, J=6.4 Hz, 3H).

### Intermediate 9: Methyl 5-(5-bromo-1H-benzimidazol-1-yl)-3-{[(1R)-1-(2-chloro-3-{[(1,1-dimethylethyl)(dimethyl)silyl]oxy}phenyl)ethyl]oxy}-2-thiophenecarboxylate

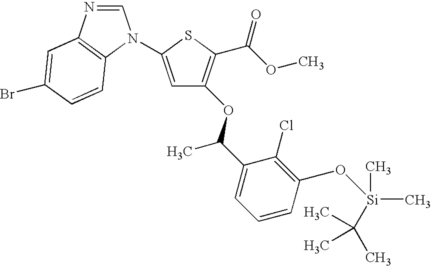

Methyl 5-(5-bromo-1H-benzimidazol-1-yl)-3-hydroxy-2-thiophenecarboxylate (Intermediate 6, 1.02 g, 2.9 mmol) and (1S)-1-(2-chloro-3-{[(1,1-dimethylethyl)(dimethyl)silyl]oxy}phenyl)-ethanol (Intermediate 2, 1.0 g, 3.5 mmol) were coupled using a procedure analogous to Intermediate 8, Step A to give 1.6 g of the title compound (89%). ¹H NMR (400 MHz, d₆-DMSO) δ 8.69 (s, 1H), 8.00 (s, J=1.6 Hz, 1H), 7.59 (d, J=8.8 Hz, 1H), 7.50 (dd, J=8.8 and 1.6 Hz, 1H), 7.34-7.28 (m, 3H), 6.96 (dd, J=6.8 and 2.8 Hz, 1H), 5.93 (m, 1H), 3.81 (s, 3H), 1.60 (d, J=6.0 Hz, 3H), 0.94 (s, 9H), 0.17 (s, 3H), 0.13 (s, 3H).

### Intermediate 10: Methyl 5-[5,6-bis(methyloxy)-1H-benzimidazol-1-yl]-3-{[(1R)-1-(2-chloro-3-hydroxyphenyl)ethyl]oxy}-2-thiophenecarboxylate

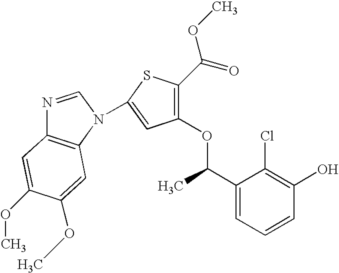

### Step A—Methyl 5-[5,6-bis(methyloxy)-1H-benzimidazol-1-yl]-3-{[(1R)-1-(2-chloro-3-{[(1,1-dimethylethyl)(dimethyl)silyl]oxy}phenyl)ethyl]oxy}-2-thiophenecarboxylate

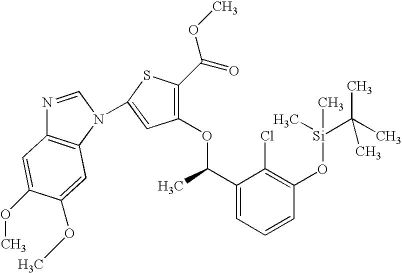

Methyl 5-[5,6-bis(methyloxy)-1H-benzimidazol-1-yl]-3-hydroxy-2-thiophenecarboxylate (which can be synthesized following the procedure found in PCT Int. Appl. WO 2004073612) (3.3 g, 10 mmol) and (1S)-1-(2-chloro-3-{[(1,1-dimethylethyl)(dimethyl)silyl]oxy}phenyl)ethanol (Intermediate 2, 2.9 g, 10 mmol) were coupled using a procedure analogous to Intermediate 8, Step A to give 4.8 g of the desired product (80%). ¹H NMR (400 MHz, CDCl₃): δ 7.85 (s, 1H), 7.27-7.14 (m, 3H), 6.92 (s, 1H), 6.82 (d, J=8.0 Hz, 1.6, 1H), 6.64 (s, 1H), 5.80 (q, J=6.4 Hz, 1H), 3.94 (s, 3H), 3.92 (s, 3H), 3.88 (s, 3H), 1.72 (d, J=6.4 Hz, 3H).

### Step B—Methyl 5-[5,6-bis(methyloxy)-1H-benzimidazol-1-yl]-3-{[(1R)-1-(2-chloro-3-hydroxyphenyl)ethyl]oxy}-2-thiophenecarboxylate (title compound)

Methyl 5-[5,6-bis(methyloxy)-1H-benzimidazol-1-yl]-3-{[(1R)-1-(2-chloro-3-{[(1,1-dimethylethyl)(dimethyl)silyl]oxy}phenyl)ethyl]oxy}-2-thiophenecarboxylate (4.8 g, 8.0 mmol) was deprotected using a procedure analogous to Intermediate 8, Step B to give 2.0 g (51%) of the title compound. ¹H NMR (400 MHz, DMSO): δ 10.29 (s, 1H), 8.42 (s, 1H), 7.33 (s, 1H), 7.24 (s, 1H), 7.19 (dd, J=8.0, 7.8 Hz, 1H) 7.10 (dd, J=7.8, 1.4 Hz, 1H), 7.06 (s, 1H), 6.90 (dd, J=8.0, 1.4, 1H), 5.97 (q, J=6.4 Hz, 1H), 3.82 (s, 3H), 3.81 (s, 3H), 3.80 (s, 3H), 1.61 (d, J=6.4 Hz, 3H).

### Intermediate 11: Methyl 5-(1H-benzimidazol-1-yl)-3-[((1R)-1-{3-[(2-bromoethyl)oxy]-2-chlorophenyl}ethyl)oxy]-2-thiophenecarboxylate

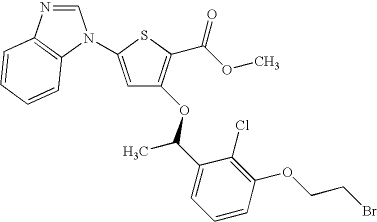

### Step A: Methyl 5-(1H-benzimidazol-1-yl)-3-{[(1R)-1-(2-chloro-3-hydroxyphenyl)ethyl]oxy}-2-thiophenecarboxylate

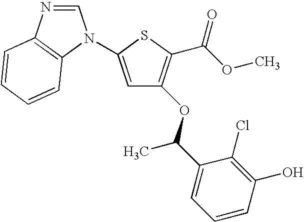

Title compound (2.2 g) was prepared from methyl 5-(1H-benzimidazol-1-yl)-3-hydroxy-2-thiophenecarboxylate (J. Heterocyclic Chem., 1987, 24, 1301-1303) (1.9 g, 7.0 mmol) and (1S)-1-(2-chloro-3-{[(1,1-dimethylethyl)(dimethyl)silyl]oxy}phenyl)ethanol (Intermediate 2, 2.0 g, 7.0 mmol) using a procedure analogous to Intermediate 8.

### Step B: Methyl 5-(1H-benzimidazol-1-yl)-3-[((1R)-1-{3-[(2-bromoethyl)oxy]-2-chlorophenyl}ethyl)oxy]-2-thiophenecarboxylate (title compound)

Methyl 5-(1H-benzimidazol-1-yl)-3-{[(1R)-1-(2-chloro-3-hydroxyphenyl)ethyl]oxy}-2-thiophenecarboxylate (600 mg, 1.4 mmol) and 2-bromoethanol (120 μL, 1.7 mmol) were coupled using a procedure analogous to Intermediate 8, Step A to give 529 mg of the title compound (71%). ¹H NMR (400 MHz, d₆-DMSO) δ 8.65 (s, 1H), 7.77 (d, J=7.2 Hz, 1H), 7.64 (d, J=8.0 Hz, 1H), 7.39-7.31 (m, 5H), 7.10 (dd, J=8.0 and 1.6 Hz, 1H), 5.98 (m, 1H), 4.40-4.37 (m, 2H), 3.81-3.79 (m, 5H), 1.61 (d, J=6.0 Hz, 3H).

### Intermediate 12: 1-(2-Chloro-5-iodophenyl)ethanol

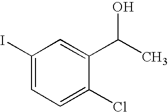

### Step A—2-Chloro-5-iodo-N-methyl-N-(methyloxy)benzamide

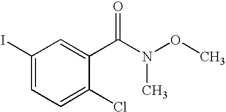

To a mixture of 2-chloro-5-iodobenzoic acid (5.0 g, 17.7 mmol) and N,O-dimethylhydroxylamine hydrochloride (1.90 g, 19.5 mmol) in DCM was added diisopropylamine (3.4 mL, 19.5 mmol). When everything went into solution, 1,3-dicyclohexylcarbodiimide (3.65 g, 17.7 mmol) was added and a white precipitate formed. TLC showed the starting material to be consumed and the reaction mixture was diluted with diethyl ether. The white solid was filtered and washed thoroughly with diethyl ether. The filtrate was concentrated and purified by column chromatography (10-15% EtOAc and hexanes) to give 4.68 g (81%) of the desired product. ¹H NMR (400 MHz, d₆-DMSO) δ 7.80-7.74 (m, 2H), 7.28 (d, J=8.4 Hz, 1H), 3.42 (s, 3H), 3.24 (s, 3H).

### Step B—2-Chloro-5-iodobenzaldehyde

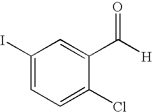

To a solution of 2-chloro-5-iodo-N-methyl-N-(methyloxy)benzamide (4.68 g, 14.4 mmol) in 100 ml toluene cooled to −78° C. was added a 1M solution of diisobutylaluminum hydride (17.3 mL, 17.3 mmol). When TLC showed the starting material to be consumed, the reaction was quenched with methanol and warmed to ambient temperature. Rochelle's salt solution was added and the cloudy mixture was stirred for several hours. The 2 layers were separated. The aqueous phase was extracted with DCM. The combined organic layers were washed with water and brine, dried over MgSO₄ and concentrated to give 3.82 g (99%) of a white solid. ¹H NMR (400 MHz, d₆-DMSO) δ 10.19 (s, 1H), 8.08 (d, J=2.0 Hz, 1H), 8.01 (dd, J=8.4 and 2.0 Hz, 1H), 7.42 (d, J=8.4 Hz, 1H).

### Step C—1-(2-Chloro-5-iodophenyl)ethanol (title compound)

To a solution of 2-chloro-5-iodobenzaldehyde (3.82 g, 14.3 mmol) in 100 mL of THF cooled to −78° C. was added a 3M solution of methyl magnesium bromide in THF (5.2 mL, 15.7 mmol). When TLC showed the starting material to be consumed the reaction was quenched with water and allowed to warm to rt. The aqueous solution was extracted with EtOAc. The combined organic layers were washed with brine, dried over MgSO₄ and concentrated. The crude material was purified by column chromatography to give the title compound as a white solid. ¹H NMR (400 MHz, d₆-DMSO) δ 7.85 (d, J=2.0 Hz, 1H), 7.57 (dd, J=8.0 and 2.0 Hz, 1H), 7.15 (d, J=8.0 Hz, 1H), 5.46 (d, J=4.4 Hz, 1H), 4.90 (m, 1H), 1.26 (d, J=6.4 Hz, 3H).

### Example 1

### 5-[5,6-Bis(methyloxy)-1H-benzimidazol-1-yl]-3-({(1R)-1-[2-chloro-5-({[2-(dimethylamino)ethyl]amino}carbonyl)phenyl]ethyl}oxy)-2-thiophenecarboxamide formate

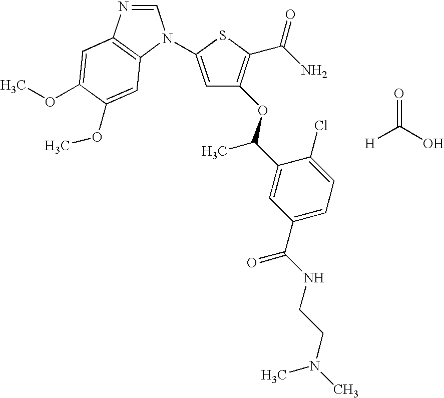

### Step A—Methyl 5-[5,6-bis(methyloxy)-1H-benzimidazol-1-yl]-3-{[1-(2-chloro-5-iodophenyl)ethyl]oxy}-2-thiophenecarboxylate

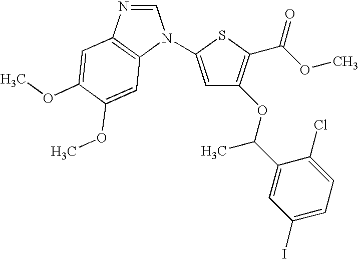

Methyl 5-[5,6-bis(methyloxy)-1H-benzimidazol-1-yl]-3-hydroxy-2-thiophenecarboxylate (which can be synthesized following the procedure found in PCT Int. Appl. WO 2004073612) (3.3 g, 9.8 mmol) and 1-(2-chloro-5-iodophenyl)ethanol (Intermediate 12, 3.33 g, 11.8 mmol) were coupled according to the procedure analogous to Intermediate 8, Step A to give 4.24 g (72%) of the desired product. ¹H NMR (400 MHz, d₆-DMSO) δ 8.43 (s, 1H), 8.10 (d, J=2.0 Hz, 1H), 7.66 (dd, J=8.6 and 2.2 Hz, 1H), 7.52 (s, 1H), 7.31 (s, 1H), 7.24 (d, J=8.4 Hz, 1H), 7.16 (s, 1H), 5.91 (m, 1H), 3.85 (s, 3H), 3.81 (s, 3H), 3.79 (s, 3H), 1.59 (d, J=6.0 Hz, 3H).

### Step B—5-[5,6-Bis(methyloxy)-1H-benzimidazol-1-yl]-3-{[(1R)-1-(2-chloro-5-iodophenyl)ethyl]oxy}-2-thiophenecarboxamide

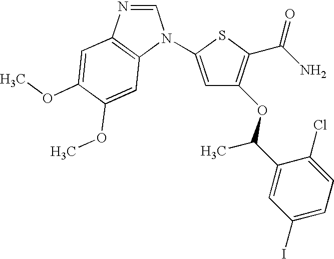

A solution of methyl 5-[5,6-bis(methyloxy)-1H-benzimidazol-1-yl]-3-{[1-(2-chloro-5-iodophenyl)ethyl]oxy}-2-thiophenecarboxylate (4.2 g, 7.0 mmol) in 7N ammonia in MeOH was heated in a sealed tube at 100° C. After 16 h, the reaction was cooled to rt and concentrated to a yellow solid. The solid was triturated in DCM, filtered and dried to give 3.02 g (74%) of the desired product. The enantiomers were separated using packed column supercritical fluid chromatography (SFC) with a method of 20% MeOH+10% CHCl₃ in CO₂, 90 g/min, 102 bar, 27° C. on a 3×25 cm Diacel OJ-H column. The desired product was the second enantiomer to elute. ¹H NMR (400 MHz, d₆-DMSO) δ 8.34 (s, 1H), 8.05 (s, J=2.0 Hz, 1H), 7.81 (s, 1H), 7.78 (dd, J=8.4 and 2.4 Hz, 1H), 7.31 (s, 1H), 7.26 (d, J=8.4 Hz, 1H), 7.20 (s, 1H), 7.13 (s, 1H), 7.07 (s, 1H), 5.92 (m, 1H), 3.80 (s, 3H), 3.79 (s, 3H), 1.69 (d, J=6.0 Hz, 3H).

### Step C—5-[5,6-Bis(methyloxy)-1H-benzimidazol-1-yl]-3-{[(1R)-1-(2-chloro-5-{[(2,5-dioxo-1-pyrrolidinyl)oxy]carbonyl}phenyl)ethyl]oxy}-2-thiophenecarboxamide

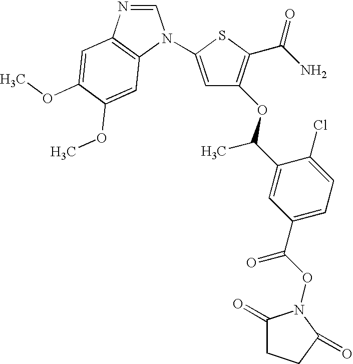

5-[5,6-Bis(methyloxy)-1H-benzimidazol-1-yl]-3-{[1-(2-chloro-5-iodophenyl)ethyl]oxy}-2-thiophenecarboxamide (696 mg, 1.19 mmol), N-hydroxysuccinimide (192 mg, 1.67 mmol), palladium (II) acetate (13 mg, 0.0595 mmol) and XANTPHOS (34 mg, 0.0595 mmol) were placed into a round-bottom flask and diluted into 5 mL of DMSO. Triethylamine (0.25 mL, 1.78 mmol) was added and the mixture was degassed with CO. The reaction was heated at 70° C. for 16 h under a balloon of CO. The reaction was cooled to ambient temperature and diluted with DCM. The organic solution was washed with water and saturated NaHCO₃, dried over MgSO₄ and concentrated. The residue was triturated with DCM and ether to give the desired product. ¹H NMR (400 MHz, d₆-DMSO) δ 8.32 (t, J=2.4 Hz, 1H), 8.03 (dd, J=8.4 and 2.4 Hz, 1H), 7.77 (d, J=8.4 Hz, 1H), 7.28 (s, 1H), 7.21 (br s, 1H), 7.14 (s, 1H), 7.06 (s, 1H), 6.03 (m, 1H), 3.78 (s, 3H), 3.76 (s, 3H), 1.74 (d, J=6.4 Hz, 3H).

### Step D—5-[5,6-Bis(methyloxy)-1H-benzimidazol-1-yl]-3-({(1R)-1-[2-chloro-5-({[2-(dimethylamino)ethyl]amino}carbonyl)phenyl]ethyl}oxy)-2-thiophenecarboxamide formate (title compound)

To a solution of 5-[5,6-bis(methyloxy)-1H-benzimidazol-1-yl]-3-{[(1R)-1-(2-chloro-5-{[(2,5-dioxo-1-pyrrolidinyl)oxy]carbonyl}phenyl)ethyl]oxy}-2-thiophenecarboxamide (90 mg, 0.15 mmol) in DCM was added N,N-dimethylethylenediamine (21 μL, 0.19 mmol) and triethylamine (63 μL, 0.45 mmol). When TLC showed the consumption of starting material, the reaction was diluted with DCM and washed 3 times with water. The organic phase was dried over MgSO₄ and concentrated. The crude material was purified by reverse phase LC to give 32 mg (37%) of the title compound. ¹H NMR (400 MHz, d₆-DMSO) δ 8.52 (m, 1H), 8.33 (s, 1H), 8.14 (m, 2H), 7.85 (s, 1H), 7.77 (dd, J=8.4 and 1.6 Hz, 1H), 7.57 (d, J=8.4 Hz, 1H), 7.29 (s, 1H), 7.12 (s, 1H), 7.09 (s, 1H), 7.04 (s, 1H), 6.00 (m, 1H), 3.79 (s, 3H), 3.77 (s, 3H), 3.33 (m, 2H), 2.42 (t, J=6.8 Hz, 2H), 2.19 (s, 6H), 1.72 (d, J=6.4 Hz, 3H). HRMS calculated C₂₇H₃₀ClN₅O₅S 572.1734, found 572.1731.

### Example 2

### 5-[5,6-Bis(methyloxy)-1H-benzimidazol-1-yl]-3-[((1R)-1-{2-chloro-3-[(2-hydroxyethyl)amino]phenyl}ethyl)oxy]-2-thiophenecarboxamide

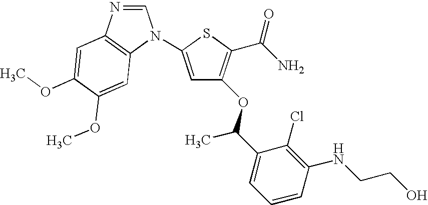

### Step A—Methyl 5-[5,6-bis(methyloxy)-1H-benzimidazol-1-yl]-3-{[(1R)-1-(2-chloro-3-nitrophenyl)ethyl]oxy}-2-thiophenecarboxylate

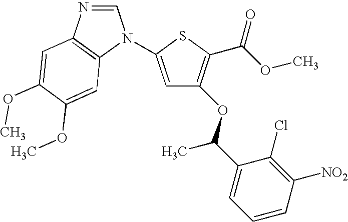

To a solution of methyl 5-[5,6-bis(methyloxy)-1H-benzimidazol-1-yl]-3-hydroxy-2-thiophenecarboxylate (which can be synthesized following the procedure found in PCT Int. Appl. WO 2004073612) (1.14 g, 3.4 mmol) and (1S)-1-(2-chloro-3-nitrophenyl)ethanol (Intermediate 1, 822 mg, 4.1 mmol) in 30 mL of DCM was added polymer supported-triphenylphosphine (3 g, 6.8 mmol) and di-tert-butyl azodicarboxylate (1.6 g, 6.8 mmol). After 16 h, the reaction mixture was filtered, and the resin was rinsed with alternating DCM and MeOH. The filtrate was concentrated and purified by flash column chromatography (10-20% EtOAc:hexanes) to give 1.4 g of the desired product (80%). ¹H NMR (400 MHz, d₆-DMSO) δ 8.42 (s, 1H), 8.03 (d, J=8.0 Hz, 1H), 7.98 (d, J=8.0 Hz, 1H), 7.68 (t, J=8.0 Hz, 1H), 7.50 (s, 1H), 7.30 (s, 1H), 7.14 (s, 1H), 6.06 (m, 1H), 3.81 (s, 3H), 3.79 (s, 6H), 1.64 (d, J=6.4 Hz, 3H).

### Step B—5-[5,6-Bis(methyloxy)-1H-benzimidazol-1-yl]-3-{[(1R)-1-(2-chloro-3-nitrophenyl)ethyl]oxy}-2-thiophenecarboxamide

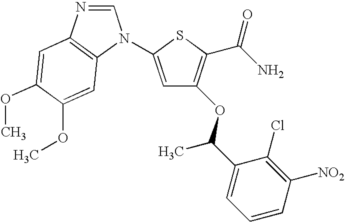

A mixture of methyl 5-[5,6-bis(methyloxy)-1H-benzimidazol-1-yl]-3-{[(1R)-1-(2-chloro-3-nitrophenyl)ethyl]oxy}-2-thiophenecarboxylate (1.4 g, 2.7 mmol) in 7 N ammonia in MeOH in a sealed tube was heated at 80° C. After 16 h, the reaction was cooled to rt. The precipitate was filtered, rinsed with ether and dried to give 1.05 g of the desired product (77%). ¹H NMR (400 MHz, d₆-DMSO) δ 8.32 (s, 1H), 7.99-7.96 (m, 2H), 7.80 (br s, 1H), 7.65 (t, J=8.0 Hz, 1H), 7.28 (s, 1H), 7.18 (s, 1H), 7.13 (br s, 1H), 7.06 (s, 1H), 6.06 (m, 1H), 3.78 (s, 3H), 3.76 (s, 3H), 1.72 9d, J=6.4 Hz, 3H).

### Step C—3-{[(1R)-1-(3-amino-2-chlorophenyl)ethyl]oxy}-5-[5,6-bis(methyloxy)-1H-benzimidazol-1-yl]-2-thiophenecarboxamide

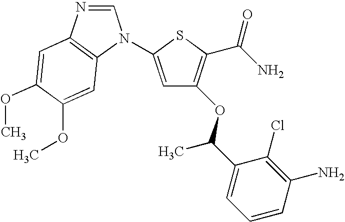

To 5-[5,6-bis(methyloxy)-1H-benzimidazol-1-yl]-3-{[(1R)-1-(2-chloro-3-nitrophenyl)ethyl]oxy}-2-thiophenecarboxamide (1.04 g, 2.0 mmol) and iron powder (0.56 g, 10 mmol) was added acetic acid (6 mL, 100 mmol). The dark mixture was heated at 50° C. After 30 min, EtOAc was added and 5N NaOH was added to neutralize the mixture. The mixture was filtered through a pad of celite. The organic layer was separated, dried over MgSO₄ and concentrated to give 0.47 g of the desired product (50%). ¹H NMR (400 MHz, d₆-DMSO) δ 8.32 (s, 1H), 7.78 (br s, 1H), 7.28 (s, 1H), 7.06-6.97 (m, 4H), 6.74-6.68 (m, 2H), 5.89 (m, 1H), 5.44 (s, 2H), 3.77 (s, 3H), 3.75 (s, 3H), 1.64 (d, J=6.4 Hz, 3H).

### Step D—5-[5,6-Bis(methyloxy)-1H-benzimidazol-1-yl]-3-[((1R)-1-{2-chloro-3-[(2-hydroxyethyl)amino]phenyl}ethyl)oxy]-2-thiophenecarboxamide (title compound)

To a solution of 3-{[(1R)-1-(3-amino-2-chlorophenyl)ethyl]oxy}-5-[5,6-bis(methyloxy)-1H-benzimidazol-1-yl]-2-thiophenecarboxamide (118 mg, 0.25 mmol) in 5 mL of 1,2-dichloroethane was added glycolaldehyde (23 mg, 0.375 mmol), acetic acid (29 μL, 0.50 mmol) and sodium triacetoxyborohydride (106 mg, 0.50 mmol), and the reaction was heated at 60° C. After 16 h, the reaction was diluted with DCM and washed with saturated NaHCO₃ and water. The organic phase was dried over MgSO₄ and purified by silica gel chromatography to give 8 mg (6%) of the title compound. ¹H NMR (400 MHz, d₄-CD₃OD) δ 8.14 (s, 1H), 7.19-7.15 (m, 2H), 6.85 (m, 2H), 6.80 (d, J=7.6 Hz, 1H), 6.69 (d, J=8.0 Hz, 1H), 5.96 (m, 1H), 3.85 (s, 3H), 3.76 (s, 3H), 3.73 (t, J=5.6 Hz, 2H), 3.26 (t, J=5.6 Hz, 2H), 1.73 (d, J=6.4 Hz, 3H).

### Example 3

### 3-[((1R)-1-{3-[(2-aminoethyl)oxy]-2-chlorophenyl}ethyl)oxy]-5-[5,6-bis(methyloxy)-1H-benzimidazol-1-yl]-2-thiophenecarboxamide

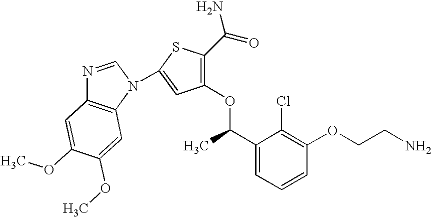

### Step A—Methyl 5-[5,6-bis(methyloxy)-1H-benzimidazol-1-yl]-3-[((1R)-1-{3-[(2-bromoethyl)oxy]-2-chlorophenyl}ethyl)oxy]-2-thiophenecarboxylate

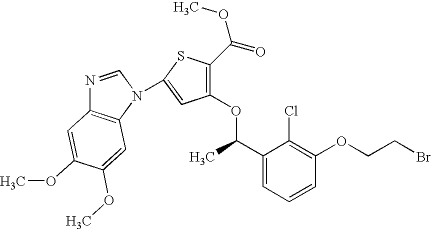

To methyl 5-[5,6-bis(methyloxy)-1H-benzimidazol-1-yl]-3-{[(1R)-1-(2-chloro-3-hydroxyphenyl)ethyl]oxy}-2-thiophenecarboxylate (Intermediate 10, 0.98 g, 2.0 mmol) and K₂CO₃ (0.55 g, 4.0 mmol) in DMF (50 mL) was added 1,2-dibromoethane (0.94 g, 5.0 mmol), and the mixture was stirred for 18 h. The solution was filtered then poured into 0.1 N HCl (200 mL). The product was extracted with DCM (2×200 mL), dried over MgSO₄, filtered and concentrated onto silica. The mixture was purified by flash column chromatography (0-100% EtOAc:hexanes) to give 0.20 g of the desired product (17%). ¹H NMR (400 MHz, CDCl₃): δ 7.84 (s, 1H), 7.30-7.22 (m, 3H), 6.93 (s, 1H), 6.83 (dd, J=8.0, 1.6 Hz, 1H), 6.63 (s, 1H), 5.82 (q, J=6.4 Hz, 1H), 4.30 (t, J=6.4 Hz, 2H), 3.92 (s, 3H), 3.90 (s, 3H), 3.88 (s, 3H), 3.66 (t, J=6.4 Hz, 2H), 1.71 (d, J=6.4 Hz, 3H) MS (ESI): 595 & 597 [M+H]⁺.

### Step B—3-[((1R)-1-{3-[(2-aminoethyl)oxy]-2-chlorophenyl}ethyl)oxy]-5-[5,6-bis(methyloxy)-1H-benzimidazol-1-yl]-2-thiophenecarboxamide (title compound)

Methyl 5-[5,6-bis(methyloxy)-1H-benzimidazol-1-yl]-3-[((1R)-1-{3-[(2-bromoethyl)oxy]-2-chlorophenyl}ethyl)oxy]-2-thiophenecarboxylate (0.10 g, 0.17 mmol) was heated at 100° C. for 18 h with NH₃ (5 mL of 7N in MeOH) in a sealed tube. The reaction was cooled, and then the solvent was removed under vacuum. The mixture was purified by flash column chromatography (100%-80:20:1 DCM:MeOH:ammonium hydroxide) to give 0.026 g of the desired product (30%). ¹H NMR (400 MHz, CDCl₃): δ 7.81 (s, 1H), 7.27-7.22 (m, 2H), 7.20 (bs, 1H), 7.05 (d, J=7.6 Hz, 1H) 6.93 (s, 1H), 6.88 (d, J=8.3 Hz, 1H), 6.54 (s, 1H), 5.92 (bs, 1H) 5.86 (q, J=6.4 Hz, 1H), 4.08-3.99 (m, 2H), 3.92 (s, 3H), 3.86 (s, 3H), 3.20-3.08 (m, 2H), 1.73 (d, J=6.4 Hz, 3H), 1.64 (bs, 2H). MS (ESI): 517 [M+H]⁺.

### Example 4

### 3-[((1R)-1-{3-[(2-Aminoethyl)oxy]-2-chlorophenyl}ethyl)oxy]-5-(1H-benzimidazol-1-yl)-2-thiophenecarboxamide

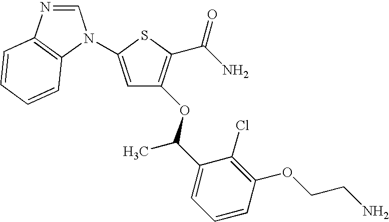

A solution of methyl 5-(1H-benzimidazol-1-yl)-3-[((1R)-1-{3-[(2-bromoethyl)oxy]-2-chlorophenyl}ethyl)oxy]-2-thiophenecarboxylate (Intermediate 11,125 mg, 0.23 mmol) in 7N ammonia in MeOH in a sealed tube was heated at 90° C. After 72 h, the reaction was cooled to rt and concentrated onto silica gel. The crude material was purified by column chromatography (0-100% 10% MeOH/DCM+1% NH₄OH and DCM) to give 56 mg (53%) of the title compound. ¹H NMR (400 MHz, d₆-DMSO) δ 8.55 (s, 1H), 7.80 (br s, 1H), 7.76-7.74 (m, 1H), 7.51-7.48 (m, 1H), 7.36-7.31 (m, 3H), 7.20 (d, J=7.6 Hz, 1H), 7.12-7.09 (m, 3H), 5.99 (m, 1H), 3.98 (t, J=5.8 Hz, 2H), 2.87 (t, J=5.8 Hz, 2H), 1.70 (d, J=6.4 Hz, 3H). HRMS calculated C₂₂H₂₂ClN₄O₃S 457.1101, found 457.1103.

### Example 5

### 3-[((1 R-1-{2-chloro-3-[(3-hydroxypropyl)oxy]phenyl}ethyl)oxy]-5-[5-(1-methyl-1H-pyrazol-4-yl)-1H-benzimidazol-1-yl]-2-thiophenecarboxamide

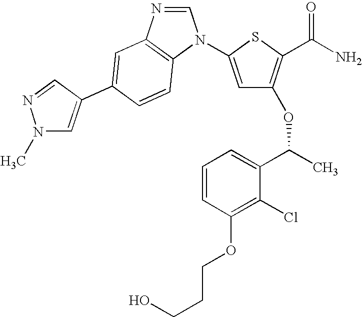

Methyl 3-{[(1R)-1-(2-chloro-3-hydroxyphenyl)ethyl]oxy}-5-[5-(1-methyl-1H-pyrazol-4-yl)-1H-benzimidazol-1-yl]-2-thiophenecarboxylate (Intermediate 8, 0.10 g, 0.20 mmol), 3-bromo-1-propanol (0.033 g, 0.24 mmol) and K₂CO₃ (0.055 g, 0.40 mmol) were combined in DMF (1 mL) and stirred at 80° C. for 16 h. The mixture was cooled, diluted with EtOAc, and partitioned with water/brine (1:1). The organic phase was washed with brine, then dried over Na₂SO₄, filtered, and concentrated to give 0.12 g of a yellow residue. The residue was combined with 3 mL of ammonia in MeOH (7N) in a sealed vessel and heated while stirring at 95° C. behind a blast shield for 16 h. The mixture was cooled and filtered to give 0.062 g (56%) of the title compound as a white solid. ¹H NMR (400 MHz, DMSO-d₆) δ 8.52 (s, 1H), 8.17 (s, 1H), 7.93 (s, 1H), 7.90 (s, 1H), 7.79 (s, 0H), 7.54 (d, J=8.0 Hz, 1H), 7.47 (d, J=8.3 Hz, 1H), 7.34 (t, J=8.0 Hz, 1H), 7.19 (d, J=7.8 Hz, 1H), 7.11 (s, 1H), 7.09 (d, J=8.0 Hz, 1H), 5.98 (q, J=6.3 Hz, 1H), 4.53 (t, J=5.2 Hz, 1H), 4.11-4.08 (m, 2H), 3.84 (s, 3H), 3.55 (m, 2H), 1.85 (quint, J=6.2 Hz, 2H), 1.69 (d, J=7.0 Hz, 3H); MS (ESI) m/z 552.22 (M+H)⁺.

### Example 6

### 3-[((1R)-1-{2-chloro-3-[(2-hydroxyethyl)oxy]phenyl}ethyl)oxy]-5-[5-(1-methyl-1H-pyrazol-4-yl)-1H-benzimidazol-1-yl]-2-thiophenecarboxamide

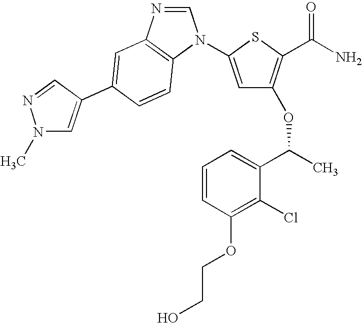

Methyl 3-{[(1R)-1-(2-chloro-3-hydroxyphenyl)ethyl]oxy}-5-[5-(1-methyl-1H-pyrazol-4-yl)-1H-benzimidazol-1-yl]-2-thiophenecarboxylate (Intermediate 8, 0.10 g, 0.20 mmol), 2-bromoethanol (0.029 g, 0.24 mmol) and K₂CO₃ (0.055 g, 0.40 mmol) were combined using the procedure analogous to Example 5, except to purify, the mixture was cooled, evaporated under reduced pressure, loaded onto a pre-packed solid loading cartridge using a minimal amount of DCM and subjected to a gradient elution using DCM (100%) to DCM:MeOH (80:20:) using a RediSep silica gel cartridge (12 g; ISCO). The appropriate fractions were combined and concentrated under reduced pressure to give 0.084 g (78%) of the title compound as a white solid. ¹H NMR (300 MHz, DMSO-d₆) δ 8.58 (s, 1H), 8.23 (s, 1H), 7.99 (s, 1H), 7.97 (s, 1H), 7.85 (s, 1H), 7.63 (dd, J=8.4, 1.6 Hz, 1H), 7.50 (d, J=9.1 Hz, 1H), 7.40 (t, J=8.0 Hz, 1H), 7.25 (d, J=8.1 Hz, 1H), 7.18-7.16 (m, 2H), 6.08-6.02 (m, 1H), 4.95 (t, J=5.4 Hz, 3H), 4.14-4.11 (m, 2H), 3.90 (s, 3H), 3.79 (q, J=5.3 Hz, 2H), 1.75 (d, J=5.3 Hz, 3H); MS (ESI) m/z 538.20 (M+H)⁺.

### Example 7

### 3-[((1R)-1-{3-[(2-aminoethyl)oxy]-2-chlorophenyl}ethyl)oxy]-5-[5-(1-methyl-1H-pyrazol-4-yl)-1H-benzimidazol-1-yl]-2-thiophenecarboxamide hydrochloride

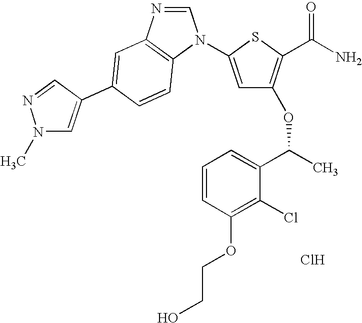

Methyl 3-{[(1R)-1-(2-chloro-3-hydroxyphenyl)ethyl]oxy}-5-[5-(1-methyl-1H-pyrazol-4-yl)-1H-benzimidazol-1-yl]-2-thiophenecarboxylate (Intermediate 8, 0.10 g, 0.20 mmol), 2-(Boc-amino)ethyl bromide (0.054 g, 0.24 mmol) and K₂CO₃ (0.055 g, 0.40 mmol) were combined using the procedure analogous to Example 5, except to purify, the mixture was concentrated to dryness and triturated using ether/MeOH (8:1) to give 0.093 g of a yellow solid. The yellow solid was stirred in MeOH with 1 mL of HCl in dioxane (4N) for 16 h after which time it was concentrated to give 0.11 g (100%) of yellow solid. ¹H NMR (400 MHz, DMSO-d₆) δ 8.67 (s, 1H), 8.20 (s, 1H), 8.10 (s, 2H), 7.93 (d, J=6.7 Hz, 2H), 7.83 (s, 1H), 7.59 (s, 1H), 7.37 (t, J=7.8 Hz, 1H), 7.28 (d, J=7.7 Hz, 1H), 7.16-7.13 (m, 2H), 6.02-5.99 (m, 1H), 4.23 (t, J=5.3 Hz, 2H), 3.84 (s, 3H), 3.69-3.62 (m, 1H), 3.49-3.41 (m, 1H), 3.23-3.17 (m, 2H), 1.69 (d, J=4.9 Hz, 3H). MS (ESI) m/z 537.16 (M+H)⁺.

### Example 8

### 3-{[(1R)-1-(2-chloro-3-{[2-(dimethylamino)ethyl]oxy}phenyl)ethyl]oxy}-5-[5-(1-methyl-1H-pyrazol-4-yl)-1H-benzimidazol-1-yl]-2-thiophenecarboxamide

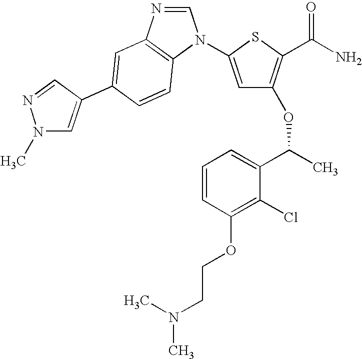

Methyl 3-{[(1R)-1-(2-chloro-3-hydroxyphenyl)ethyl]oxy}-5-[5-(1-methyl-1H-pyrazol-4-yl)-1H-benzimidazol-1-yl]-2-thiophenecarboxylate (Intermediate 8, 0.10 g, 0.20 mmol), 2-(dimethylamino) ethyl chloride hydrochloride (0.058 g, 0.40 mmol) and K₂CO₃ (0.14 g, 1.0 mmol) were combined using the procedure analogous to Example 6 to give 0.076 g (66%) of the title compound as a yellow solid. ¹H NMR (400 MHz, DMSO-d₆) δ 8.52 (s, 1H), 8.17 (s, 1H), 7.93 (s, 1H), 7.90 (s, 1H), 7.80 (s, 1H), 7.53 (d, J=8.1 Hz, 1H), 7.43 (d, J=8.5 Hz, 1H), 7.34 (t, J=8.1 Hz, 1H), 7.19 (d, J=7.4 Hz, 1H), 7.12-7.08 (m, 3H), 6.00-5.95 (m, 1H), 4.12 (t, J=5.7 Hz, 2H), 3.84 (s, 3H), 2.66-2.63 (m, 2H), 2.19 (s, 6H), 1.69 (d, J=5.1 Hz, 3H). MS (ESI) m/z 565.27 (M+H)⁺.

### Example 9

### 3-{[(1R)-1-(2-chloro-3-{[3-(dimethylamino)propyl]oxy}phenyl)ethyl]oxy}-5-[5-(1-methyl-1H-pyrazol-4-yl)-1H-benzimidazol-1-yl]-2-thiophenecarboxamide

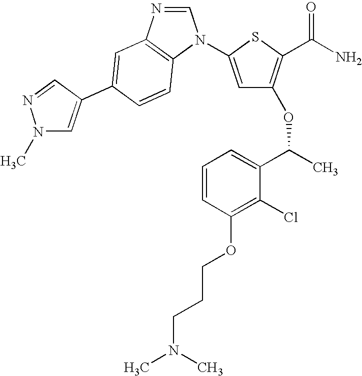

Methyl 3-{[(1R)-1-(2-chloro-3-hydroxyphenyl)ethyl]oxy}-5-[5-(1-methyl-1H-pyrazol-4-yl)-1H-benzimidazol-1-yl]-2-thiophenecarboxylate (Intermediate 8, 0.10 g, 0.20 mmol), dimethylamino-1-propanol (0.023 g, 0.22 mmol), triphenylphosphine (0.079 g, 0.30 mmol) and di-tert-butyl azodicarboxylate (0.056 g, 0.24 mmol) were combined in DCM (5 mL) using the procedure analogous to Intermediate 8, Step A to give 0.20 g of solid which was then combined with 2 mL of ammonia in MeOH (7N) in a sealed vessel and heated while stirring at 95° C. behind a blast shield for 16 h. The mixture was cooled, evaporated under reduced pressure, loaded onto a pre-packed solid loading cartridge using a minimal amount of DCM and subjected to a gradient elution using DCM (100%) to DCM:MeOH (80:20:) using a RediSep silica gel cartridge (12 g; ISCO). The appropriate fractions are combined and concentrated under reduced pressure to give 0.076 g (66%) of the title compound as an off-white solid. ¹H NMR (400 MHz, DMSO-d₆) δ 8.53 (s, 1H), 8.18 (s, 1H), 7.94 (s, 1H), 7.91 (s, 1H), 7.82 (s, 1H), 7.55 (dd, J=8.4, 1.2 Hz, 1H), 7.49 (d, J=8.3 Hz, 1H), 7.35 (t, J=8.1 Hz, 1H), 7.20 (d, J=7.4 Hz, 1H), 7.11-7.08 (m, 3H), 6.02-5.97 (m, 1H), 4.07 (t, J=5.9 Hz, 2H), 3.86 (s, 3H), 2.40-2.36 (m, 2H), 2.10 (s, 6H), 1.85 (m, 2H), 1.70 (d, J=6.1 Hz, 3H). MS (ESI) m/z 579.31 (M+H)⁺.

### Biological Examples

### I. Assay for Inhibition of PLK1

A. Preparation of 6×N-terminal His-tagged PLK kinase domain

6× N-terminal His-tagged PLK kinase domain (amino acids 21-346 preceded by MKKGHHHHHHD) SEQ ID: No. 1. was prepared from baculovirus infected T. ni cells under polyhedrin promoter control. All procedures were performed at 4° C. Cells were lysed in 50 mM HEPES, 200 mM NaCl, 50 mM imidazole, 5% glycerol; pH 7.5. The homogenate was centrifuged at 14K rpm in a SLA-1500 rotor for 1 hr and the supernatant filtered through a 1.2 micron filter. The supernatant was loaded onto a Nickel chelating Sepharose (Amersham Pharmacia) column and washed with lysis buffer. Protein was eluted using 20%, 30% and 100% buffer B steps where buffer B was 50 mM HEPES, 200 mM NaCl, 300 mM imidazole, 5% glycerol; pH 7.5. Fractions containing PLK were determined by SDS-PAGE. Fractions containing PLK were diluted five-fold with 50 mM HEPES, 1 mM DTT, 5% glycerol; pH 7.5, then loaded on an SP Sepharose (Amersham Pharmacia) column. After washing the column with 50 mM HEPES, 1 mM DTT, 5% glycerol; pH 7.5, PLK was step eluted with 50 mM HEPES, 1 mM DTT, 500 mM NaCl; 5% glycerol; pH 7.5. PLK was concentrated using a 10 kDa molecular weight cutoff membrane and then loaded onto a Superdex 200 gel filtration (Amersham Pharmacia) column equilibrated in 25 mM HEPES, 1 mM DTT, 500 mM NaCl, 5% glycerol; pH 7.5. Fractions containing PLK were determined by SDS-PAGE. PLK was pooled, aliquoted and stored at −80° C. Samples were quality controlled using mass spectrometry, N-terminal sequencing and amino acid analysis.

B. Enzyme activity+/−inhibitors was determined as follows:

All measurements were obtained under conditions where signal production increased linearly with time and enzyme. Test compounds were added to white 384-well assay plates (0.1 μL for 10 μL and some 20 μL assays, 1 μL for some 20 μL assays) at variable known concentrations in 100% DMSO. DMSO (I-5% final, as appropriate) and EDTA (65 mM in reaction) were used as controls. Reaction Mix was prepared as follows at 22° C.:
25 mM HEPES, pH 7.2
15 mM MgCl₂
1 μM ATP
0.05 μCi/well ³³P-γ ATP (10 Ci/mMol)
1 μM substrate peptide (Biotin-Ahx-SFNDTLDFD) SEQ ID: No. 2.
0.15 mg/mL BSA
1 mM DTT
2 nM PLK1 kinase domain (added last)

Reaction Mix (10 or 20 μL) was quickly added to each well immediately following addition of enzyme via automated liquid handlers and incubated 1-1.5 h at 22° C. The 20 μL enzymatic reactions were stopped with 50 μL of stop mix (50 mM EDTA, 4.0 mg/mL Streptavidin SPA beads in Standard Dulbecco's PBS (without Mg²⁺ and Ca²⁺), 50 μM ATP) per well. The 10 μL reactions were stopped with 10 μL of stop mix (50 mM EDTA, 3.0 mg/mL Streptavidin-coupled SPA Imaging Beads (“LeadSeeker”) in Standard Dulbecco's PBS (without Mg²⁺ and Ca²⁺), 50 μM ATP) per well. Plates were sealed with clear plastic seals, spun at 500×g for 1 min or settled overnight, and counted in Packard TopCount for 30 seconds/well (regular SPA) or imaged using a Viewlux imager (LeadSeeker SPA). Signal above background (EDTA controls) was converted to percent inhibition relative to that obtained in control (DMSO-only) wells.

C. Results

The data obtained is reported in Table 1 below. In Table 1, +=pIC₅₀<6; ++=pIC₅₀ 6-7; +++=pIC₅₀>7.

### II. Inhibition of Cell Proliferation by PLK1 Inhibitors

Exponentially growing cell lines of different tumor origins, cultured in appropriate media containing 10% fetal bovine serum at 37° C. in a 5% CO₂ incubator were plated at low density (less than 2000 cells/well) in 96-well plates. Twenty four hours post-plating, cells were treated with different concentrations of test compounds ranging from 10 uM to 0.04 nM. Several wells were left untreated as a control. Seventy two hours post-treatment, cell numbers were determined using different techniques; 100 μl per well of methylene blue (Sigma M9140) (0.5% in 50:50 Ethanol:water), or 50-100 ul per well of CellTiter-Glo (Promega #G7573). For methylene blue staining, stain was incubated at room temperature for 30 minutes before plates were rinsed and dye solubilized in 1% N-lauroyl sarcosine, sodium salt, (Sigma L5125, in PBS). Plates were read on a microplate reader, measuring the OD at 620 nm. For CellTiter-Glo, plates were incubated at room temperature for 15 minutes and the chemiluminescent signal was read on the Victor V or Envison 2100 reader.

Percent inhibition of cell growth was expressed as percent proliferation relative to 100% proliferation (control). Concentration of test compound that inhibited 50% of cell growth (IC₅₀) was determined by 4 parameter fit of data using XLfit, (value of no cell control was substracted from all samples for background). The data are shown in Table 1 and Table 2 below and represent a compilation of several different experiments each performed using the general parameters outlined above, although minor variations may have been employed in some instances. In Table 1 and Table 2, +=IC₅₀>1+M; ++=IC₅₀ 0.5-1 μM: +++=IC₅₀<0.5 μM.

TABLE 1

| Example | PLK1 pIC₅₀ | HCT116 IC₅₀ |
|---|---|---|
| 1 | ++ | |
| 2 | | +++ |
| 3 | +++ | +++ |
| 4 | +++ | +++ |
| 5 | +++ | +++ |
| 6 | +++ | +++ |
| 7 | +++ | +++ |
| 8 | +++ | +++ |
| 9 | +++ | +++ |

What is claimed is:

1. A compound of formula (I): 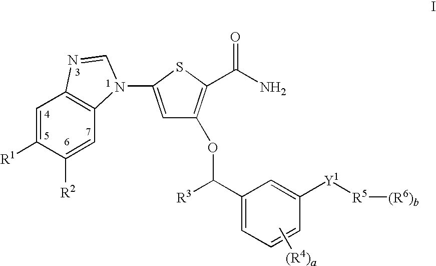
wherein:
R¹ and R² are the same or different and are each selected from H, halo, alkyl, haloalkyl, —OR⁷, —O-haloalkyl, —CN, —S(O)₂R⁷, —R⁵—S(O)₂R⁷, —NR⁷R⁸, and Het¹;
Het¹ is a 5-6 membered heteroaryl having 1 or 2 heteroatoms selected from N, O and S, optionally substituted 1 or 2 times with a substituent selected from alkyl and oxo;
R³ is H or alkyl;
a is 0, 1 or 2;
each R⁴ is the same or different and is halo;
Y¹ is —O—, —N(R⁷)—, —C(O)N(H)— or —N(H)C(O)—;
R⁵ is C₁₋₃alkylene;
b is 1 or 2;
each R⁶ is the same or different and is independently selected from —OR⁷ and —NR⁷R⁸; and
each R⁷ and each R⁸ are the same or different and are each independently selected from H, alkyl, alkenyl, alkynyl, cycloalkyl and cycloalkenyl;
or a pharmaceutically acceptable salt thereof.

2. The compound according to claim 1, wherein R¹ is selected from H, halo, —OR⁷, and Het¹.

3. The compound according to claim 1, wherein R² is selected from H, halo and —OR⁷.

4. The compound according to claim 1, wherein R³ is alkyl.

5. The compound according to claim 1, wherein a is 1 and R⁴ is Cl.

6. The compound according to claim 1, wherein Y¹ is —O—.

7. The compound according to claim 1, wherein a is R⁵ is ethylene or n-propylene.

8. The compound according to claim 1, wherein b is 1.

9. The compound according to claim 1, wherein b is 1 and R⁶ is selected from —OH, —Oalkyl, —NH₂, —N(H)alkyl and —N(alkyl)₂.

10. An enantiomerically enriched compound according to claim 1, having the stereochemistry depicted in formula (I-1): 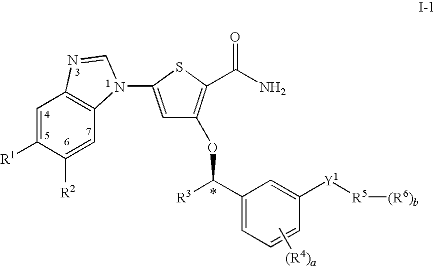
wherein * indicates the chiral carbon and all variables are as defined in claim 1.

11. A compound, according to claim 1 selected from
5-[5,6-Bis(methyloxy)-1H-benzimidazol-1-yl]-3-({(1R)-1-[2-chloro-5-({[2-(dimethylamino)ethyl]amino}carbonyl)phenyl]ethyl}oxy)-2-thiophenecarboxamide formate;
5-[5,6-Bis(methyloxy)-1H-benzimidazol-1-yl]-3-[((1R)-1-{2-chloro-3-[(2-hydroxyethyl)amino]phenyl}ethyl)oxy]-2-thiophenecarboxamide;
3-[((1R)-1-{3-[(2-aminoethyl)oxy]-2-chlorophenyl}ethyl)oxy]-5-[5,6-bis(methyloxy)-1H-benzimidazol-1-yl]-2-thiophenecarboxamide;
3-[((1R)-1-{3-[(2-Aminoethyl)oxy]-2-chlorophenyl}ethyl)oxy]-5-(1H-benzimidazol-1-yl)-2-thiophenecarboxamide;
3-[((1R)-1-{2-chloro-3-[(3-hydroxypropyl)oxy]phenyl}ethyl)oxy]-5-[5-(1-methyl-1H-pyrazol-4-yl)-1H-benzimidazol-1-yl]-2-thiophenecarboxamide;
3-[((1R)-1-{2-chloro-3-[(2-hydroxyethyl)oxy]phenyl}ethyl)oxy]-5-[5-(1-methyl-1H-pyrazol-4-yl)-1H-benzimidazol-1-yl]-2-thiophenecarboxamide;
3-[((1R)-1-{3-[(2-aminoethyl)oxy]-2-chlorophenyl}ethyl)oxy]-5-[5-(1-methyl-1H-pyrazol-4-yl)-1H-benzimidazol-1-yl]-2-thiophenecarboxamide hydrochloride;
3-{[(1R)-1-(2-chloro-3-{[2-(dimethylamino)ethyl]oxy}phenyl)ethyl]oxy}-5-[5-(1-methyl-1H-pyrazol-4-yl)-1H-benzimidazol-1-yl]-2-thiophenecarboxamide; and
3-{[(1R)-1-(2-chloro-3-{[3-(dimethylamino)propyl]oxy}phenyl)ethyl]oxy}-5-[5-(1-methyl-1H-pyrazol-4-yl)-1H-benzimidazol-1-yl]-2-thiophenecarboxamide,
and pharmaceutically acceptable salts thereof.

12. (canceled)

13. A pharmaceutical composition according to claim 1 comprising a compound of claim 1 and a pharmaceutically acceptable carrier, diluent or excipient.

14. A method for treating a susceptible neoplasm in a human in need thereof, said method comprising administering to the human a therapeutically effective amount of a compound according to claim 1.

15. The method according to claim 14, wherein said susceptible neoplasm is selected from the group consisting of breast cancer, colon cancer, small cell lung cancer, non-small cell lung cancer, prostate cancer, endometrial cancer, gastric cancer, melanoma, ovarian cancer, pancreatic cancer, squamous cell carcinoma, carcinoma of the head and neck, esophageal carcinoma, hepatocellular carcinoma, and hematologic malignancies.

16. A method for treating a condition characterized by inappropriate cellular proliferation in a mammal in need thereof, said method comprising administering to the mammal a therapeutically effective amount of a compound according to claim 1.

17. A process for preparing a compound according to claim 1 wherein Y¹ is —O—, said process comprising the steps of:
a) reacting the compound of formula (VII): 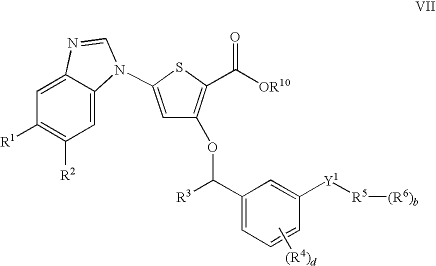
wherein:
R¹⁰ is selected from alkyl and suitable carboxylic acid protecting groups;
Y¹ is —O—;
with ammonia to prepare a compound of formula (I);
b) optionally separating the compound of formula (I) into enantiomers;
c) optionally converting the compound of formula (I) to a pharmaceutically acceptable salt thereof; and
d) optionally converting the compound of formula (I) or a pharmaceutically acceptable salt or solvate thereof to a different compound of formula (I) or a pharmaceutically acceptable salt thereof.

18. A process for preparing a compound according to claim 1 wherein Y¹ is —N(R⁷)— or —N(H)C(O)—, said process comprising the steps of:
a) reacting the compound of formula (XXXIII): 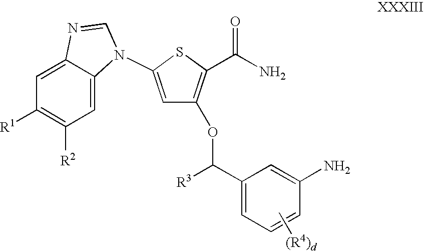
b) with a compound of formula (XXXIV) or (XXXV): 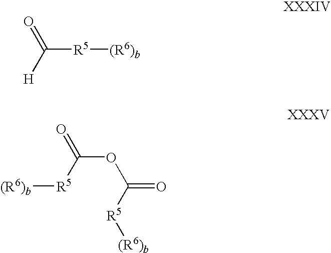
to prepare a compound of formula (I);
c) optionally separating the compound of formula (I) into enantiomers;
d) optionally converting the compound of formula (I) to a pharmaceutically acceptable salt or solvate thereof; and
e) optionally converting the compound of formula (I) or a pharmaceutically acceptable salt thereof to a different compound of formula (I) or a pharmaceutically acceptable salt thereof.

19. (canceled)

20. (canceled)

21. (canceled)

22. (canceled)

23. (canceled)

24. (canceled)

25. (canceled)

26. (canceled)
